(19) United States
(12) Patent Application Publication
(10) Pub. No.: US 2022/0169672 A1
(43) Pub. Date: Jun. 2, 2022

(54) COMPOUNDS FOR TARGETED THERAPIES OF CASTRATION RESISTANT PROSTATE CANCER

(71) Applicant: Purdue Research Foundation, West Lafayette, IN (US)

(72) Inventor: Gaurav Chopra, West Lafayette, IN (US)

(21) Appl. No.: 17/434,672

(22) PCT Filed: Feb. 28, 2020
(86) PCT No.: PCT/US2020/020332

§ 371 Date: Aug. 27, 2021

Related U.S. Application Data
(60) Provisional application No. 62/811,747, filed on Feb. 28, 2019.

(51) Int. Cl. C07J 13/00; A61P 35/00; C07J 41/00

(52) U.S. Cl. CPC C07J 13/005; A61P 35/00; C07J 41/0027; C07J 41/0094; C07J 41/0022

(57) ABSTRACT

The present invention generally relates to new compounds for therapeutic uses. In particular, this disclosure relates to novel tetracyclic compounds useful for treatment of cancer, especially castration resistant prostate cancer. Pharmaceutical composition matters and methods for treating a cancer patient by administering therapeutically effective amounts of such compound alone or together with other therapeutics are within the scope of this disclosure.

### CROSS REFERENCE TO RELATED APPLICATIONS

This present patent application relates to and claims the priority benefit of U.S. Provisional Application Ser. No. 62/811,747, filed Feb. 28 2019, the content of which is hereby incorporated by reference in its entirety into this instant disclosure.

### TECHNICAL FIELD

The present invention generally relates to new compounds for therapeutic uses. In particular, this disclosure relates to novel tetracyclic compounds useful for treatment of cancer, especially castration resistant prostate cancer. Also described herein are pharmaceutical compositions of such compounds and methods for treating a cancer patient by administering therapeutically effective amounts of such compound alone, together with other therapeutics, or in a pharmaceutical composition

### BACKGROUND

This section introduces aspects that may help facilitate a better understanding of the disclosure. Accordingly, these statements are to be read in this light and are not to be understood as admissions about what is or is not prior art.

Prostate cancer is the most common malignancy in aging males and the second leading cause of death by cancer in men in the United States. The majority of prostate cancer are dependent on androgens (such as testosterone) for their growth and progression and androgen deprivation therapy (ADT)—is the mainstay therapy for patients with advanced prostate cancer. However, despite an initial response, prostate cancer almost always eventually acquires resistance to androgen depletion and it is termed as castration-resistant prostate cancer (CRPC). There are unmet medical needs for more effective treatment of cancer, especially CRPC.

### BRIEF SUMMARY OF INVENTION

The present invention generally relates to new compounds for therapeutic uses. In particular, this disclosure relates to novel tetracyclic compounds useful for treatment of cancer, especially castration resistant prostate cancer.

Also described herein are pharmaceutical compositions of such compounds and methods for treating a cancer patient by administering therapeutically effective amounts of such compound alone, together with other therapeutics, or in a pharmaceutical composition.

In some illustrative embodiments, the present invention relates to a compound having the formula (I)

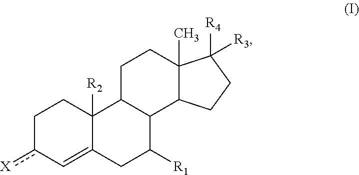

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (II)

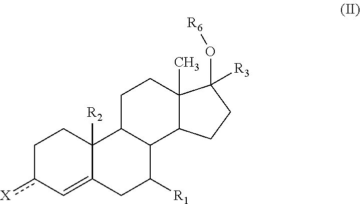

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (III)

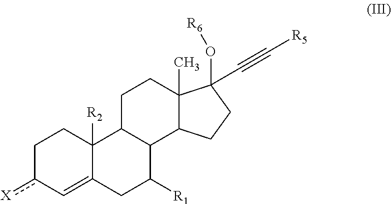

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₅ is hydrogen, an alkyl, alkenyl, alkynyl, alkylalkynyl, hydroxyalkyl, amino alkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (III) as disclosed herein, wherein [symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen or methyl;
R₅ is hydrogen, an alkyl, alkenyl, alkynyl, alkylalkynyl, hydroxyalkyl, amino alkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen or a C1-C6 alkyl.

In some illustrative embodiments, the present invention relates to a compound having the formula (III) as disclosed herein, wherein [symbol] represents a single or double bond, wherein [symbol] when [symbol] represents a single bond, X is a hydroxyl or alkyloxy; or when represents a double bond, X is O, S, NH, N—OH, N—NH₂, or NR₇, wherein R₇ is an C1-C6 alkyl;
R₁ and R₂ are independently hydrogen or methyl; and
R₅ and R₆ are hydrogen.

In some illustrative embodiments, the present invention relates to a compound having the formula (III), wherein said compound is a compound of compounds 1-6 of FIG. 2.

In some illustrative embodiments, the present invention relates to a compound having the formula (III), wherein said compound is a compound of compounds 15-22 of FIG. 2.

In some illustrative embodiments, the present invention relates to a compound having the formula (III), wherein said compound is a compound of compounds 23-40 of FIG. 2.

In some illustrative embodiments, the present invention relates to a compound having the formula (III) as disclosed herein, wherein [symbol] represents a double bond, and X is O, S, NH, N—OH N—NH₂, or NR₇, wherein R₇ is an C1-C6 alkyl;
R₁ and R₂ are independently hydrogen or methyl; and
R₅ and R₆ are hydrogen.

In some illustrative embodiments, the present invention relates to a compound having the formula (III) as disclosed herein, wherein [symbol] represents a single or double bond, wherein when [symbol] represents a single bond, X is a hydroxyl or alkyloxy; or when represents a double bond, X is O, S, NH, N—OH, N—NH₂, or NR₇, wherein R₇ is an C1-C6 alkyl;
R₁ and R₂ are independently hydrogen or methyl;
R₅ is

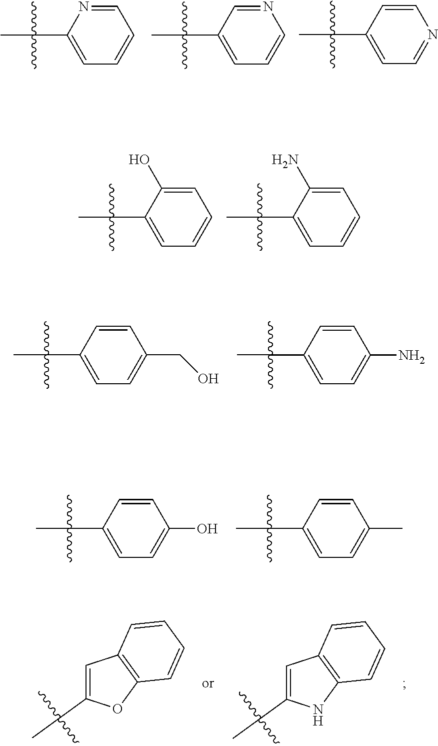

and
R₆ are hydrogen.

In some illustrative embodiments, the present invention relates to a compound having the formula (IV),

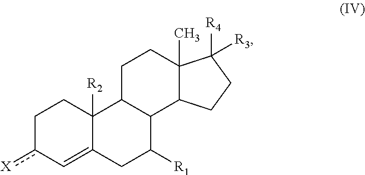

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (V),

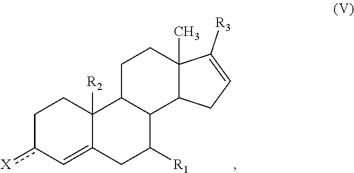

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (V) as disclosed herein, wherein the compound comprises a compound of compounds 7-14 of FIG. 2, or

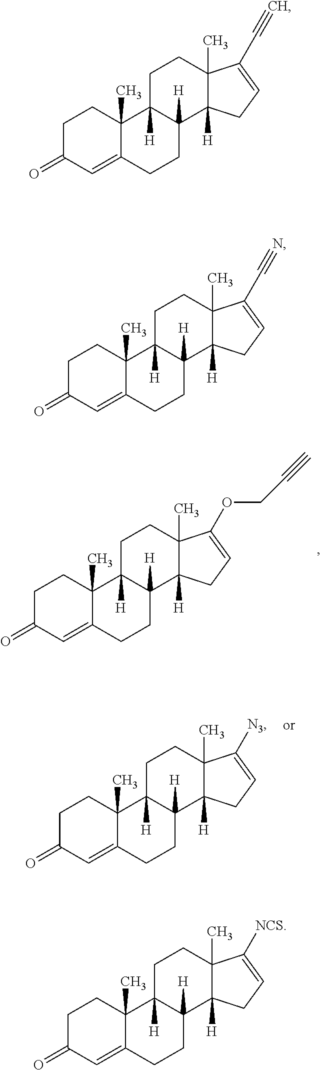

In some illustrative embodiments, the present invention relates to a pharmaceutical composition comprising one or more compounds as disclosed herein, or a pharmaceutically acceptable salt thereof, together with one or more diluents, excipients or carriers.

In some illustrative embodiments, the present invention relates to one or more compounds as disclosed herein, wherein the compound is for the treatment of a cancer.

In some illustrative embodiments, the present invention relates to one or more compounds as disclosed herein, wherein the compound is for the treatment of a cancer.

In some illustrative embodiments, the present invention relates to one or more compounds as disclosed herein, wherein the compound is for the treatment of castration resistant prostate cancer.

In some illustrative embodiments, the present invention relates to a method for treating a cancer patient, comprising the step of administering a therapeutically effective amount of one or more compounds as disclosed herein, and one or more carriers, diluents, or excipients, to a patient in need of relief from said cancer.

In some illustrative embodiments, the present invention relates to a method for treating a cancer patient, comprising the step of administering a therapeutically effective amount of one or more compounds as disclosed herein, and one or more carriers, diluents, or excipients, to a patient in need of relief from said castration resistant prostate cancer.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said cancer.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said castration resistant prostate cancer.

In some illustrative embodiments, the present invention relates to a pharmaceutical composition comprising one or more compounds as disclosed herein, or a pharmaceutically acceptable salt thereof, together with one or more diluents, excipients or carriers, for use as a medicament for cancer.

In some illustrative embodiments, the present invention relates to a method for treating a cancer patient, comprising the step of administering a therapeutically effective amount of one or more compounds, together with one or more carriers, diluents, or excipients, to a patient in need of relief from said cancer, the compound having the formula:

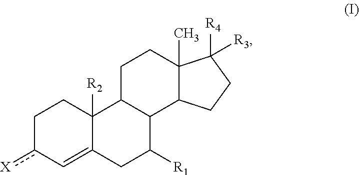

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said cancer is castration resistant prostate cancer.

In some illustrative embodiments, the present invention relates to a drug conjugate, wherein the drug conjugate comprises one or more compounds disclosed herein, wherein the conjugate confers cell-type or tissue type targeting or the conjugate targets another pathway that synergizes the action of compounds disclosed herein.

In some other illustrative embodiments, the present invention relates to a pharmaceutical composition comprising nanoparticles of one or more compounds disclosed herein, together with one or more diluents, excipients or carriers.

These and other features, aspects and advantages of the present invention will become better understood with reference to the following drawings, descriptions and claims.

### BRIEF DESCRIPTION OF THE DRAWINGS

The above and other objects, features, and advantages of the present invention will become more apparent when taken in conjunction with the following description and drawings, wherein:

FIGS. 1A-1F show parent lead compounds and their anti-cancer activity. FIG. 1A shows Cell viability IC₅₀ plots for the parent leads tibolone (TIB), norethisterone (NOR) and levonorgestrel (LEV) in LNCaP and C4-2 cells. FIG. 1B shows IC₅₀ graphs of the parent leads in normal human prostate epithelial RWPE-1 cell line. FIG. 1C shows Effect of the initial leads on the degradation of AR expression in Western blot in LNCaP and C4-2 cells. FIG. 1D shows AR expression in both LNCaP and C4-2 cells quantified from the western blots. FIG. 1E shows Immunofluorescent staining of LNCaP and C4-2 cells for AR expression after 24 h treatment with 1 μM of the indicated compounds. FIG. 1F shows Nuclear AR expression in both LNCaP and C4-2 cells quantified from the images of FIG. 1E. Tibolone, norethisterone and levonorgestrel were identified as active and non-toxic parent leads for CRPC.

FIG. 2A shows the structure of the parent leads Tibolone (TIB), Norethisterone (NOR) and Levonorgestrel (LEV) and general parent lead scaffold; FIG. 2B shows structures of the new small molecules (1-40).

FIGS. 3A-3F demonstrate the Anti-cancer activity of the synthetic molecules. (FIG. 3A) IC50 plots for the synthetic molecules 1-40, and abiraterone (ABI) in CRPC C4-2 cancer cell line. (FIG. 3B) IC50 plots for the active compounds and ABI in RWPE-1 normal cell line. (FIG. 3C) Western blot analysis for AR and β-actin (loading control) in C4-2 cells treated with vehicle and 1 μM concentration of the active compounds for 24 h. (FIG. 3D) and (FIG. 3E) are respective migration speed and wound closure rate in both LNCaP and C4-2 cells in presence of the active and non-toxic compounds. FIG. 3F shows metabolic stability of the active leads after 60 minutes of co-incubation with human and mouse liver microsomes respectively. Warfarin (WAR) is negative and verapamil (VER) is positive controls in mouse liver microsome assays.

FIGS. 4A-4B: Immunofluorescent staining of C4-2 cells for known target AR (FIG. 4A) and identified proteome targets RORG, SHBG and CYP17A1 expression after 24 h treatment with 1 μM of the indicated compounds (FIG. 4B.

FIGS. 5A-5B: Immunofluorescent staining of C4-2 cells for known targets AR and CYP17A1 (FIG. 5A) and identified proteome targets RORG and PR expression after 24 h treatment with 1 μM of the indicated compounds (FIG. 5B).

FIGS. 6A-6B: Immunofluorescent staining of C4-2 cells for known targets AR (FIG. 6A) and identified proteome targets RORG and PR expression after 24 h treatment with 1 μM of the indicated compounds (FIG. 6B).

FIGS. 7A-7C: LuCaP35 CRPC studies for the potent and non-toxic lead compound 1. (FIG. 7A) Schematic representation of the LUCaP35 CRPC mice model study. (FIG. 7B) Average tumor size plot for various treatments on LuCaP35 CRPC mice. (FIG. 7C) Average body weight change rate for all type of treatment mice. Synthetic lead 1 was more potent than the known drug abiraterone in LuCaP35 CRPC model.

### DETAILED DESCRIPTION

For the purposes of promoting an understanding of the principles of the present disclosure, references will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

A “halogen” designates F, CI, Br or I. A “halogen-substitution” or “halo” substitution designates replacement of one or more hydrogen atoms with F, CI, Br or I.

As used herein, the term “alkyl” refers to a saturated monovalent chain of carbon atoms, which may be optionally branched. It is understood that in embodiments that include alkyl, illustrative variations of those embodiments include lower alkyl, such as C₁-C₆ alkyl, methyl, ethyl, propyl, 3-methylpentyl, and the like.

As used herein, the term “alkenyl” refers to an unsaturated monovalent chain of carbon atoms including at least one double bond, which may be optionally branched. It is understood that in embodiments that include alkenyl, illustrative variations of those embodiments include lower alkenyl, such as C₂-C₆, C₂-C₄ alkenyl, and the like.

As used herein, the term “alkynyl” refers to an unsaturated monovalent chain of carbon atoms including at least one triple bond, which may be optionally branched. It is understood that in embodiments that include alkynyl, illustrative variations of those embodiments include lower alkynyl, such as C2-C₆, C₂-C₄ alkynyl, and the like.

As used herein, the term “cycloalkyl” refers to a monovalent chain of carbon atoms, a portion of which forms a ring. It is understood that in embodiments that include cycloalkyl, illustrative variations of those embodiments include lower cycloalkyl, such as C₃-C₈ cycloalkyl, cyclopropyl, cyclohexyl, 3-ethylcyclopentyl, and the like.

As used herein, the term “cycloalkenyl” refers to an unsaturated monovalent chain of carbon atoms, a portion of which forms a ring. It is understood that in embodiments that include cycloalkenyl, illustrative variations of those embodiments include lower cycloalkenyl, such as C₃-C₈, C₃-C₆ cycloalkenyl.

As used herein, the term “alkylene” refers to a saturated bivalent chain of carbon atoms, which may be optionally branched. It is understood that in embodiments that include alkylene, illustrative variations of those embodiments include lower alkylene, such as C2-C4, alkylene, methylene, ethylene, propylene, 3-methylpentylene, and the like.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialkylamino, acylamino, thio, and the like, and combinations thereof.

As used herein, the term “heterocyclic” or “heterocycle” refers to a monovalent chain of carbon and heteroatoms, wherein the heteroatoms are selected from nitrogen, oxygen, and sulfur, and a portion of which, at least one heteroatom, forms a ring. The term “heterocycle” may include both “aromatic heterocycles” and “non-aromatic heterocycles.” Heterocycles include 4-7 membered monocyclic and 8-12 membered bicyclic rings, such as imidazolyl, thiazolyl, oxazolyl, oxazinyl, thiazinyl, dithianyl, dioxanyl, isoxazolyl, isothiazolyl, triazolyl, furanyl, tetrahydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, piperidinyl, piperazinyl, pyrimidinyl, morpholinyl, tetrahydrothiophenyl, thiophenyl, azetidinyl, oxetanyl, thiiranyl, oxiranyl, aziridinyl, and the like. “Heterocycles” may be optionally substituted at any one or more positions capable of bearing a hydrogen atom.

As used herein, the term “aryl” includes monocyclic and polycyclic aromatic carbocyclic groups, each of which may be optionally substituted. The term “optionally substituted aryl” refers to an aromatic mono or polycyclic ring of carbon atoms, such as phenyl, naphthyl, and the like, which may be optionally substituted with one or more independently selected substituents, such as halo, hydroxyl, amino, alkyl, or alkoxy, alkylsulfony, cyano, nitro, and the like.

The term “heteroaryl” or “aromatic heterocycle” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” may also include ring systems having one or two rings wherein at least one of the rings is heteroaromatic, e.g., the other cyclic rings can be cycloalkyl, cycloalkenyl, cycloalkynyl, aromatic carbocycle, heteroaryl, and/or heterocycle. Heteroaryl groups include, without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

In some embodiments, “heterocycloalkyl” refers to a non-aromatic heterocycle where one or more of the ring-forming atoms are a heteroatom such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion.

The term “optionally substituted,” or “optional substituents,” as used herein, means that the groups in question are either unsubstituted or substituted with one or more of the substituents specified. When the groups in question are substituted with more than one substituent, the substituents may be the same or different. Furthermore, when using the terms “independently,” “independently are,” and “independently selected from” mean that the groups in question may be the same or different. Certain of the herein defined terms may occur more than once in the structure, and upon such occurrence each term shall be defined independently of the other.

The term “patient” includes human and non-human animals such as companion animals (dogs and cats and the like) and livestock animals. Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 carbo n atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 carbon atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil, soybean oil or the like.

Suitable solvents, gelatinizing agents and solubilizers are water or water miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of sodium lauryl sulphate, fatty alcohol ether sulphates, di-sodium-N-lauryl-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, a compound of the invention may be administered as a combination therapy with further active agents, e.g. therapeutically active compounds useful in the treatment of cancer, for example, prostate cancer, ovarian cancer, lung cancer, or breast cancer. For a combination therapy, the active ingredients may be formulated as compositions containing several active ingredients in a single dose form and/or as kits containing individual active ingredients in separate dose forms. The active ingredients used in combination therapy may be co-administered or administered separate

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, N.Y., pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about 5 mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of the compound with other conventional and non-conventional therapeutic treatments, including other anti-tumor agents, radiation therapy, and the like.

The present invention generally relates to new compounds for therapeutic uses. In particular, this disclosure relates to novel tetracyclic compounds useful for treatment of cancer, especially castration resistant prostate cancer.

Also described herein are pharmaceutical compositions of such compounds and methods for treating a cancer patient by administering therapeutically effective amounts of such compound alone, together with other therapeutics, or in a pharmaceutical composition.

In some illustrative embodiments, the present invention relates to a compound having the formula (I)

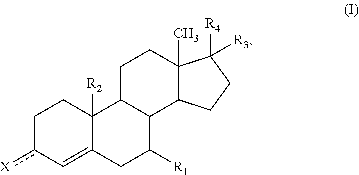

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (II)

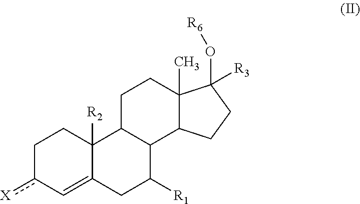

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (III)

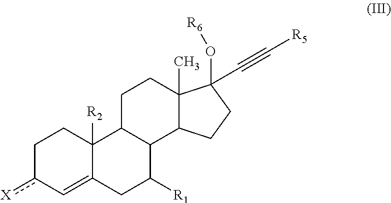

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₅ is hydrogen, an alkyl, alkenyl, alkynyl, alkylalkynyl, hydroxyalkyl, amino alkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (III) as disclosed herein, wherein [symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen or methyl;
R₅ is hydrogen, an alkyl, alkenyl, alkynyl, alkylalkynyl, hydroxyalkyl, amino alkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen or a C1-C6 alkyl.

In some illustrative embodiments, the present invention relates to a compound having the formula (III) as disclosed herein, wherein [symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen or methyl; and
R₅ and R₆ are hydrogen.

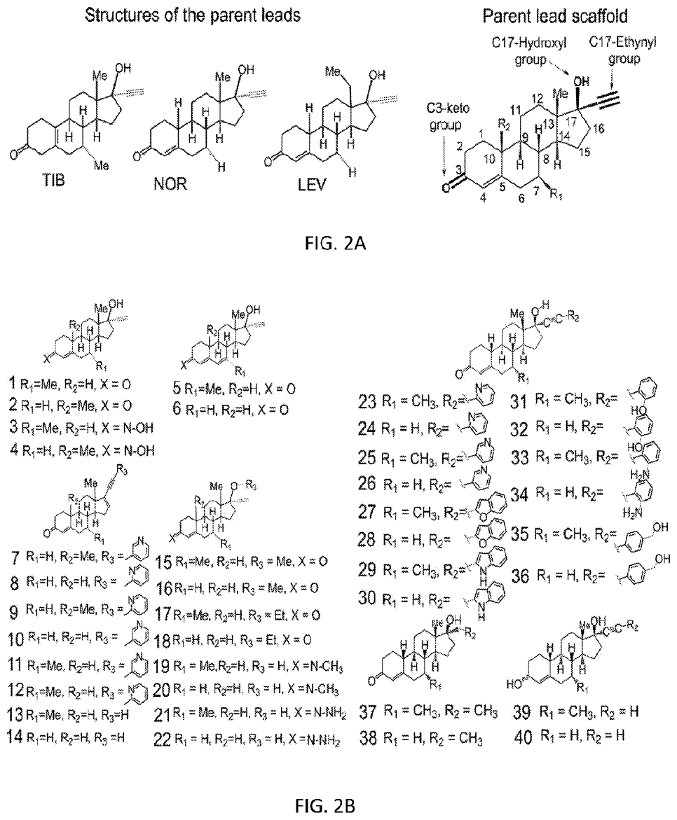

In some illustrative embodiments, the present invention relates to a compound having the formula (III), wherein said compound is a compound of compounds 1-6 of FIG. 2.

In some illustrative embodiments, the present invention relates to a compound having the formula (III), wherein said compound is a compound of compounds 15-22 of FIG. 2.

In some illustrative embodiments, the present invention relates to a compound having the formula (III), wherein said compound is a compound of compounds 23-40 of FIG. 2.

In some illustrative embodiments, the present invention relates to a compound having the formula (III) as disclosed herein, wherein [symbol] represents a double bond, and X is O, S, NH, N—OH, N—NH₂, or NR₇, wherein R₇ is an C1-C6 alkyl;
R₁ and R₂ are independently hydrogen or methyl; and
R₅ and R₆ are hydrogen.

In some illustrative embodiments, the present invention relates to a compound having the formula (III) as disclosed herein, wherein [symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen or methyl;
R₅ is

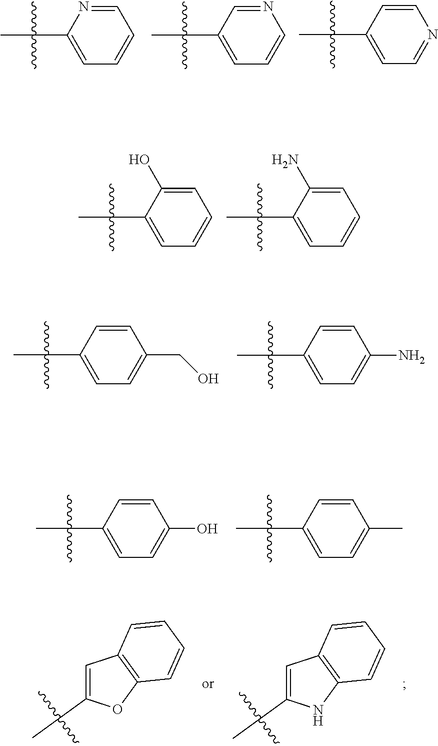

and
R₆ are hydrogen.

In some illustrative embodiments, the present invention relates to a compound having the formula (IV),

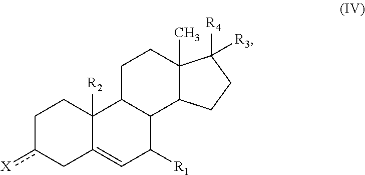

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (V),

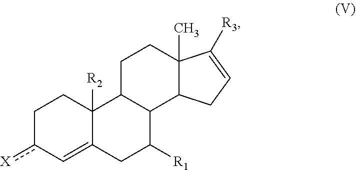

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having the formula (V) as disclosed herein, wherein the compound comprises a compound of compounds 7-14 of FIG. 2, or

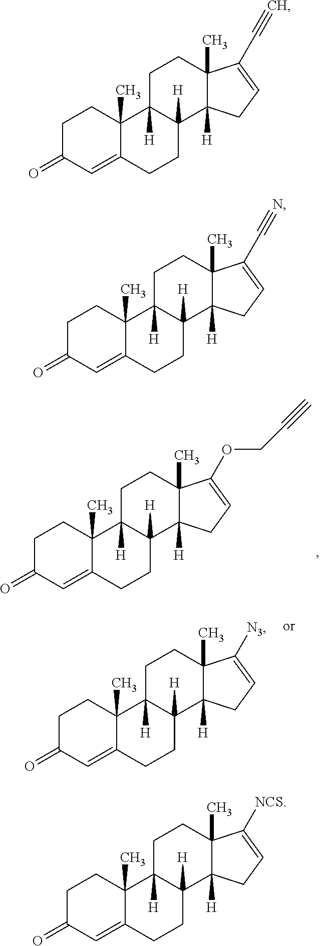

In some illustrative embodiments, the present invention relates to a compound having the formula (I) as disclosed herein, the compounds are

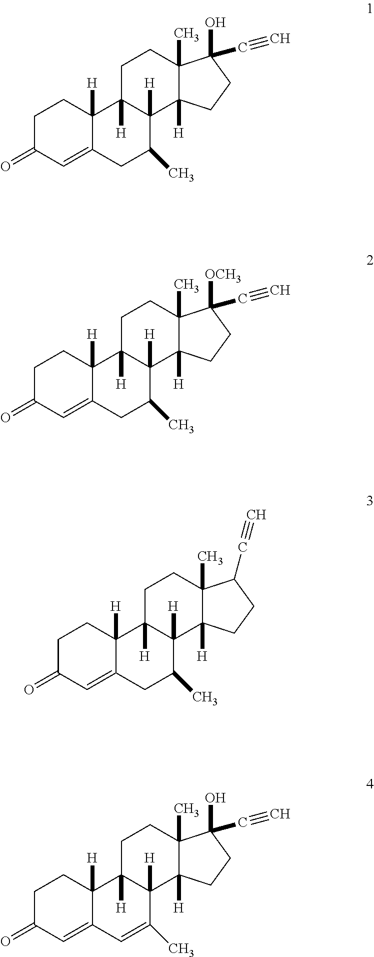

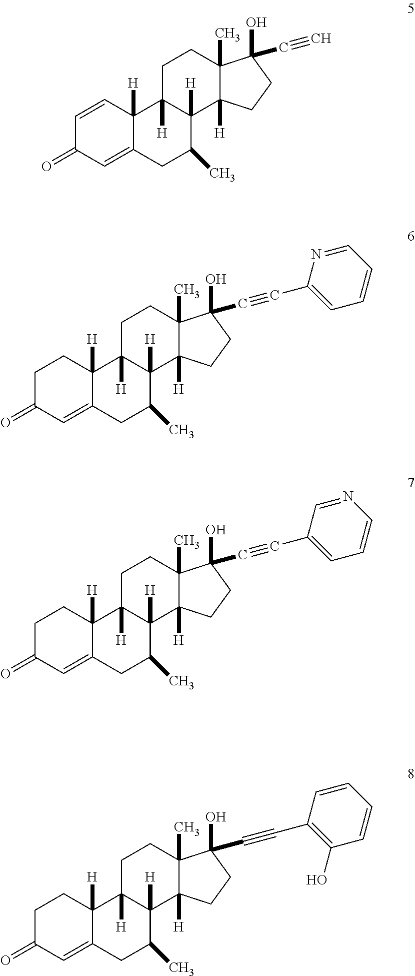

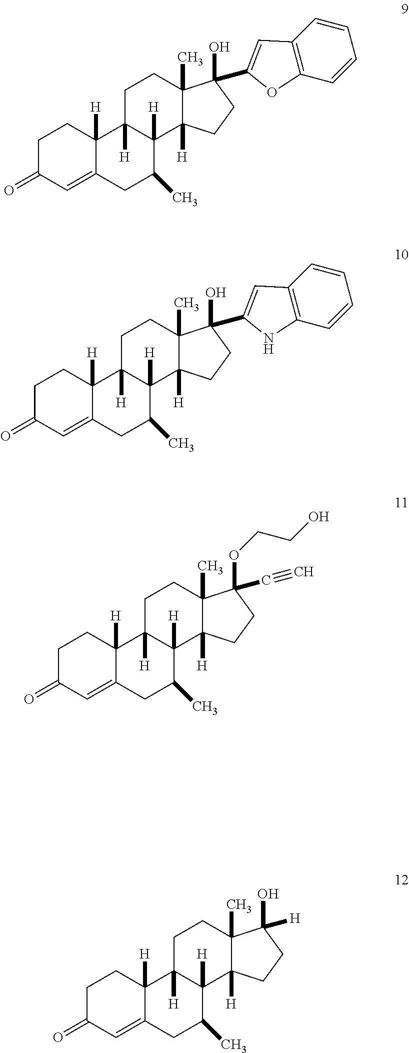

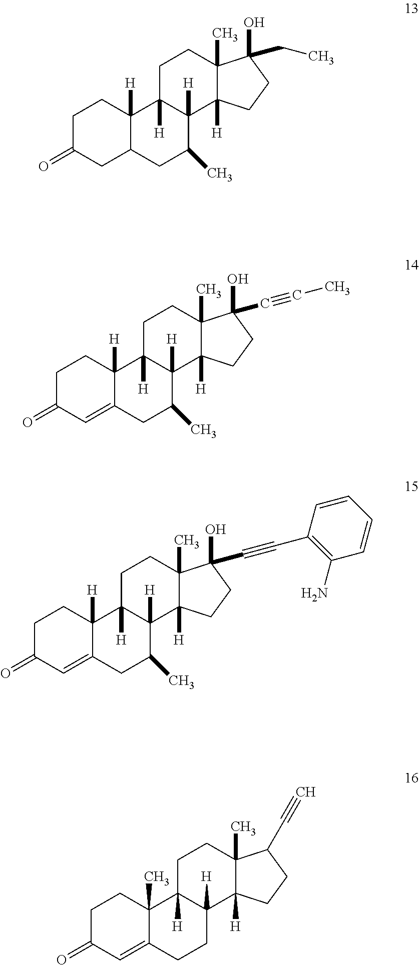

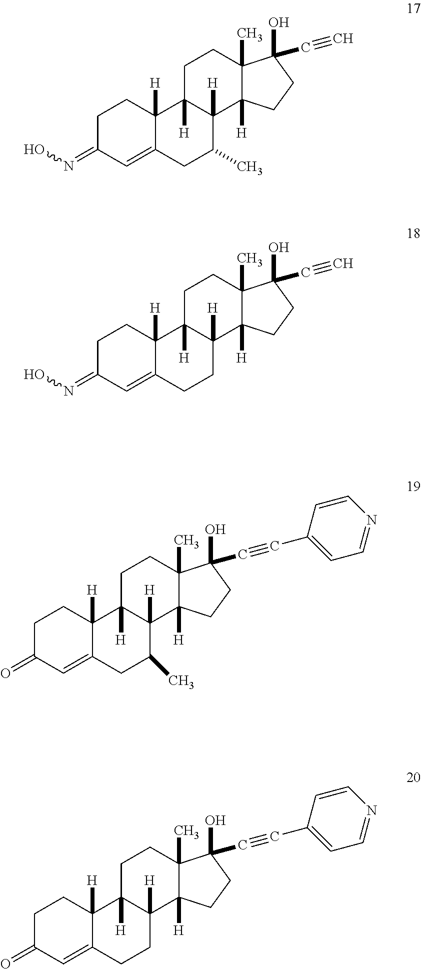

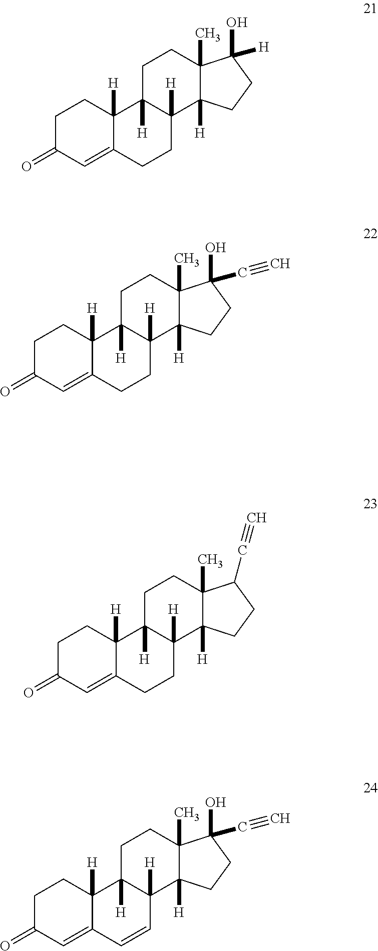

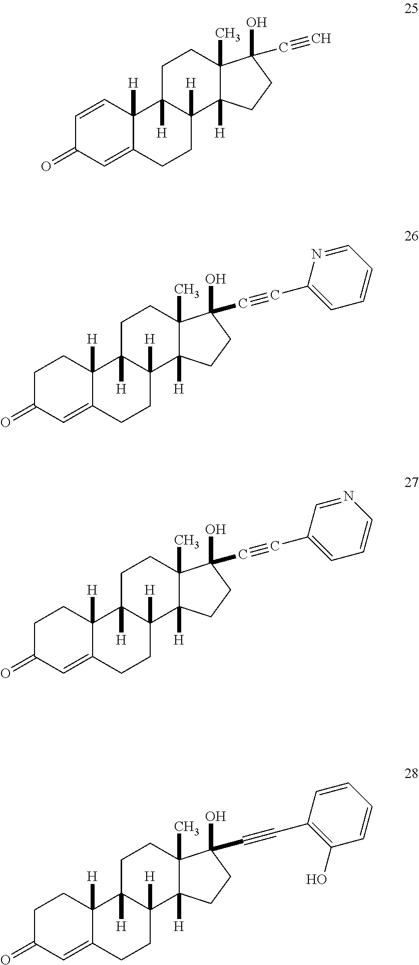

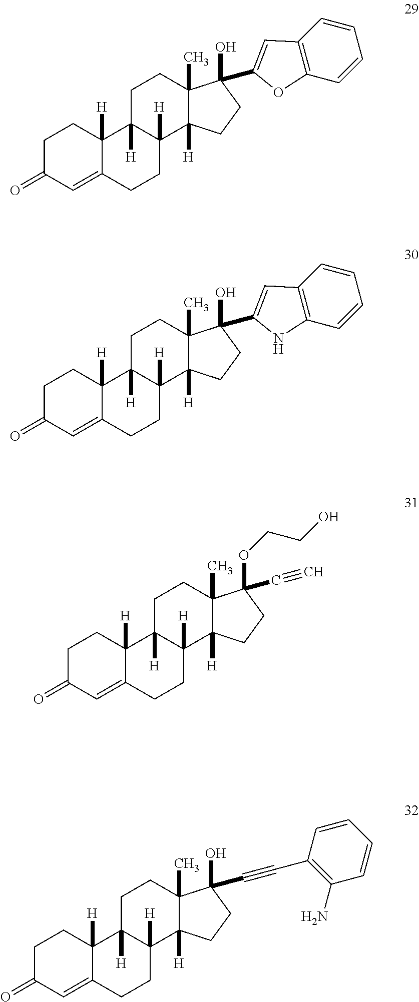

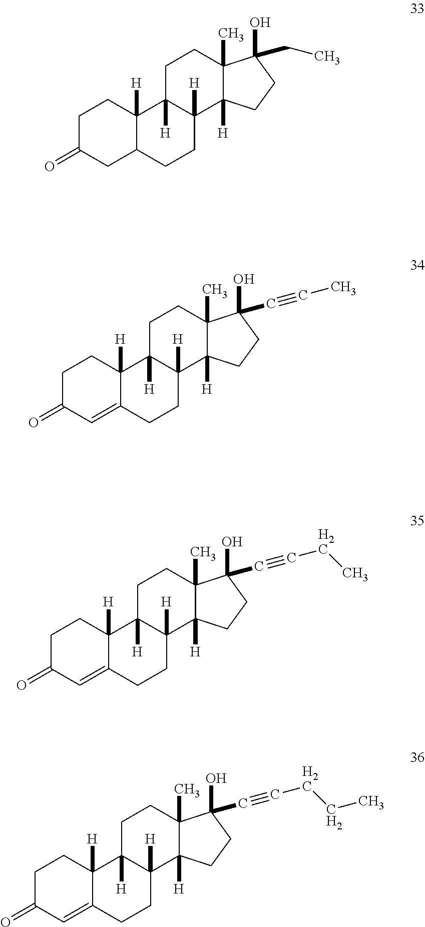

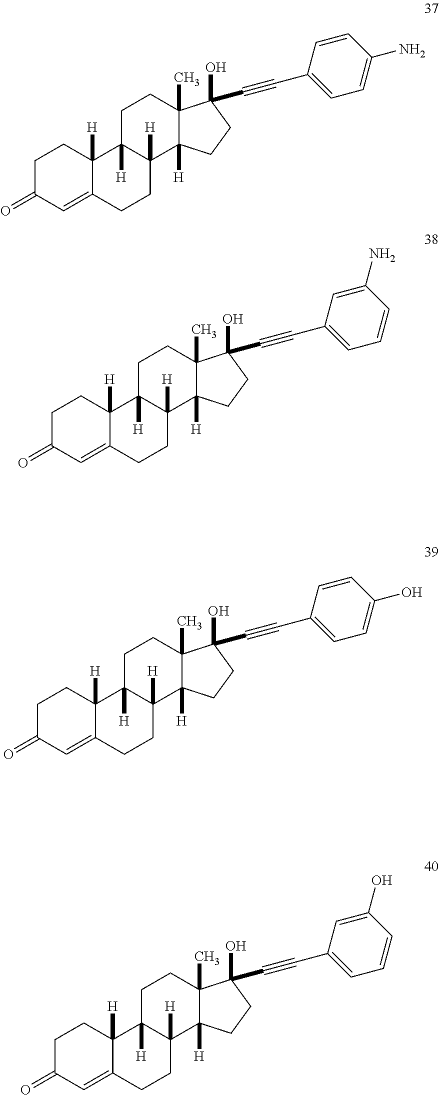

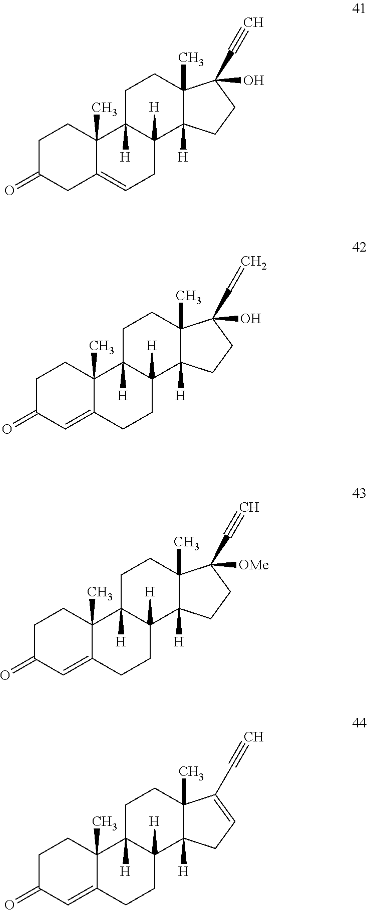

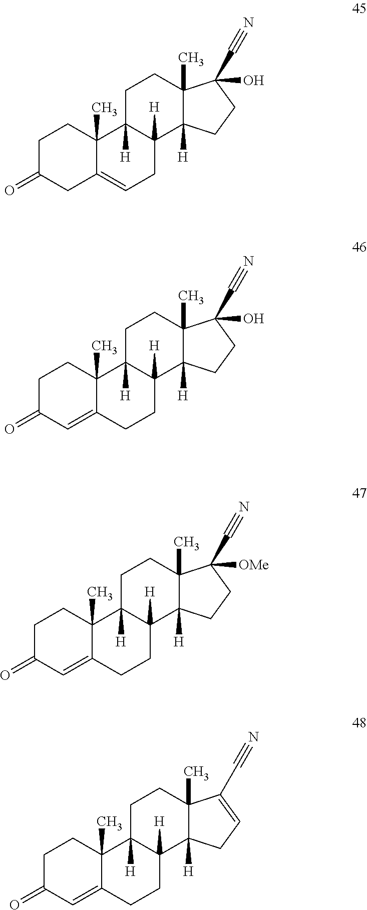

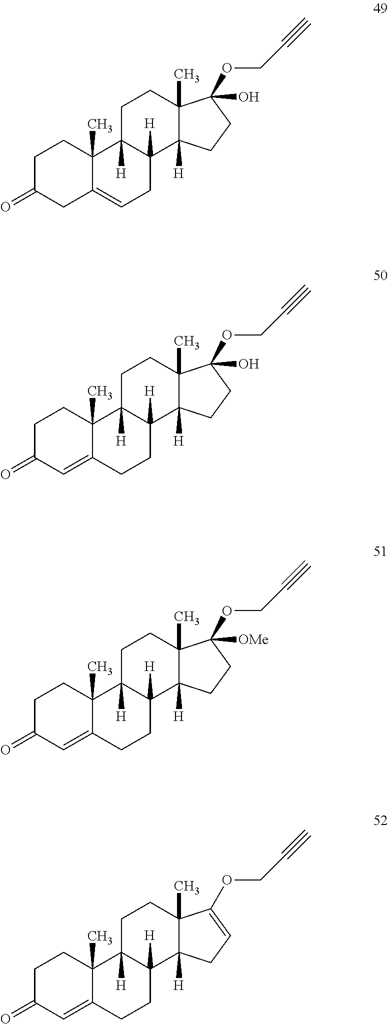

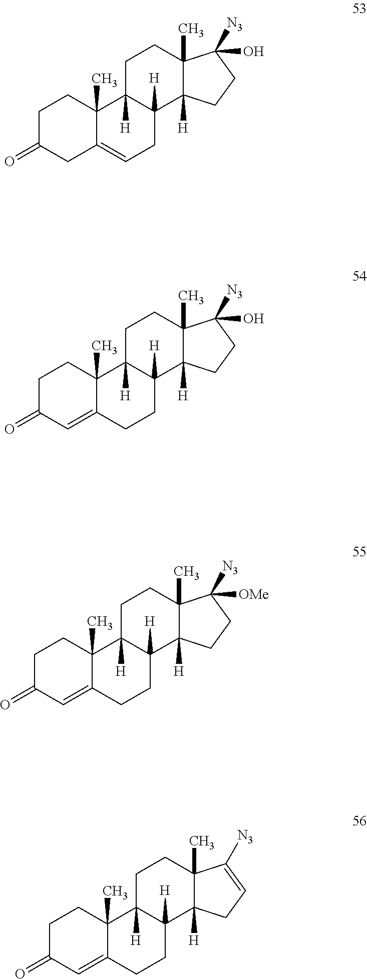

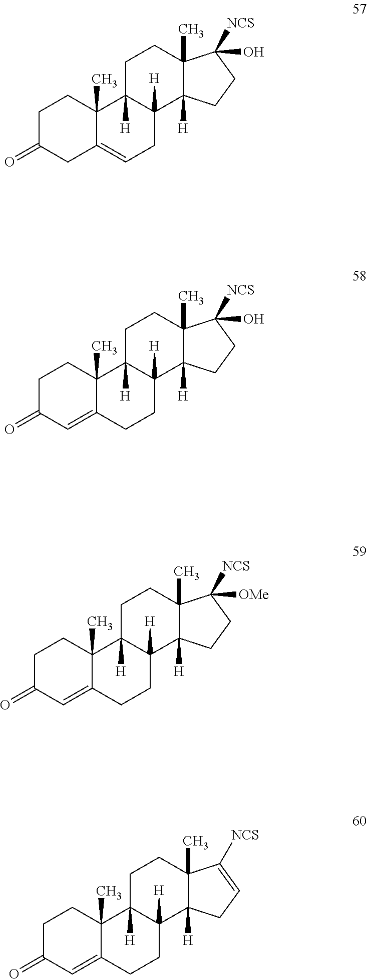

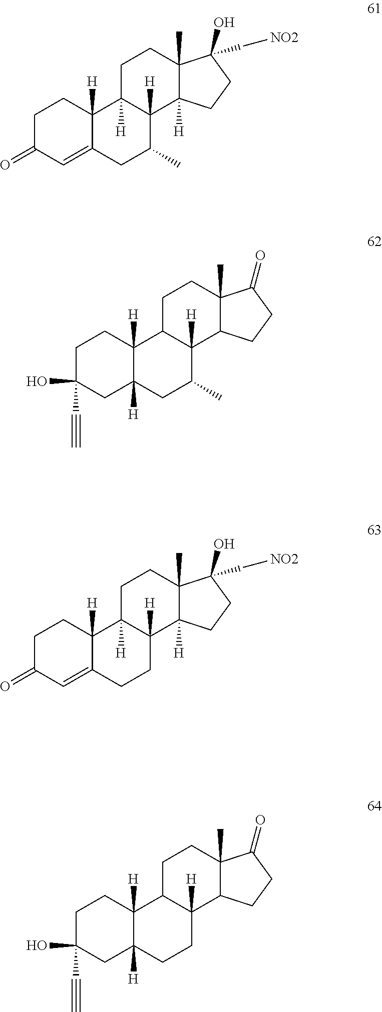

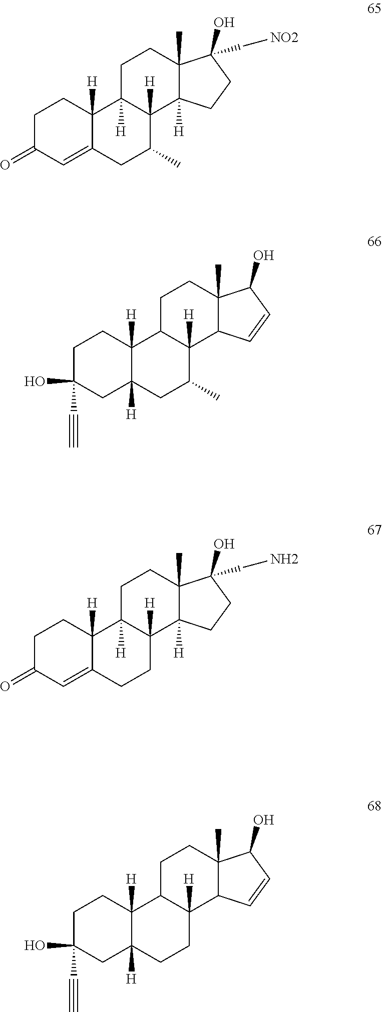

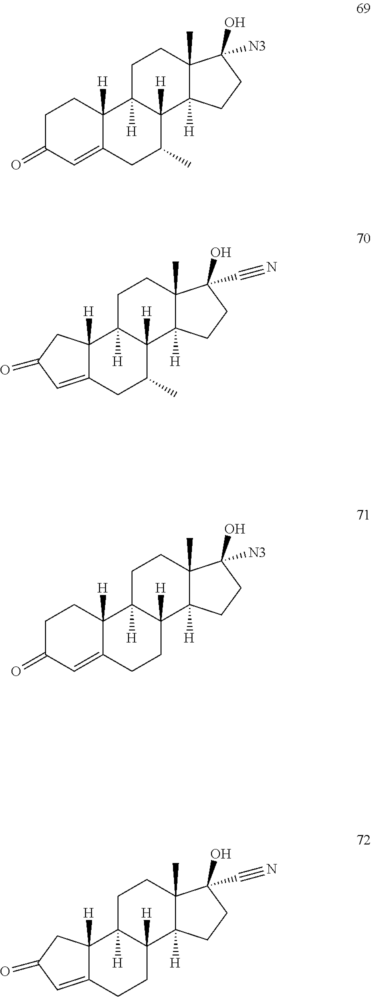

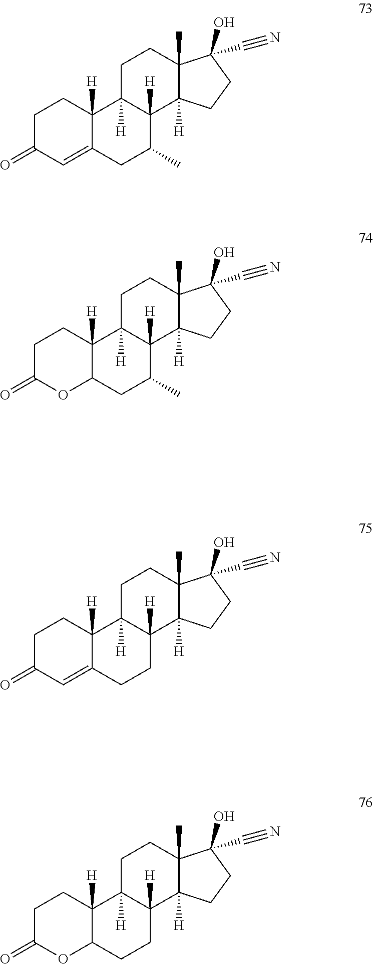

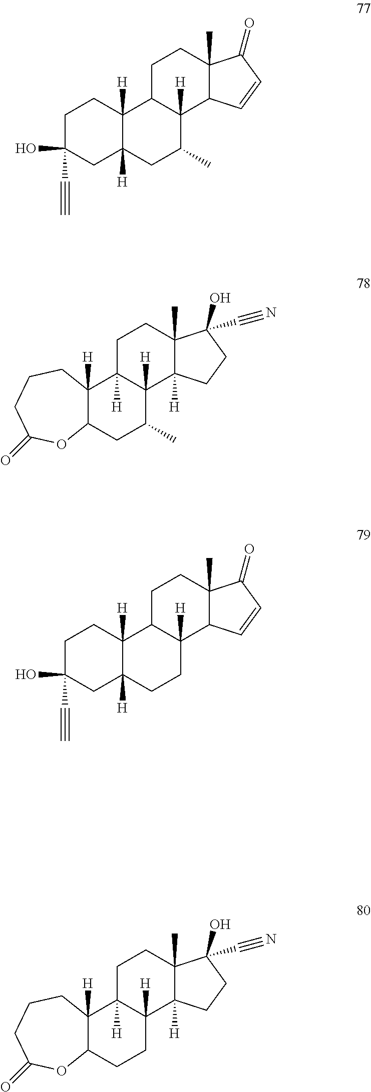

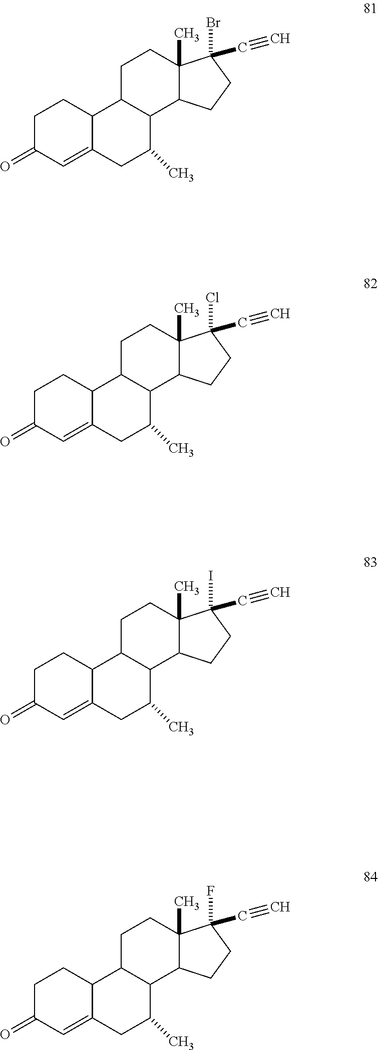 [SMILES: C#C[C@@]1(Br)CCC2C3C(CC[C@@]21C)C1CCC(=O)C=C1C[C@H]3C ; C#C[C@@]1(Cl)CCC2C3C(CC[C@@]21C)C1CCC(=O)C=C1C[C@H]3C ; C#C[C@@]1(I)CCC2C3C(CC[C@@]21C)C1CCC(=O)C=C1C[C@H]3C ; C#C[C@@]1(F)CCC2C3C(CC[C@@]21C)C1CCC(=O)C=C1C[C@H]3C]

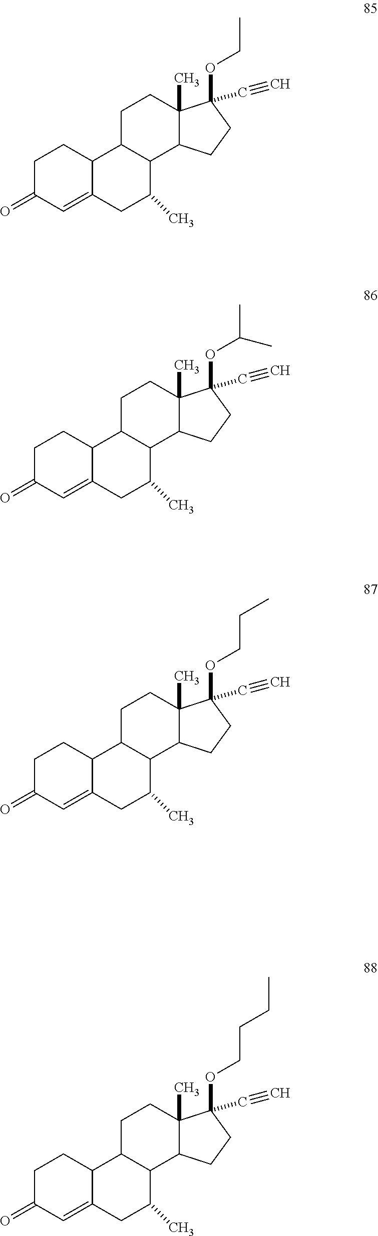 [SMILES: C#C[C@]1(OCC)CCC2C3C(CC[C@@]21C)C1CCC(=O)C=C1C[C@H]3C ; C#C[C@]1(OC(C)C)CCC2C3C(CC[C@@]21C)C1CCC(=O)C=C1C[C@H]3C ; C#C[C@]1(OCCC)CCC2C3C(CC[C@@]21C)C1CCC(=O)C=C1C[C@H]3C ; C#C[C@]1(OCCCC)CCC2C3C(CC[C@@]21C)C1CCC(=O)C=C1C[C@H]3C]

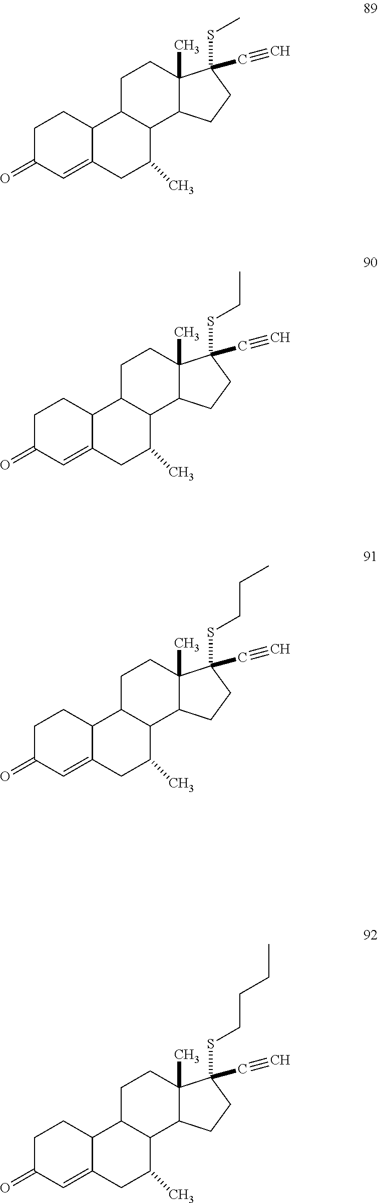 [SMILES: C#C[C@@]1(SC)CCC2C3C(CC[C@@]21C)C1CCC(=O)C=C1C[C@H]3C ; C#C[C@@]1(SCC)CCC2C3C(CC[C@@]21C)C1CCC(=O)C=C1C[C@H]3C ; C#C[C@@]1(SCCC)CCC2C3C(CC[C@@]21C)C1CCC(=O)C=C1C[C@H]3C ; C#C[C@@]1(SCCCC)CCC2C3C(CC[C@@]21C)C1CCC(=O)C=C1C[C@H]3C]

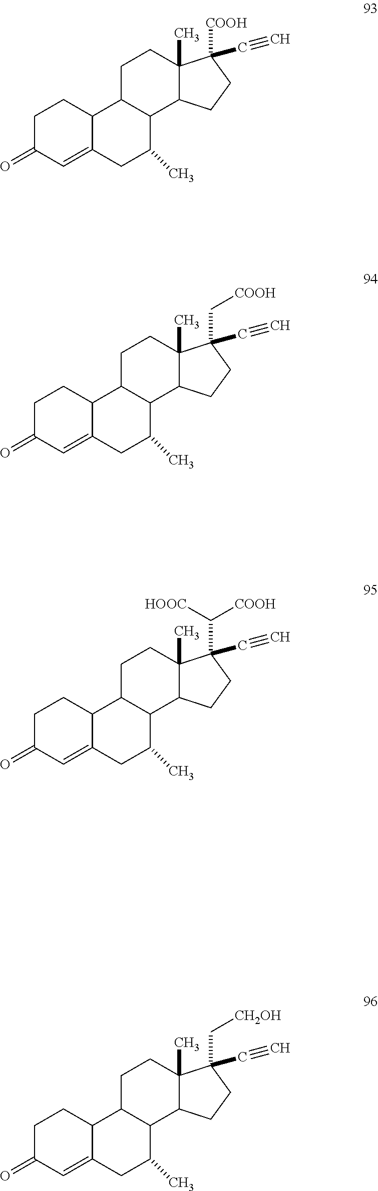 [SMILES: C#C[C@@]1(C(=O)O)CCC2C3C(CC[C@@]21C)C1CCC(=O)C=C1C[C@H]3C ; C#C[C@@]1(CC(=O)O)CCC2C3C(CC[C@@]21C)C1CCC(=O)C=C1C[C@H]3C ; C#C[C@@]1(C(C(=O)O)C(=O)O)CCC2C3C(CC[C@@]21C)C1CCC(=O)C=C1C[C@H]3C ; C#C[C@@]1(CCO)CCC2C3C(CC[C@@]21C)C1CCC(=O)C=C1C[C@H]3C]

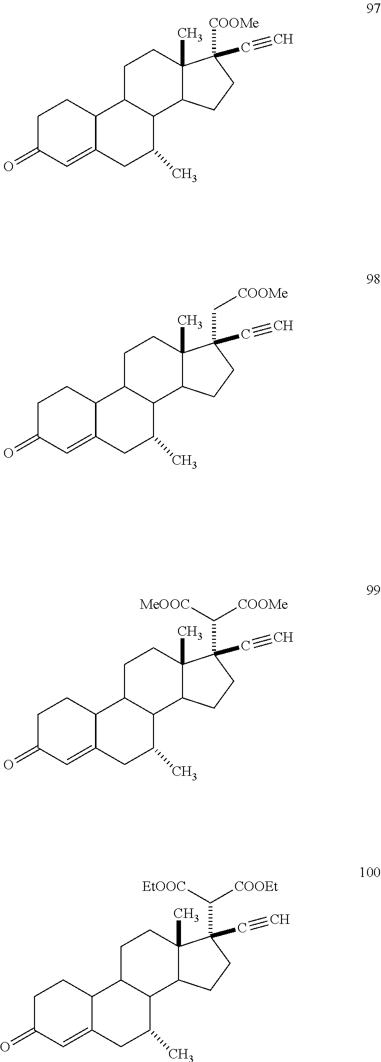 [SMILES: C#C[C@@]1(C(=O)OC)CCC2C3C(CC[C@@]21C)C1CCC(=O)C=C1C[C@H]3C ; C#C[C@@]1(CC(=O)OC)CCC2C3C(CC[C@@]21C)C1CCC(=O)C=C1C[C@H]3C ; C#C[C@@]1(C(C(=O)OC)C(=O)OC)CCC2C3C(CC[C@@]21C)C1CCC(=O)C=C1C[C@H]3C ; C#C[C@@]1(C(C(=O)OCC)C(=O)OCC)CCC2C3C(CC[C@@]21C)C1CCC(=O)C=C1C[C@H]3C]

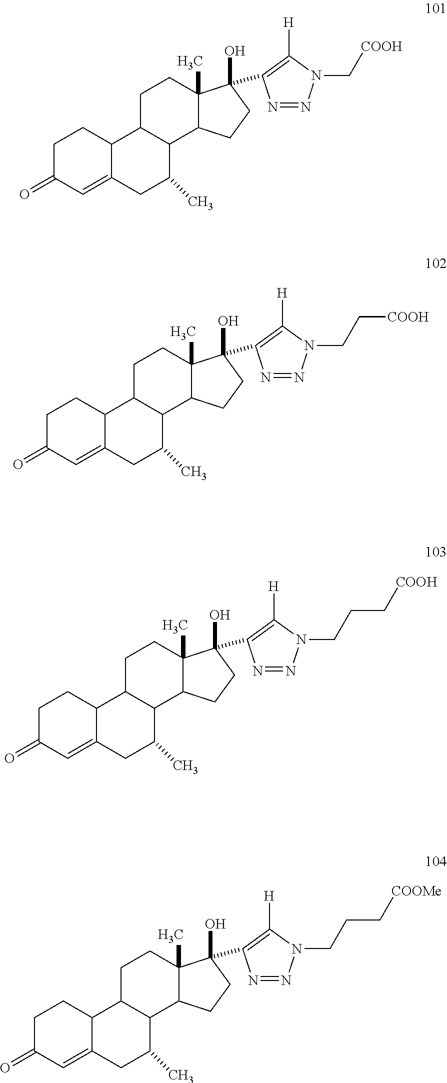 [SMILES: C[C@@H]1CC2=CC(=O)CCC2C2CC[C@@]3(C)C(CC[C@@]3(O)c3cn(CC(=O)O)nn3)C21 ; C[C@@H]1CC2=CC(=O)CCC2C2CC[C@@]3(C)C(CC[C@@]3(O)c3cn(CCC(=O)O)nn3)C21 ; C[C@@H]1CC2=CC(=O)CCC2C2CC[C@@]3(C)C(CC[C@@]3(O)c3cn(CCCC(=O)O)nn3)C21 ; COC(=O)CCCn1cc([C@]2(O)CCC3C4C(CC[C@@]32C)C2CCC(=O)C=C2C[C@H]4C)nn1]

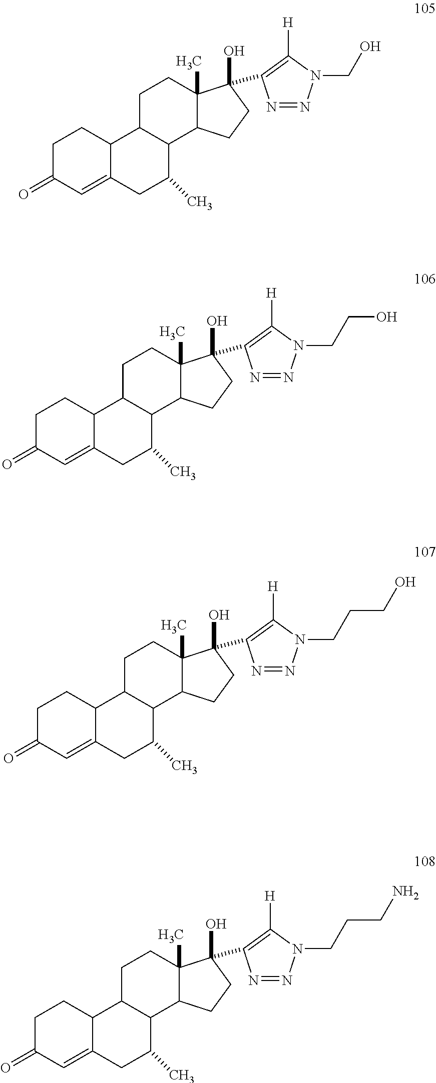 [SMILES: C[C@@H]1CC2=CC(=O)CCC2C2CC[C@@]3(C)C(CC[C@@]3(O)c3cn(CO)nn3)C21 ; C[C@@H]1CC2=CC(=O)CCC2C2CC[C@@]3(C)C(CC[C@@]3(O)c3cn(CCO)nn3)C21 ; C[C@@H]1CC2=CC(=O)CCC2C2CC[C@@]3(C)C(CC[C@@]3(O)c3cn(CCCO)nn3)C21 ; C[C@@H]1CC2=CC(=O)CCC2C2CC[C@@]3(C)C(CC[C@@]3(O)c3cn(CCCN)nn3)C21]

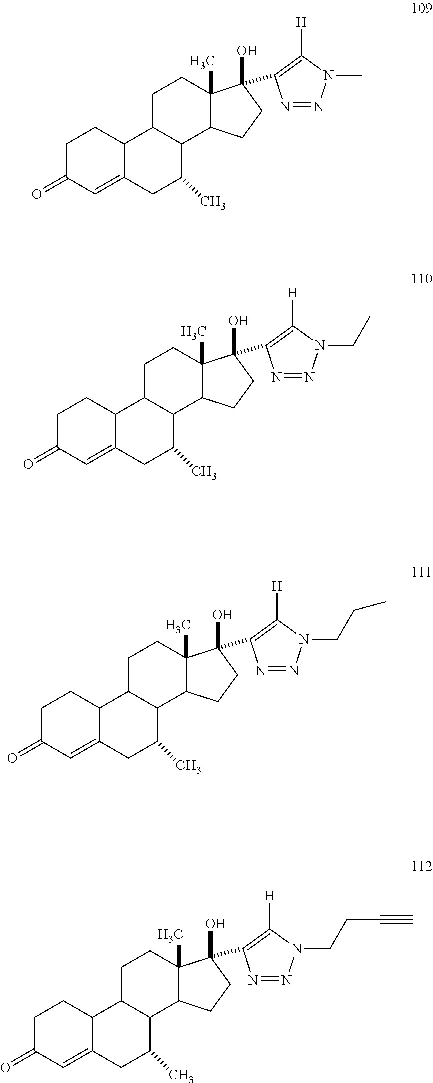 [SMILES: C[C@@H]1CC2=CC(=O)CCC2C2CC[C@@]3(C)C(CC[C@@]3(O)c3cn(C)nn3)C21 ; CCn1cc([C@]2(O)CCC3C4C(CC[C@@]32C)C2CCC(=O)C=C2C[C@H]4C)nn1 ; CCCn1cc([C@]2(O)CCC3C4C(CC[C@@]32C)C2CCC(=O)C=C2C[C@H]4C)nn1 ; C#CCCn1cc([C@]2(O)CCC3C4C(CC[C@@]32C)C2CCC(=O)C=C2C[C@H]4C)nn1]

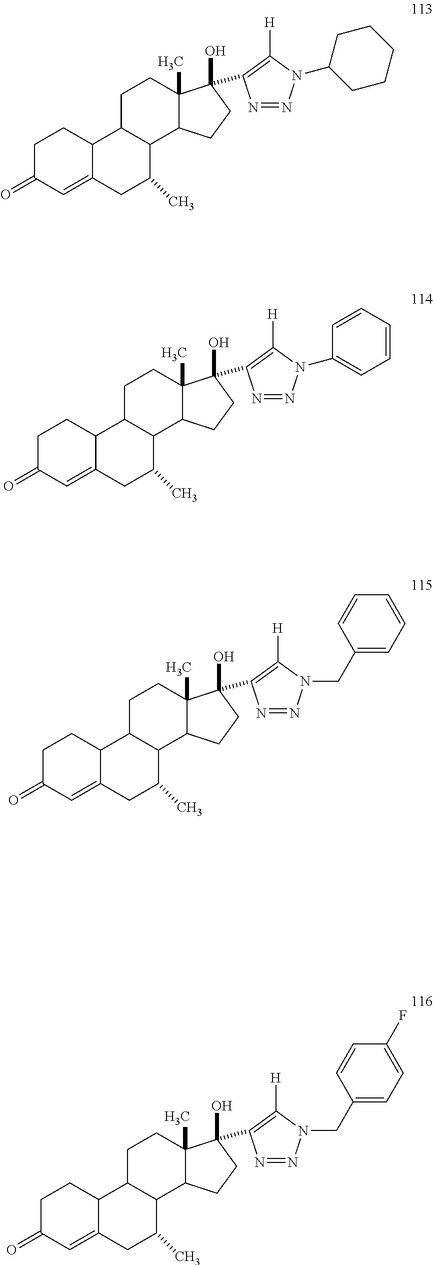 [SMILES: C[C@@H]1CC2=CC(=O)CCC2C2CC[C@@]3(C)C(CC[C@@]3(O)c3cn(C4CCCCC4)nn3)C21 ; C[C@@H]1CC2=CC(=O)CCC2C2CC[C@@]3(C)C(CC[C@@]3(O)c3cn(-c4ccccc4)nn3)C21 ; C[C@@H]1CC2=CC(=O)CCC2C2CC[C@@]3(C)C(CC[C@@]3(O)c3cn(Cc4ccccc4)nn3)C21 ; C[C@@H]1CC2=CC(=O)CCC2C2CC[C@@]3(C)C(CC[C@@]3(O)c3cn(Cc4ccc(F)cc4)nn3)C21]

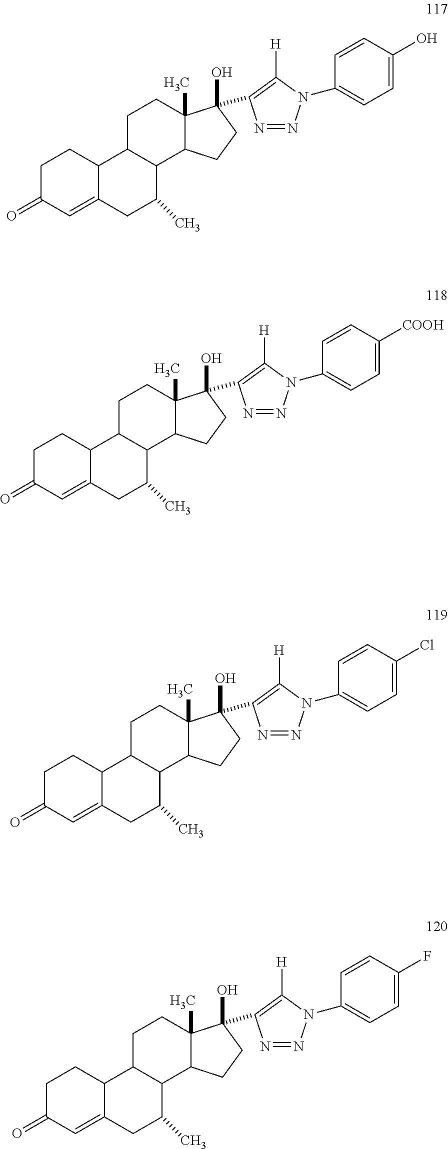 [SMILES: C[C@@H]1CC2=CC(=O)CCC2C2CC[C@@]3(C)C(CC[C@@]3(O)c3cn(-c4ccc(O)cc4)nn3)C21 ; C[C@@H]1CC2=CC(=O)CCC2C2CC[C@@]3(C)C(CC[C@@]3(O)c3cn(-c4ccc(C(=O)O)cc4)nn3)C21 ; C[C@@H]1CC2=CC(=O)CCC2C2CC[C@@]3(C)C(CC[C@@]3(O)c3cn(-c4ccc(Cl)cc4)nn3)C21 ; C[C@@H]1CC2=CC(=O)CCC2C2CC[C@@]3(C)C(CC[C@@]3(O)c3cn(-c4ccc(F)cc4)nn3)C21]

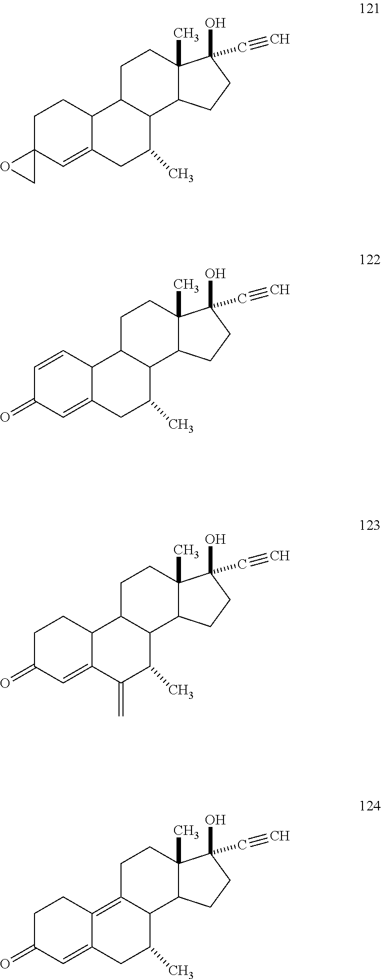 [SMILES: C#C[C@]1(O)CCC2C3C(CC[C@@]21C)C1CCC2(C=C1C[C@H]3C)CO2 ; C#C[C@]1(O)CCC2C3C(CC[C@@]21C)C1C=CC(=O)C=C1C[C@H]3C ; C#C[C@]1(O)CCC2C3C(CC[C@@]21C)C1CCC(=O)C=C1C(=C)[C@H]3C ; C#C[C@]1(O)CCC2C3C(=C4CCC(=O)C=C4C[C@H]3C)CC[C@@]21C]

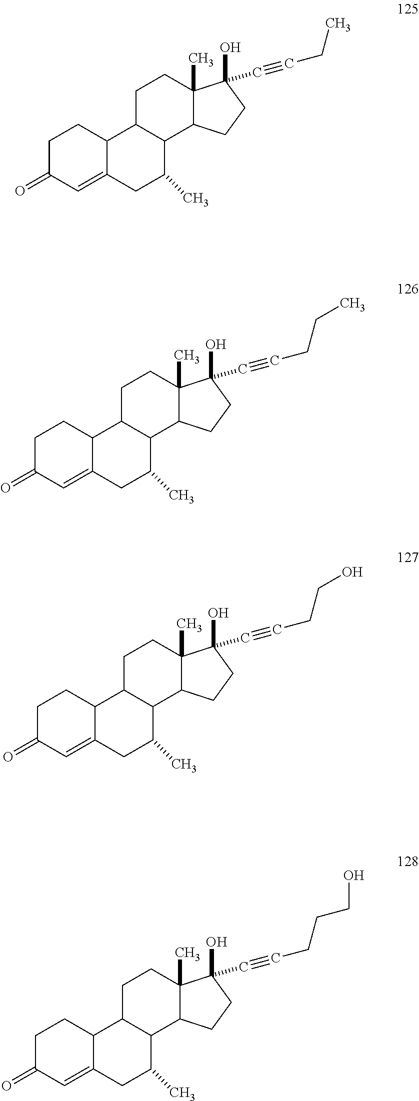 [SMILES: CCC#C[C@]1(O)CCC2C3C(CC[C@@]21C)C1CCC(=O)C=C1C[C@H]3C ; CCCC#C[C@]1(O)CCC2C3C(CC[C@@]21C)C1CCC(=O)C=C1C[C@H]3C ; C[C@@H]1CC2=CC(=O)CCC2C2CC[C@@]3(C)C(CC[C@@]3(O)C#CCCO)C21 ; C[C@@H]1CC2=CC(=O)CCC2C2CC[C@@]3(C)C(CC[C@@]3(O)C#CCCCO)C21]

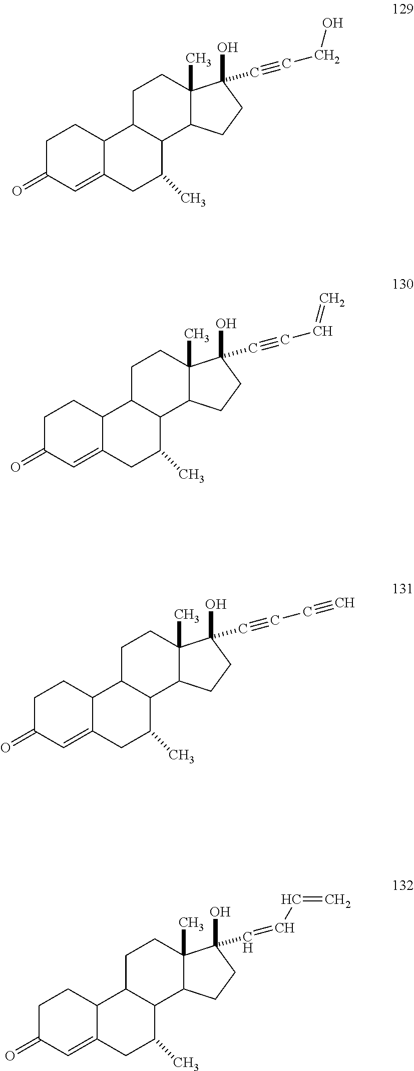 [SMILES: C[C@@H]1CC2=CC(=O)CCC2C2CC[C@@]3(C)C(CC[C@@]3(O)C#CCO)C21 ; C=CC#C[C@]1(O)CCC2C3C(CC[C@@]21C)C1CCC(=O)C=C1C[C@H]3C ; C#CC#C[C@]1(O)CCC2C3C(CC[C@@]21C)C1CCC(=O)C=C1C[C@H]3C ; C=CC=C[C@]1(O)CCC2C3C(CC[C@@]21C)C1CCC(=O)C=C1C[C@H]3C]

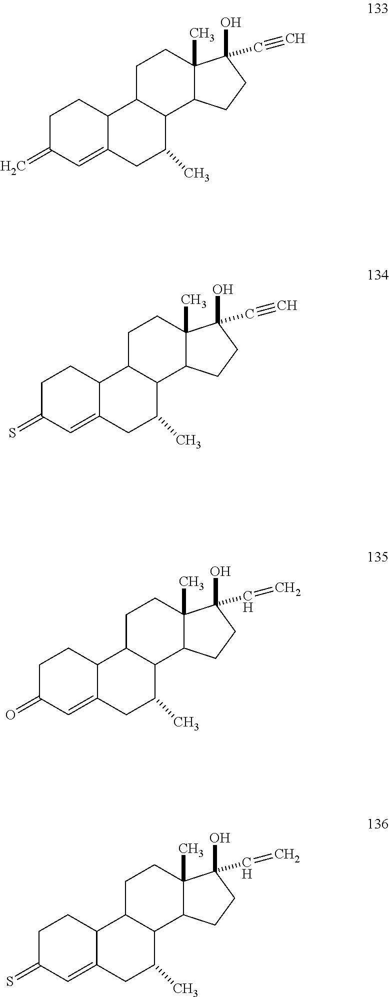 [SMILES: C#C[C@]1(O)CCC2C3C(CC[C@@]21C)C1CCC(=C)C=C1C[C@H]3C ; C#C[C@]1(O)CCC2C3C(CC[C@@]21C)C1CCC(=S)C=C1C[C@H]3C ; C=C[C@]1(O)CCC2C3C(CC[C@@]21C)C1CCC(=O)C=C1C[C@H]3C ; C=C[C@]1(O)CCC2C3C(CC[C@@]21C)C1CCC(=S)C=C1C[C@H]3C]

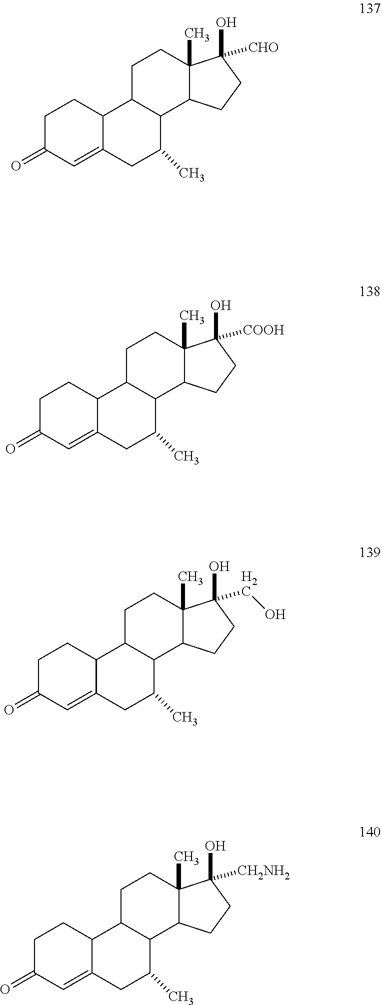

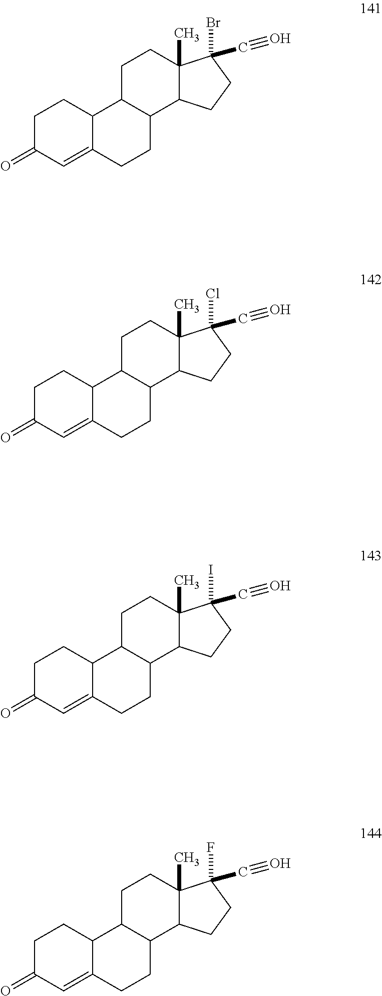

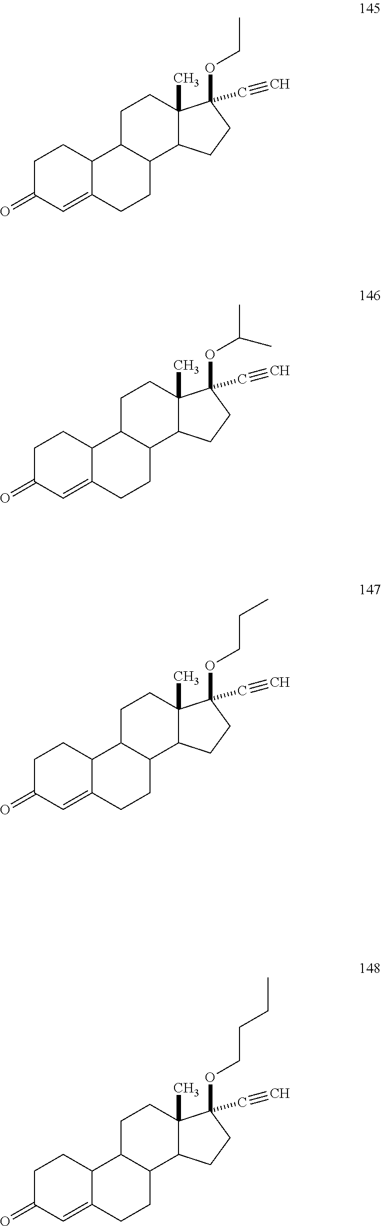 [SMILES: C#C[C@]1(OCC)CCC2C3CCC4=CC(=O)CCC4C3CC[C@@]21C ; C#C[C@]1(OC(C)C)CCC2C3CCC4=CC(=O)CCC4C3CC[C@@]21C ; C#C[C@]1(OCCC)CCC2C3CCC4=CC(=O)CCC4C3CC[C@@]21C ; C#C[C@]1(OCCCC)CCC2C3CCC4=CC(=O)CCC4C3CC[C@@]21C]

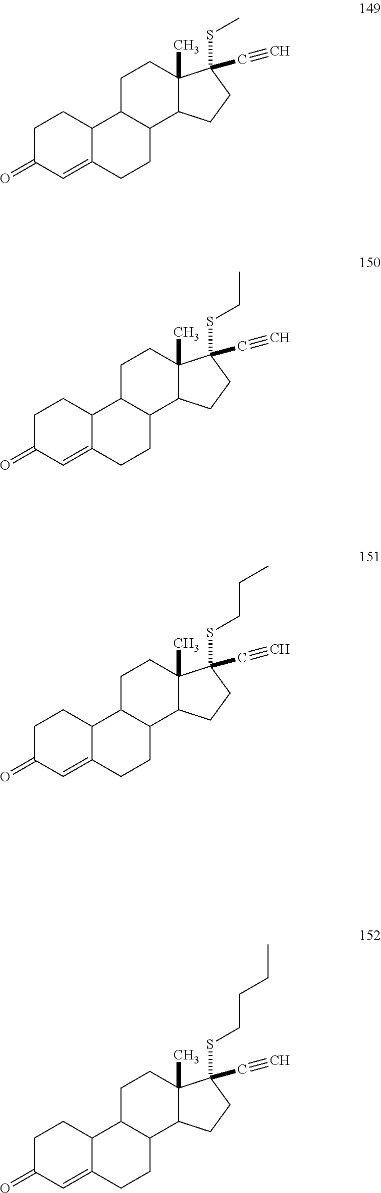 [SMILES: C#C[C@@]1(SC)CCC2C3CCC4=CC(=O)CCC4C3CC[C@@]21C ; C#C[C@@]1(SCC)CCC2C3CCC4=CC(=O)CCC4C3CC[C@@]21C ; C#C[C@@]1(SCCC)CCC2C3CCC4=CC(=O)CCC4C3CC[C@@]21C ; C#C[C@@]1(SCCCC)CCC2C3CCC4=CC(=O)CCC4C3CC[C@@]21C]

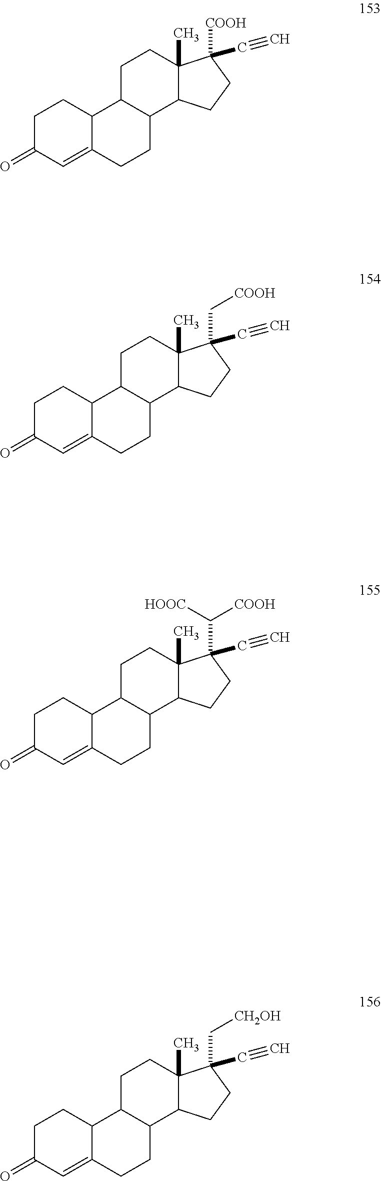 [SMILES: C#C[C@@]1(C(=O)O)CCC2C3CCC4=CC(=O)CCC4C3CC[C@@]21C ; C#C[C@@]1(CC(=O)O)CCC2C3CCC4=CC(=O)CCC4C3CC[C@@]21C ; C#C[C@@]1(C(C(=O)O)C(=O)O)CCC2C3CCC4=CC(=O)CCC4C3CC[C@@]21C ; C#C[C@@]1(CCO)CCC2C3CCC4=CC(=O)CCC4C3CC[C@@]21C]

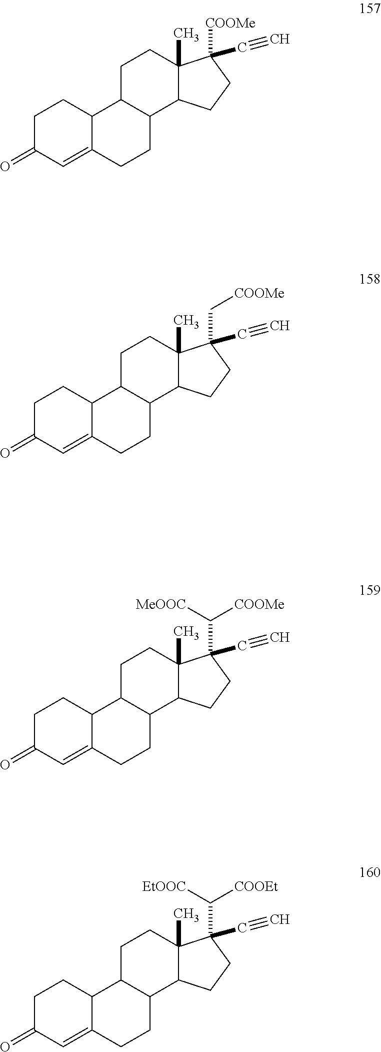 [SMILES: C#C[C@@]1(C(=O)OC)CCC2C3CCC4=CC(=O)CCC4C3CC[C@@]21C ; C#C[C@@]1(CC(=O)OC)CCC2C3CCC4=CC(=O)CCC4C3CC[C@@]21C ; C#C[C@@]1(C(C(=O)OC)C(=O)OC)CCC2C3CCC4=CC(=O)CCC4C3CC[C@@]21C ; C#C[C@@]1(C(C(=O)OCC)C(=O)OCC)CCC2C3CCC4=CC(=O)CCC4C3CC[C@@]21C]

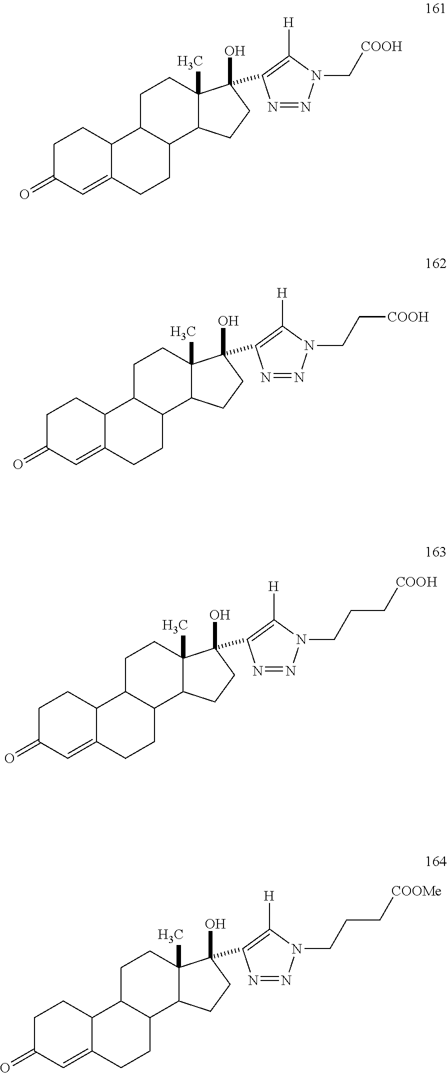 [SMILES: C[C@]12CCC3C4CCC(=O)C=C4CCC3C1CC[C@@]2(O)c1cn(CC(=O)O)nn1 ; C[C@]12CCC3C4CCC(=O)C=C4CCC3C1CC[C@@]2(O)c1cn(CCC(=O)O)nn1 ; C[C@]12CCC3C4CCC(=O)C=C4CCC3C1CC[C@@]2(O)c1cn(CCCC(=O)O)nn1 ; COC(=O)CCCn1cc([C@]2(O)CCC3C4CCC5=CC(=O)CCC5C4CC[C@@]32C)nn1]

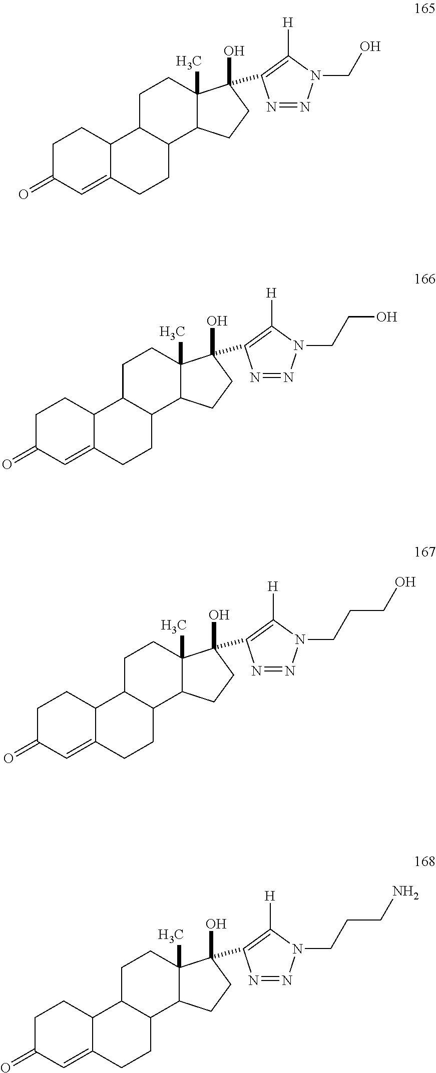 [SMILES: C[C@]12CCC3C4CCC(=O)C=C4CCC3C1CC[C@@]2(O)c1cn(CO)nn1 ; C[C@]12CCC3C4CCC(=O)C=C4CCC3C1CC[C@@]2(O)c1cn(CCO)nn1 ; C[C@]12CCC3C4CCC(=O)C=C4CCC3C1CC[C@@]2(O)c1cn(CCCO)nn1 ; C[C@]12CCC3C4CCC(=O)C=C4CCC3C1CC[C@@]2(O)c1cn(CCCN)nn1]

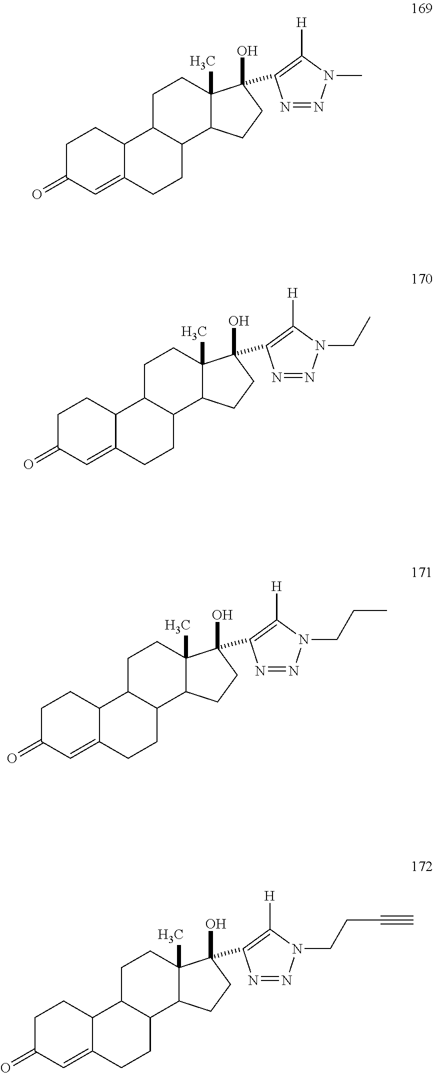 [SMILES: Cn1cc([C@]2(O)CCC3C4CCC5=CC(=O)CCC5C4CC[C@@]32C)nn1 ; CCn1cc([C@]2(O)CCC3C4CCC5=CC(=O)CCC5C4CC[C@@]32C)nn1 ; CCCn1cc([C@]2(O)CCC3C4CCC5=CC(=O)CCC5C4CC[C@@]32C)nn1 ; C#CCCn1cc([C@]2(O)CCC3C4CCC5=CC(=O)CCC5C4CC[C@@]32C)nn1]

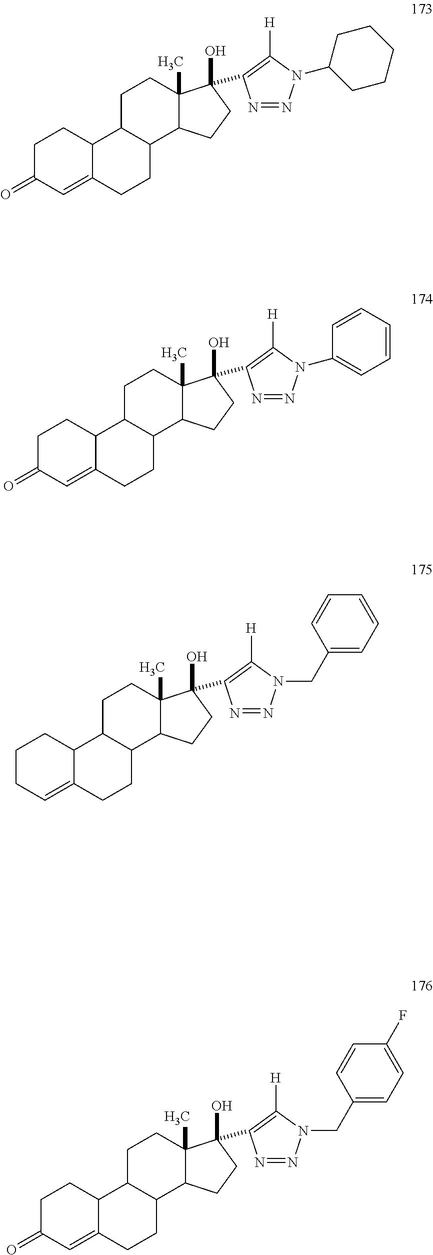 [SMILES: C[C@]12CCC3C4CCC(=O)C=C4CCC3C1CC[C@@]2(O)c1cn(C2CCCCC2)nn1 ; C[C@]12CCC3C4CCC(=O)C=C4CCC3C1CC[C@@]2(O)c1cn(-c2ccccc2)nn1 ; C[C@]12CCC3C4CCCC=C4CCC3C1CC[C@@]2(O)c1cn(Cc2ccccc2)nn1 ; C[C@]12CCC3C4CCC(=O)C=C4CCC3C1CC[C@@]2(O)c1cn(Cc2ccc(F)cc2)nn1]

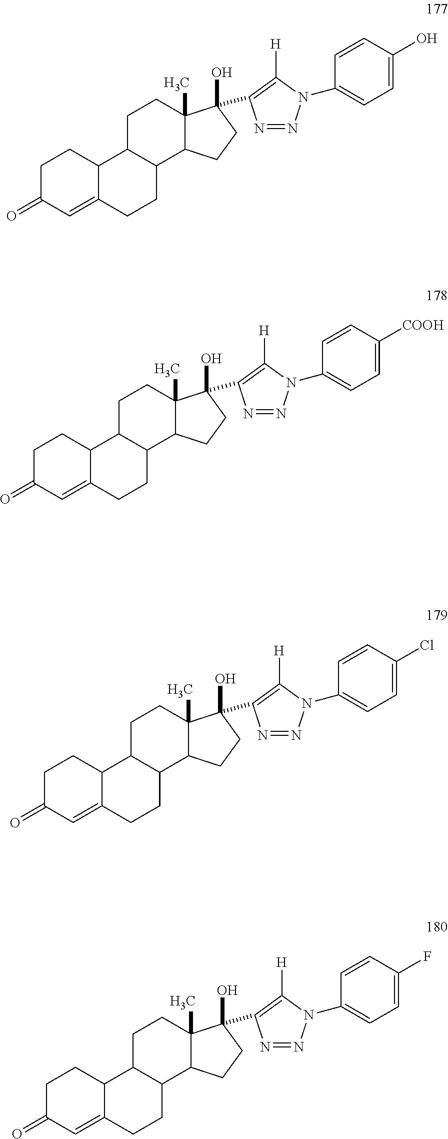 [SMILES: C[C@]12CCC3C4CCC(=O)C=C4CCC3C1CC[C@@]2(O)c1cn(-c2ccc(O)cc2)nn1 ; C[C@]12CCC3C4CCC(=O)C=C4CCC3C1CC[C@@]2(O)c1cn(-c2ccc(C(=O)O)cc2)nn1 ; C[C@]12CCC3C4CCC(=O)C=C4CCC3C1CC[C@@]2(O)c1cn(-c2ccc(Cl)cc2)nn1 ; C[C@]12CCC3C4CCC(=O)C=C4CCC3C1CC[C@@]2(O)c1cn(-c2ccc(F)cc2)nn1]

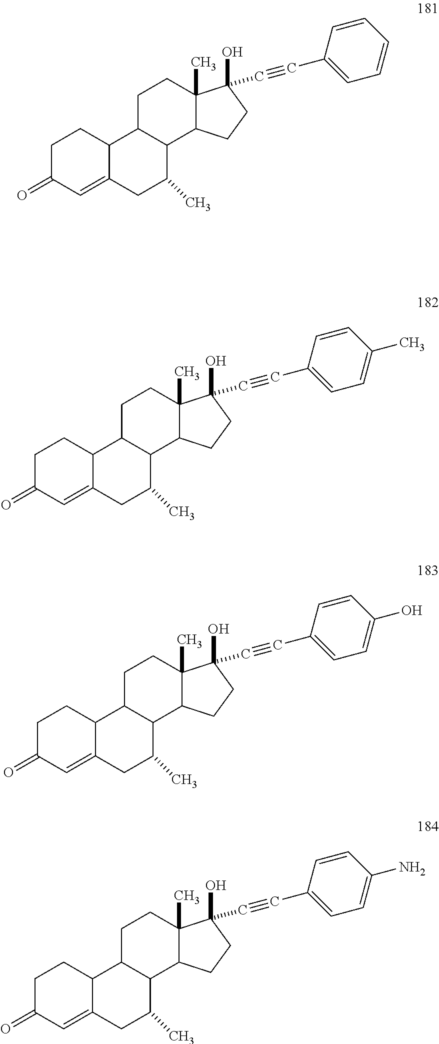 [SMILES: C[C@@H]1CC2=CC(=O)CCC2C2CC[C@@]3(C)C(CC[C@@]3(O)C#Cc3ccccc3)C21 ; Cc1ccc(C#C[C@]2(O)CCC3C4C(CC[C@@]32C)C2CCC(=O)C=C2C[C@H]4C)cc1 ; C[C@@H]1CC2=CC(=O)CCC2C2CC[C@@]3(C)C(CC[C@@]3(O)C#Cc3ccc(O)cc3)C21 ; C[C@@H]1CC2=CC(=O)CCC2C2CC[C@@]3(C)C(CC[C@@]3(O)C#Cc3ccc(N)cc3)C21]

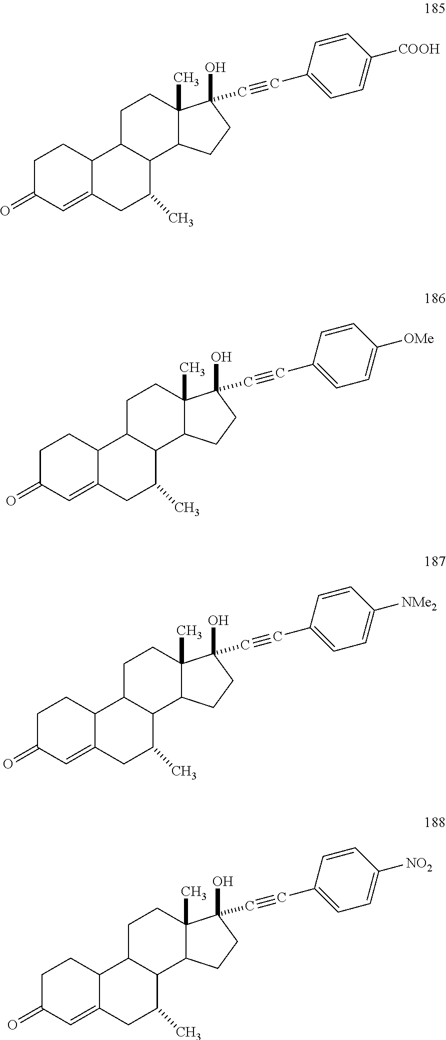 [SMILES: C[C@@H]1CC2=CC(=O)CCC2C2CC[C@@]3(C)C(CC[C@@]3(O)C#Cc3ccc(C(=O)O)cc3)C21 ; COc1ccc(C#C[C@]2(O)CCC3C4C(CC[C@@]32C)C2CCC(=O)C=C2C[C@H]4C)cc1 ; C[C@@H]1CC2=CC(=O)CCC2C2CC[C@@]3(C)C(CC[C@@]3(O)C#Cc3ccc(N(C)C)cc3)C21 ; C[C@@H]1CC2=CC(=O)CCC2C2CC[C@@]3(C)C(CC[C@@]3(O)C#Cc3ccc([N+](=O)[O-])cc3)C21]

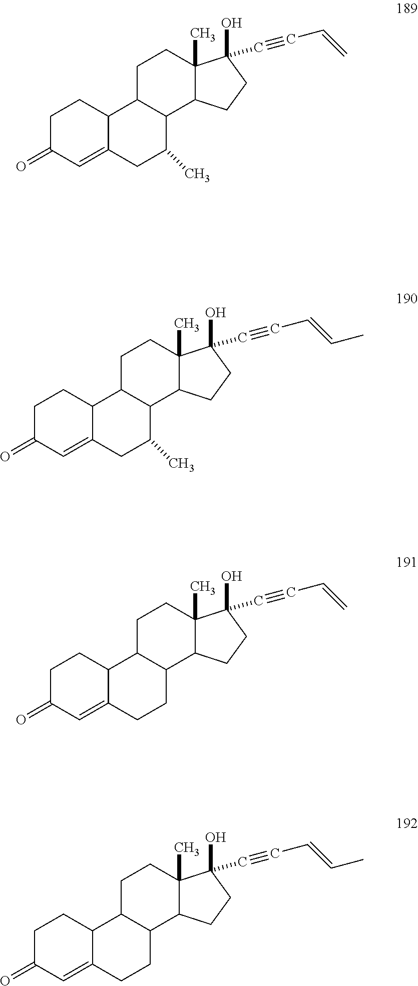 [SMILES: C=CC#C[C@]1(O)CCC2C3C(CC[C@@]21C)C1CCC(=O)C=C1C[C@H]3C ; C/C=C/C#C[C@]1(O)CCC2C3C(CC[C@@]21C)C1CCC(=O)C=C1C[C@H]3C ; C=CC#C[C@]1(O)CCC2C3CCC4=CC(=O)CCC4C3CC[C@@]21C ; C/C=C/C#C[C@]1(O)CCC2C3CCC4=CC(=O)CCC4C3CC[C@@]21C]

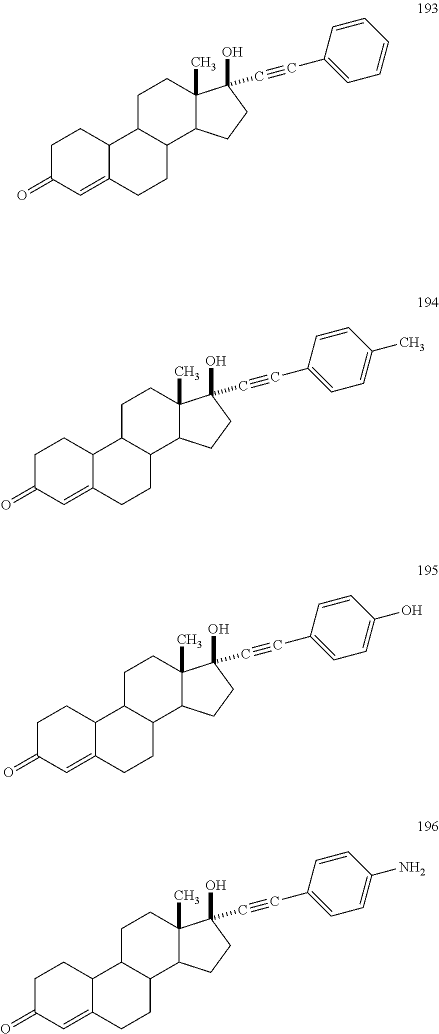 [SMILES: C[C@]12CCC3C4CCC(=O)C=C4CCC3C1CC[C@@]2(O)C#Cc1ccccc1 ; Cc1ccc(C#C[C@]2(O)CCC3C4CCC5=CC(=O)CCC5C4CC[C@@]32C)cc1 ; C[C@]12CCC3C4CCC(=O)C=C4CCC3C1CC[C@@]2(O)C#Cc1ccc(O)cc1 ; C[C@]12CCC3C4CCC(=O)C=C4CCC3C1CC[C@@]2(O)C#Cc1ccc(N)cc1]

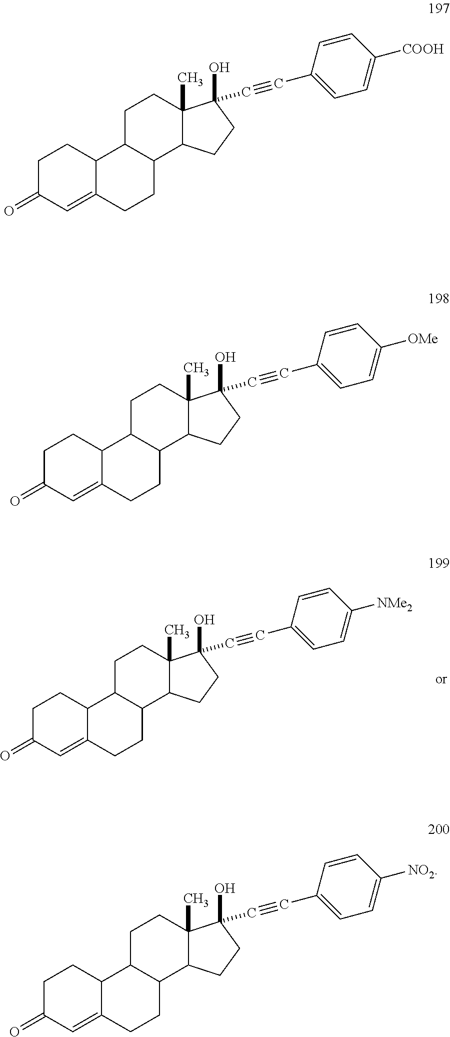 [SMILES: C[C@]12CCC3C4CCC(=O)C=C4CCC3C1CC[C@@]2(O)C#Cc1ccc(C(=O)O)cc1 ; COc1ccc(C#C[C@]2(O)CCC3C4CCC5=CC(=O)CCC5C4CC[C@@]32C)cc1 ; CN(C)c1ccc(C#C[C@]2(O)CCC3C4CCC5=CC(=O)CCC5C4CC[C@@]32C)cc1 ; C[C@]12CCC3C4CCC(=O)C=C4CCC3C1CC[C@@]2(O)C#Cc1ccc([N+](=O)[O-])cc1]

In some illustrative embodiments, the present invention relates to a pharmaceutical composition comprising one or more compounds as disclosed herein, or a pharmaceutically acceptable salt thereof, together with one or more diluents, excipients or carriers.

In some illustrative embodiments, the present invention relates to one or more compounds as disclosed herein, wherein the compound is for the treatment of a cancer.

In some illustrative embodiments, the present invention relates to one or more compounds as disclosed herein, wherein the compound is for the treatment of a cancer.

In some illustrative embodiments, the present invention relates to one or more compounds as disclosed herein, wherein the compound is for the treatment of castration resistant prostate cancer.

In some illustrative embodiments, the present invention relates to a method for treating a cancer patient, comprising the step of administering a therapeutically effective amount of one or more compounds as disclosed herein, and one or more carriers, diluents, or excipients, to a patient in need of relief from said cancer.

In some illustrative embodiments, the present invention relates to a method for treating a cancer patient, comprising the step of administering a therapeutically effective amount of one or more compounds as disclosed herein, and one or more carriers, diluents, or excipients, to a patient in need of relief from said castration resistant prostate cancer.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said cancer.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said castration resistant prostate cancer.

In some illustrative embodiments, the present invention relates to a pharmaceutical composition comprising one or more compounds as disclosed herein, or a pharmaceutically acceptable salt thereof, together with one or more diluents, excipients or carriers, for use as a medicament for cancer.

In some illustrative embodiments, the present invention relates to a method for treating a cancer patient, comprising the step of administering a therapeutically effective amount of one or more compounds, together with one or more carriers, diluents, or excipients, to a patient in need of relief from said cancer, the compound having the formula:

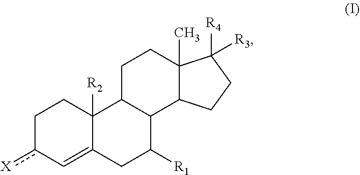

or a pharmaceutically acceptable salt thereof, wherein
[symbol] represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, amino alkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, amino alkyl, heteroalkyl, heteroalkenyl, heteroalkynyl, heterocyclyl, cycloalkyl, cyclo alkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a method for treating a cancer patient, comprising the step of administering a therapeutically effective amount of one or more compounds as disclosed herein in combination with one or more other compounds of the same or different mode of action, and one or more carriers, diluents, or excipients, to a patient in need of relief from said cancer is castration resistant prostate cancer.

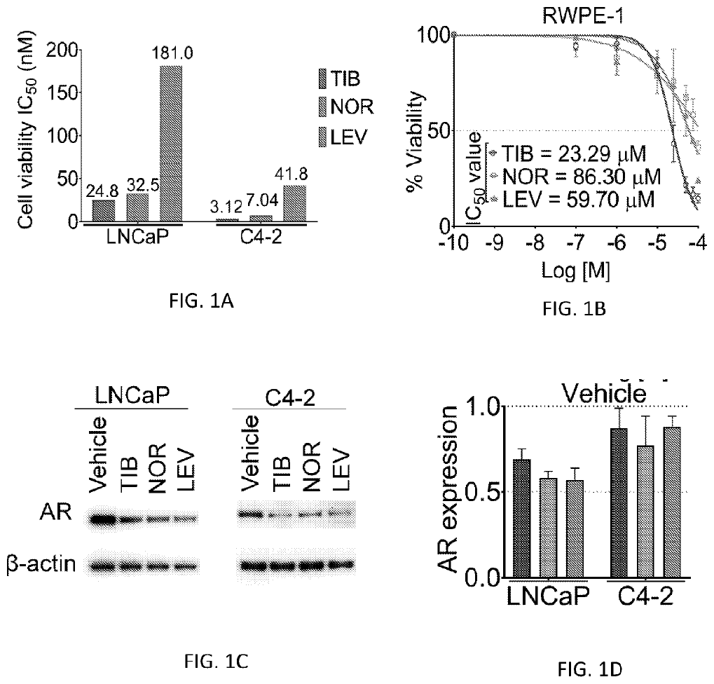

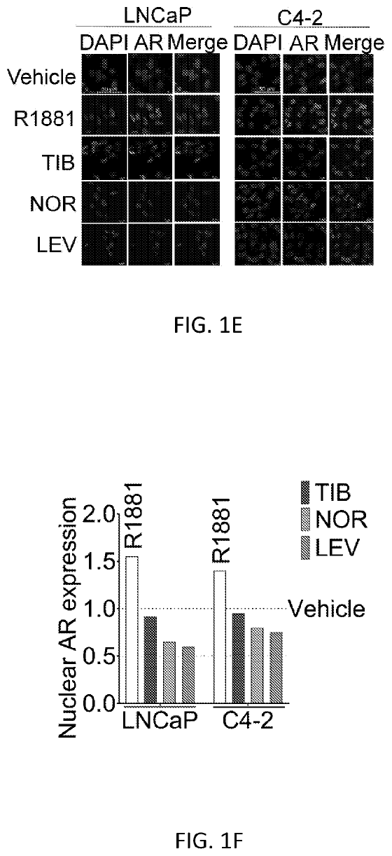

Using CANDO computational tool, we have discovered a set of ten human approved drugs namely azaperone (AZA), buspirone (BUS), cinnarizine (CIN), talampicillin (TAL), pipamperone (PIP), cetraxate (CET), didanosine (DID), tibolone (TIB), norethisterone (NOR) and levonorgestrel (LEV) which interact with a subset of targets that are known to be involved or overexpressed in castration-resistant prostate cancer (CRPC). To identify initial lead(s), we first tested all ten drugs in vitro in human prostate cancer LNCaP and CRPC C4-2 cells to see their growth inhibition effect. Among these ten predicted drugs, TIB, NOR and LEV displayed promising growth inhibition of both LNCaP and C4-2 cells with an IC50 of 24.86, 32.52 and 181.0 nM respectively in LNCaP cells and 3.12, 7.04 and 41.78 nM in C4-2 cells, while for the other drugs this IC50 value was more than 5.0 μM (FIG. 1a). Encouraged by the significant inhibition of CRPC C4-2 cell proliferation by these three drugs, we were enthusiastic to see their cytotoxic effect on the growth of normal human prostatic epithelial RWPE-1 cells. The cytotoxicity IC50 of TIB, NOR and LEV were found to be 23.29, 86.30 and 59.70 μM respectively (FIG. 1b). Because of the known implication of androgen receptor (AR) in CRPC, we therefore investigated whether these initial leads can induce degradation of AR itself in LNCaP and C4-2 cells.

In order to see whole cell expression of AR, we performed western blot analysis of LNCaP and C4-2 cells' lysates after treated with the indicated drugs/compounds (FIGS. 1C-1D). Western blot results revealed that all these leads degraded the AR in LNCaP cells to an extent of 25-45% as compared to that of the vehicle treatment. While in C4-2 cells, this degradation was 15-25% as compared to that of the vehicle treatment. Next, we were interested to see how these leads cause degradation of nuclear AR expression. To understand this, we performed anti-AR-immunofluorescence staining of the LNCaP and C4-2 cells after fixation and permeabilization with Triton X-100, followed by probed with both monoclonal and polyclonal antibodies to AR. (FIG. 1E-1F). Except TIB, both NOR and LEV treatment led to 60% and 30% decrease in nuclear AR level in LNCaP and C4-2 cells respectively—in a fashion that mimics their western blot results. Our findings revealed that TBI, NOR and LEV which are commonly used drugs for hormone replacement therapy, hormonal contraceptive and birth control treatments respectively, have significant inhibitory effect on proliferation of CRPC cells.

Using both the scaffolds and functionalities of the parent leads TIB, NOR and LEV (shown in FIG. 2A), we designed and synthesized a library of new small molecules (1-40, FIG. 2B). This library of design was made through variation of substituents or groups on the C3-keto, C17 hydroxyl and C17 ethynyl groups of the parent leads. Well established oxidation, reduction, alkylation, C—C bond coupling etc. reactions were employed to synthesize these selected molecules. Sonogashira coupling reaction was employed to install aryl functionality through C—C bonds and alkylation of some of these molecules was achieved by reacting alkyl halide in presence of base such as triethyl amine, sodium hydride etc.

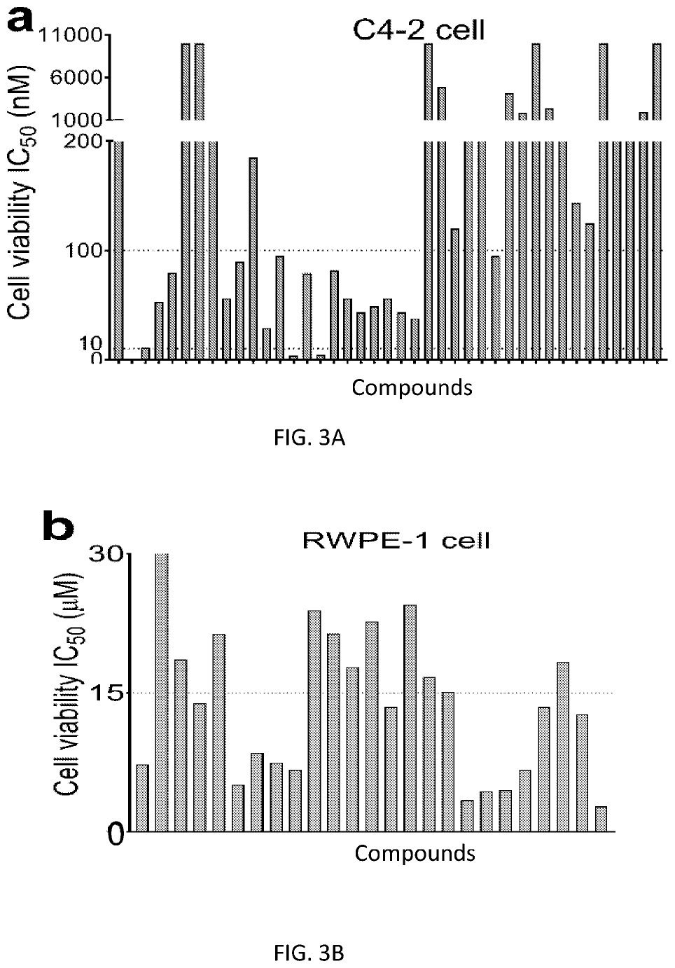

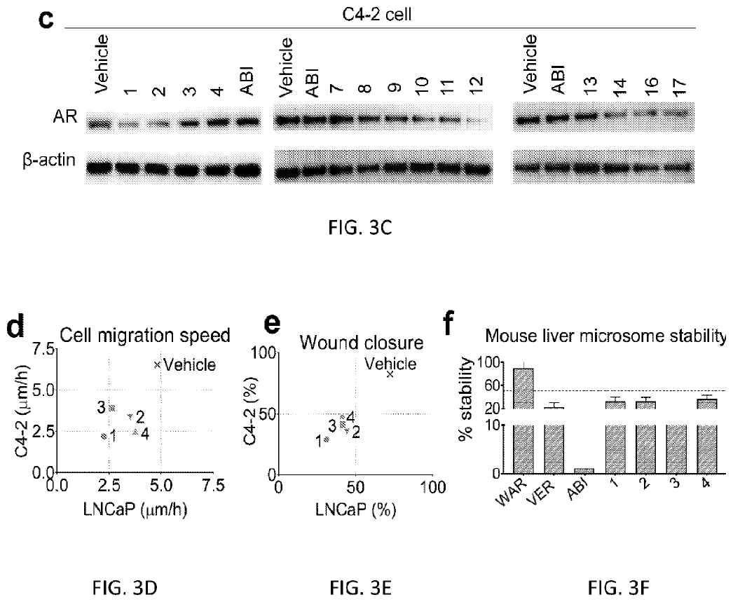

We assessed anti-cancer activity of the synthetic molecules in prostate cancer cell lines as well as toxicity of the active molecules in normal human cell line. We tested all the synthetic compounds 1-40 in C4-2 cells to see their anti-cancer activity. The results revealed that compounds 1, 2, 13 and 15 inhibited the proliferation of C4-2 cells with an IC50 less than 10 nM. (IC50 0.72, 11.01, 3.5 and 4.1 nM for 1, 2, 13 and 15 respectively); while IC50 of compounds 3, 4, 8, 9, 11, 12, 14 and 16-22 was below 100 nM and rest of the compounds were inactive with IC50 greater than 5.0 μM in C4-2 cells (FIG. 3A).

We were also interested to see the potency of our most potent compound 1 against the current steroidal CRPC drug namely abiraterone (ABI, potent CYP17A1 inhibitor). The proliferation IC50 of 1 was found to be much less in C4-2 cells than that of ABI. The cytotoxicity IC50 of these active molecules was found to be 54.6, 18.3, 17.8 and 13.5 μM in RWPE-1 cells for 1, 2, 13 and 15 respectively. The cytotoxicity IC50 of 1 was found to be much higher than that of the ABI (7.20 μM) in RWPE-1 cells (FIG. 3b). To determine whether these active and non-toxic synthetic compounds can induce degradation of AR, we performed western blot analysis of C4-2 cells' lysates after treated with the indicated drugs/compounds (FIG. 3c). Western blot results revealed that synthetic compound 1 degraded AR efficiently in C4-2 cells as compared to that of the vehicle and other treatments including known CRPC drug ABI.

We evaluated effects of the active leads on the cancer cell migration—as most often, prostate cancer cell metastasizes to bone leading to the advanced prostate cancer. We performed the well developed in vitro scratch assay to measure both LNCaP and C4-2 cell's migration in presence of the treatments of all these active synthetic compounds. We calculated the migration speed based on the distance travelled/h. The LNCaP cells with no treatment migrated at a speed of 4.8 μm/h, whereas, treatment with 1, 2, 3 and 4 led to a slower rate (2.2, 3.5, 2.6 and 3.7 μm/h respectively) of migration; almost half of the migration speed as compared to that of the non-treated cells. The C4-2 cells with no treatment migrated at a speed of 6.5 μm/h whereas, treatment with 1, 2, 3 and 4 led to a slower rate (2.4, 3.3, 3.9 and 2.5 μm/h respectively) of migration (FIGS. 3D-3E). Since migration is the first step towards invasion and metastasis of cancer cells 1, 2, 3 and 4 might as well interfere with the metastasis process and lead 1 was found as the most efficient among these based on the migration speed rate in both the cells.

We assessed metabolic stability of the active compounds in presence of both mouse liver microsomes. In this assay, the lead compounds were incubated with the respective microsomes at 37° C. and the incubated mixture was analyzed by LC-MS/MS to quantify the remaining parent molecule. The data are shown in FIG. 3F. Our most potent compound 1 and other active leads were more stable than the current CRPC drug ABI in mouse liver microsome assay.

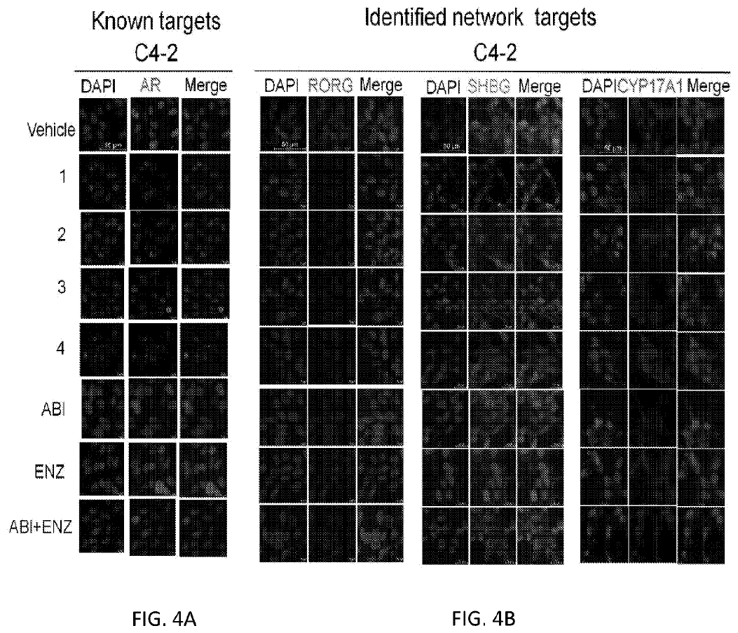

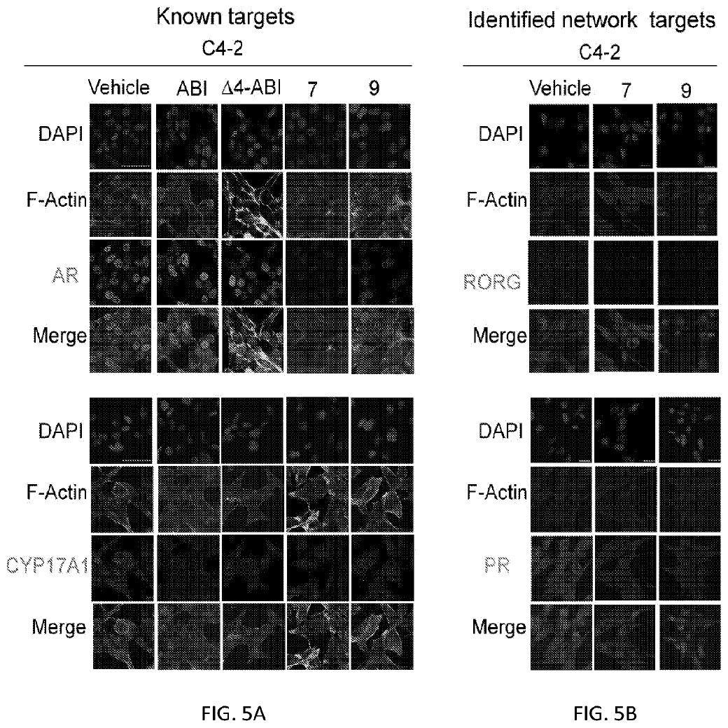

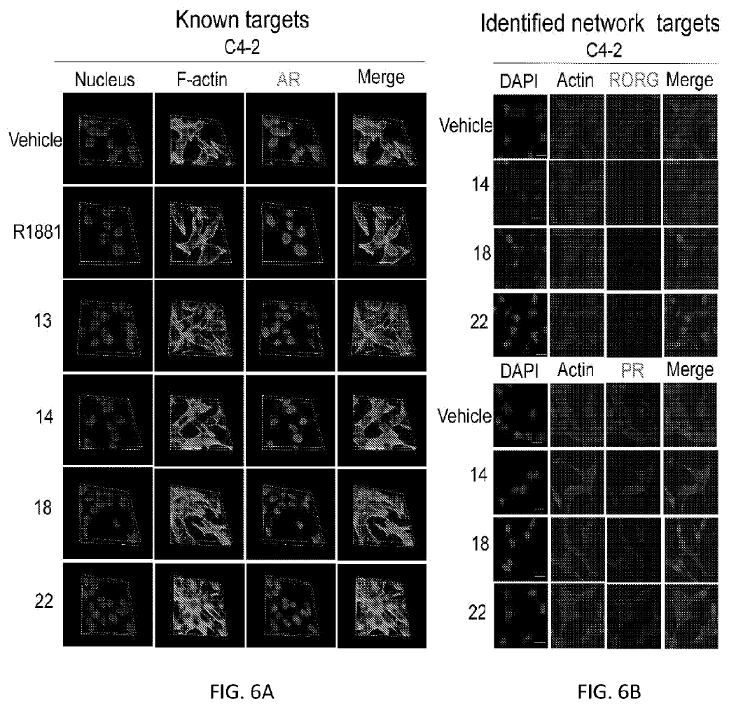

Next, we were interested to see how these active leads cause degradation of known and identified proteome targets. We performed anti-AR immunofluorescent staining of the C4-2 cells after fixation and permeabilization with Triton X-100, followed by probed with both monoclonal and polyclonal antibodies to protein targets. Results are summarized in FIG. 4 (for lead compounds 1-4), FIG. 5 (for lead compounds 7 and 9) and FIG. 6 (for lead compounds 13, 14, 18 and 22).

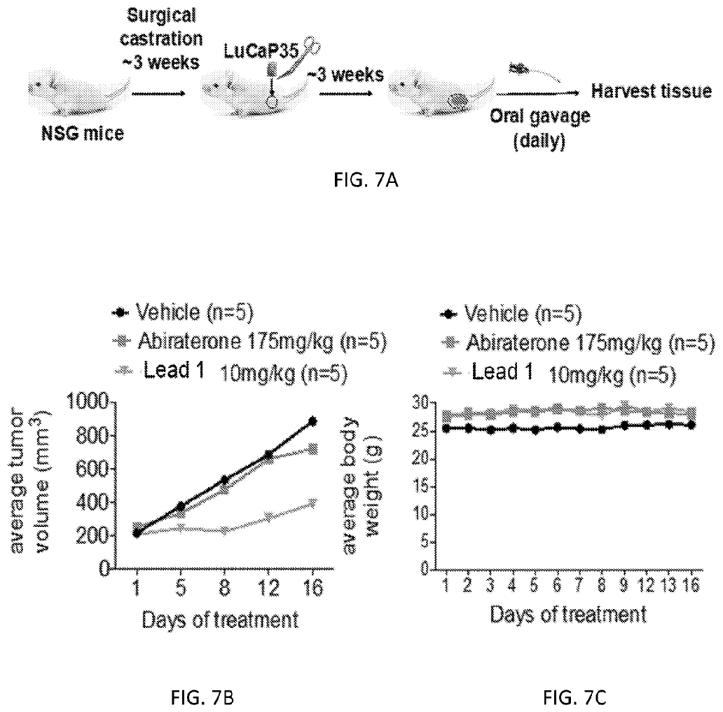

To determine whether the most potent and non-toxic synthetic lead 1 contributes to tumor growth, Chopra et al. performed LuCaP35CR patient derived xenograft (PDX) mice model studies (FIG. 7A). We treated castrate mice bearing LuCaP35CR xenografts with the potent lead 1 and the known CRPC drug abiraterone. Treatment with 10 mg/kg dose of lead 1 suppressed the tumor growth rapidly as compared to that of the non-treated vehicle mice. Abiraterone treatment did not show any significant effects on tumor growth even with the high dose of 175 mg/kg (FIG. 7B). Our results revealed that—lead 1 has better in vivo efficacy as compared to that of the current drug abiraterone in LuCaP35 CRPC model. Furthermore, treatment with lead 1 did not change the average body weight like the mice of both vehicle and abiraterone treatments over 16 days of period (FIG. 7C).

Experimental Methods

Reagents and solvents were purchased from commercial suppliers and used without further purification. NMR spectra were recorded on 500 MHz spectrometer (Bruker Ultrasheild Plus-500) at room temperature. Splitting patterns of the NMR peaks were noted as “s, d, t, q, and m” indicating “singlet, doublet, triplet, quartet, and multiplet” respectively. Chemical shifts (δ) are reported with MeOD (δ=3.30 ppm) or CDCl3 (δ=7.26 ppm) as internal standard. LC-MS spectra were recorded in Agilent Technologies 6460 Triple Quad LC/MS.

Cell Culture

The LNCaP, C4-2 & RWPE-1 cell lines were provided by Professor Timothy Ratliff (Purdue University Center for Cancer Research, USA). All these cells were and maintained at 37° C. with 5% CO2 atmosphere in a humidified incubator following American Type Culture Collection (ATCC) protocol. LNCaP cells were grown in RPMI-1640 (Gibco) supplemented with 10% FBS (Atlanta Biologics), 20 mM HEPES and 1% penicillin/streptomycin (Invitrogen). C4-2 cells were grown in 4:1 DMEM/F12-K medium (Gibco) supplemented with 10% FBS (Atlanta Biologics), 1% penicillin/streptomycin (Invitrogen), 3 mg/mL sodium bicarbonate, 5 μg/mL insulin, 1.36 ng/mL triiodothyronine, 5 μg/mL transferrin, 0.25 μg/mL biotin and 25 μg/mL adenine. For normal growth, RWPE-1 cells were maintained in Keratinocyte Serum Free Medium (K-SFM) (Invitrogen) supplemented with 0.05 mg/mL bovine pituitary extract (BPE) and 5 ng/mL epidermal growth factor (EGF). All the compounds were dissolved in dimethyl sulfoxide (DMSO) at high concentration (20 mM) followed by filtrations through a 0.22 μm syringe filter to make the stock solution which was further diluted with the culture medium to prepare the effective treatment concentration of the compounds. For experiments, cells were used from 3 to 12 passages from thawing.

Cell Proliferation Assay

The cell proliferation experiment was carried out by the ‘Cell Titer-Blue Cell Viability Assay,’ In this assay, approximately 5,000 cells/well were seeded in 100 μL growth media in poly-L-lysine coated 96-well plates. Next day, the cells were treated with additional 100 μL of various concentrations of the test compounds or DMSO-growth media as untreated control for 6 days in a humidified incubator at 37° C. and 5% CO2 atmosphere. 10 μL ‘Cell Titer-Blue Reagent’ was added directly to each well after 6 days and the plates were incubated for 3 h at 37° C. to allow cells to convert resazurin to resorufin, and the fluorescent signal was measured at 590 nm using a multiplate ELISA reader (Bio-Tek Synergy HT plate reader, Bio-Tek, Winooski, Vt.). The percentage of viable cells in a compound-treated sample was calculated by considering the absorbance of the DMSO-growth media treated sample as 100%. Data was analysed and IC50 value was calculated using GraphPad Prism Software. All experimental points were done in triplicate and experiments were repeated at least thrice.

Cell Viability Assay

The cell viability assay was performed using a standard methyl thiazolyldiphenyl tetrazolium bromide (MTT) assay. In this assay, approximately 5,000 cells/well were seeded in 100 μL growth media in poly-L-lysine coated 96-well plates. After 1 day of plating, the cells were treated with additional 100 μL of various concentrations of the test compounds or DMSO-growth media as untreated control for 3 days more in a humidified incubator at 37° C. and 5% CO2 atmosphere. After 3 days, the culture medium was replaced with 100 μL of MTT solution (using 1 mg/ml stock in growth media) per well and further incubated at 37° C. for 3-4 h. To dissolve the formazan crystals formed in each well by mitochondrial reductase from live cells, 100 μL DMSO was added in each well and shaked for 30 min at RT using an orbital shaker. The absorbance of each well was measured at 570 nm using a multiplate ELISA reader (Bio-Tek Synergy HT plate reader, Bio-Tek, Winooski, Vt.). The percentage of viable cells in a compound-treated sample was calculated by considering the absorbance of the DMSO-growth media treated sample as 100%. All experimental points were done in triplicate and experiments were repeated at least thrice.

Liver Microsome Assay

All the test compounds were incubated in duplicate at 3 μM concentration with mouse and human liver microsomes at 37° C. The reaction mixture contained microsomal enzyme in 100 mM potassium phosphate buffer of pH 7.4. Warfarin and verapamil were used as negative and positive control in this assay. After 0 min and 60 min incubation, aliquots were removed from each experimental and control reaction and mixed with an equal volume of ice-cold Stop Solution (0.3% acetic acid in acetonitrile). The samples were centrifuged to remove precipitated protein, and the supernatants were analyzed by LC-MS/MS to quantify the remaining parent molecule. Data represents % remaining as compared to the time zero concentration as 100%.

Western Blot

First, 0.5×106 cells/well were seeded in 6-well plate coated with 0.01% poly-L-lysine. Next day, cells were treated with 1 μM concentrations of drugs/compounds or 0.01% DMSO-growth media as vehicle control and the treatment was continued for 24 h in a humidified incubator at 37° C. and 5% CO2 atmosphere. Following treatment, cells were washed with cold PBS and lysed in TBSN buffer (20 mmol/L Tris, pH 8.0, 150 mmol/L NaCl, 1.5 mmol/L EDTA, 5 mmol/L EGTA, 0.5% Nonidet P-40, and 0.5 mmol/L Na3VO4) supplemented with 1× protease inhibitor cocktail tablets to obtain cellular lysates. The protein concentration of the whole cell lysate was quantified by Pierce BCA Protein Assay (Thermo Scientific). The lysate was run on 10% SDS-PAGE gel (40 μg of protein per lane) and β-actin (clone 8H10D10, Cell Signaling Technology) was used as a loading control. Proteins were transferred onto PVDF membrane and the nonspecific binding was blocked using 5% milk in PBS-T (0.05% Tween-20 in PBS buffer) for 1 h. Membrane was then incubated with primary antibody (anti-AR clone D6F11, Cell Signalling Technology) overnight at 4° C., washed for 1 h in PBS-T solution followed by 1.5 h incubation with horseradish peroxidase-conjugated goat anti-rabbit (for AR) and goat anti-mouse (for β-actin) IgG secondary antibodies. Next, the membrane was washed with PBS-T for 1 h and the protein bands were developed using a chemiluminescent reagent (Thermo Scientific) and visualized in FluorChem R system. Protein expression was normalized to β-actin and densitometry was calculated using ImageJ Software.

Immunofluorescent Staining

Cells (0.25×106) were seeded in 24 well plate coated with 0.01% poly-L-lysine. Next day, cells were treated with 0.01% DMSO control as vehicle and various compounds (1 μM). After 24 h treatment, cells were fixed with 4% paraformaldehyde for 15 minutes, washed with 0.1% Triton-X PBS, and permeabilized with methanol for 2 minutes. Upon wash with 0.1% Triton-X PBS buffer, cells were blocked with 3% bovine serum albumin made in PBS for 60 minutes, and incubated with primary antibodies (anti-AR clone D6F11, Cell Signalling Technology; anti-PR clone D8Q2J, Cell Signalling Technology; anti-SHBG, R & D Systems) for 12 hours at 4° C., followed by incubation with secondary antibodies (Alexafluor 594 goat anti-rabbit IgG for-AR and PR; Alexafluor 488 donkey anti-goat for SHBG) for 1 hour. Finally, cells were stained with 4,6-diamidino-2-phenylindole (DAPI) for 10 minutes, washed with PBS for three times and images were recorded using 40× object in in Nikon A1R-MP confocal laser microscope.

Synthesis of Compound 1:

Tibolone (62.4 mg, 0.2 mmol) was taken in a RB containing 10 ml of 9:1 THF/water mixture. Next, p-toluene sulfonic acid (38.0 mg, 0.2 mmol) was added to it and the mixture was refluxed for 48 h and the progress of the reaction was monitored by TLC. The reaction mixture was then evaporated to dryness to get the crude product. The crude product thus obtained was purified by column chromatography using 20% ethyl acetate in petroleum ether solvent mixture as eluent to give rise white solid pure compound 1 (Yield=70%). ¹H-NMR (500 MHz, CD₃OD, 25° C.): δ=5.80 (s, 1H), 2.88 (s, 1H), 2.57-2.54 (m, 1H), 2.38-2.29 (m, 4H), 2.27-2.22 (m, 1H), 2.21-2.15 (m, 1H), 2.00-1.93 (m, 3H), 1.76-1.71 (m, 2H), 1.69-1.61 (m, 3H), 1.58-1.50 (m, 1H), 1.40-1.22 (m, 3H), 1.17-1.12 (m, 1H), 1.10-0.90 (m, 3H), 0.79-0.78 (m, 3H) ppm. ¹³C-NMR (500 MHz, CD₃OD, 25° C.): δ=201.08, 167.85, 125.31, 87.27, 78.82, 73.43, 47.26, 47.09, 46.66, 45.81, 43.03, 42.75, 42.17, 38.27, 36.01, 32.24, 30.65, 26.41, 21.70, 11.76. LC-MS m/z (100%): Calculated for [(C21H2802)][M+H]⁺ 313.45; found, 313.60. HP-LC: retention time 9.874 minute.

Synthesis of Compound 2: Compound 2 was commercially available as Ethisterone. LC-MS m/z (100%): Calculated for [(C21H2802)][M+H]+ 313.44; found 313.20.

Synthesis of Compound 3:

To a vigorously stirred suspension of hydroxylammonium hydrochloride (13.90 mg, 0.2 mM), sodium acetate (24.60 mg, 0.3 mM) and 70% aqueous acetic acid (10 mL); compound 2 (62.48 mg, 0.2 mM) was added and stirring was continued for 72 h. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound 3 (E, Z) (Yield=55%). ¹H-NMR (500 MHz, CDCl₃, 25° C.): δ=6.56 (s, 1H), 5.85 (s, 1H), 5.81 (s, 1H), 5.35 (s, 1H), 3.12 (s, 1H), 3.10 (s, 1H), 2.58-2.57 (m, 2H), 2.43-2.40 (m, 8H), 2.32-2.29 (m, 2H), 2.28-2.21 (m, 2H), 2.18-2.16 (m, 6H), 2.04-2.0 (m, 6H), 1.98-1.91 (m, 6H), 1.89-1.69 (m, 2H), 1.67-1.62 (m, 6H), 1.60-1.18 (m, 6H), 0.91-0.87 (m, 6H), 0.78-0.76 (m, 6H) ppm. ¹³C-NMR (500 MHz, CDCl₃): δ=165.09, 150.46, 149.08, 126.52, 119.46, 87.40, 79.77, 74.13, 46.95, 46.03, 4.94, 43.71, 43.45, 43.35, 43.07, 42.98, 42.04, 41.77, 38.73, 32.33, 30.70, 30.29, 29.70, 26.84, 26.72, 26.64, 25.84, 22.23, 12.84, 12.79, 12.62. LC-MS m/z (100%): Calculated for [(C₂₁H₂₉NO₂)][M+H]⁺ 328.46; found, 328.30. HP-LC: retention times 9.913 and 10.121 minutes for E/Z isomers.

Synthesis of Compound 4:

To a vigorously stirred suspension of hydroxylammonium hydrochloride (13.90 mg, 0.2 mM), sodium acetate (24.60 mg, 0.3 mM) and 70% aqueous acetic acid (10 mL); norethisterone (59.70 mg, 0.2 mM) was added and stirring was continued for 72 h. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound 4 (E, Z) (Yield=52%). ¹H-NMR (500 MHz, CDCl₃, 25° C.): δ=6.62 (s, 1H), 5.87 (s, 1H), 5.82 (s, 1H), 5.30 (s, 1H), 3.16 (s, 1H), 3.13 (s, 1H), 2.58-2.57 (m, 2H), 2.43-2.40 (m, 8H), 2.32-2.29 (m, 2H), 2.28-2.21 (m, 2H), 2.18-2.16 (m, 6H), 2.04-2.0 (m, 6H), 1.98-1.91 (m, 6H), 1.89-1.69 (m, 2H), 1.67-1.62 (m, 6H), 1.60-1.20 (m, 6H), 0.89-0.85 (m, 6H) ppm. ¹³C-NMR (500 MHz, CDCl₃): δ=166.71, 157.31, 155.62, 151.45, 117.81, 111.30, 87.46, 79.74, 74.06, 49.37, 49.26, 49.18, 49.10, 46.88, 43.11, 42.54, 41.96, 41.23, 41.15, 41.01, 38.81, 36.48, 35.71, 35.47, 35.09, 32.46, 30.99, 30.80, 3065, 29.60, 27.19, 27.07, 2655, 26.29, 26.20, 26.11, 25.75, 22.99, 2099, 12.69. LC-MS m/z (100%): Calculated for [(C₂₀H₂₇NO₂)][M+H]⁺ 314.44; found, 314.30. HP-LC: retention times 9.530 and 9.775 minutes for E/Z isomers.

Synthesis of Compound 5:

To a solution of 2 (31.2 mg, 0.1 mM) in tBuOH (10 mL), excess chloranil was added and the reaction mixture was refluxed for 24 h. At the end, the solvent was evaporated to dryness to get the crude product, which was purified by Flash Chromatography to give the title compound (Yield=40%). LC-MS m/z: Calculated for [(C21H2702)][M+H]+ 311.20; found, 311.20.

Synthesis of Compound 6:

To a solution of norethisterone (29.8 mg, 0.1 mM) in ^{t}BuOH (10 mL), excess chloranil was added and the reaction mixture was refluxed for 24 h. The solvent was evaporated to get the crude product, which was purified by Flash Chromatography to give the title compound (Yield=45%). LC-MS m/z (100%): Calculated for [(C20H2502)][M+H]+ 297.19; found, 297.20.

Synthesis of Compound 7:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and ethisterone (0.2 mmol) in toluene (2 mL) was added and this mixture was stirred for another 10 minutes at RT followed by addition of 3-bromo-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 8:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-chloro-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 9:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and ethisterone (0.2 mmol) in toluene (2 mL) was added and this mixture was stirred for another 10 minutes at RT followed by addition of 2-chloro-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 10:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 3-bromo-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 11:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 3-bromo-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 12:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-chloro-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 13:

Tibolone (0.2 mmol) was taken in an RB flask containing 10 ml of THF. Next, p-toluene sulfonic acid (0.2 mmol) was added to it and the mixture was refluxed for 48 h and the progress of the reaction was monitored by TLC. The reaction mixture was then evaporated to dryness to get the crude product, which was purified by column chromatography using 10% ethyl acetate in petroleum ether solvent mixture as eluent to give rise the title compound.

Synthesis of Compound 14:

Norethisterone (0.2 mmol) was taken in an RB flask containing 10 ml of THF.

Next, p-toluene sulfonic acid (0.2 mmol) was added to it and the mixture was refluxed for 48 h and the progress of the reaction was monitored by TLC. The reaction mixture was then evaporated to dryness to get the crude product, which was purified by column chromatography using 10% ethyl acetate in petroleum ether solvent mixture as eluent to give rise the title compound.

Synthesis of Compound 15:

Compound 1 (0.2 mM) and sodium hydride (0.3 mM) were dissolved in 10 ml dry THF and the mixture was stirred for 60 minutes at RT followed by addition of excess methyl iodide. The reaction was continued for 48 h at RT. After the reaction is complete, the excess sodium hydride was decomposed by dropwise addition of water. The product formed was separated from the reaction mixture by partial evaporation of the solvent followed by extraction. The obtained crude product was purified by Flash Chromatography to give the title compound.

Synthesis of Compound 16:

Norethisterone (0.2 mM) and sodium hydride (0.3 mM) were dissolved in 10 ml dry THF and the mixture was stirred for 60 minutes at RT followed by addition of excess methyl iodide. The reaction was continued for 48 h at RT. After the reaction is complete, the excess sodium hydride was decomposed by dropwise addition of water. The product formed was separated from the reaction mixture by partial evaporation of the solvent followed by extraction. The obtained crude product was purified by Flash Chromatography to give the title compound.

Synthesis of Compound 17:

Compound 1 (0.2 mM) and sodium hydride (0.3 mM) were dissolved in 10 ml dry THF and the mixture was stirred for 60 minutes at RT followed by addition of excess ethyl iodide. The reaction was continued for 48 h at RT. After the reaction is complete, the excess sodium hydride was decomposed by dropwise addition of water. The product formed was separated from the reaction mixture by partial evaporation of the solvent followed by extraction. The obtained crude product was purified by Flash Chromatography to give the title compound.

Synthesis of Compound 18:

Norethisterone (0.2 mM) and sodium hydride (0.3 mM) were dissolved in 10 ml dry THF and the mixture was stirred for 60 minutes at RT followed by addition of excess ethyl iodide. The reaction was continued for 48 h at RT. After the reaction is complete, the excess sodium hydride was decomposed by dropwise addition of water. The product formed was separated from the reaction mixture by partial evaporation of the solvent followed by extraction. The obtained crude product was purified by Flash Chromatography to give the title compound.

Synthesis of Compound 19:

To a vigorously stirred suspension of methyl amine (0.2 mM), sodium acetate (0.3 mM), 70% aqueous acetic acid (10 mL) and compound 1 (0.2 mM) was added and stirring was continued for 72 h at RT. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound.

Synthesis of Compound 20:

To a vigorously stirred suspension of methyl amine (0.2 mM), sodium acetate (0.3 mM), 70% aqueous acetic acid (10 mL) and norethisterone (0.2 mM) was added and stirring was continued for 72 h at RT. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound.

Synthesis of Compound 21:

To a vigorously stirred suspension of hydrazine hydrochloride (0.2 mM), sodium acetate (0.3 mM), 70% aqueous acetic acid (10 mL) and compound 1 (0.2 mM) was added and stirring was continued for 72 h at RT. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound.

Synthesis of Compound 22:

To a vigorously stirred suspension of hydrazine hydrochloride (0.2 mM), sodium acetate (0.3 mM), 70% aqueous acetic acid (10 mL) and norethisterone (0.2 mM) was added and stirring was continued for 72 h at RT. The reaction mixture was poured into 100 mL of cold water. The precipitated product was filtered off, washed successively with water, 5% aqueous ammonium hydroxide solution, and water until neutral, and then dried below 50° C. The obtained crude product was purified by Flash Chromatography to give the title compound.

Synthesis of Compound 23:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-chloro-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 24:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-chloro-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 25:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 3-bromo-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 26:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 3-bromo-pyridine (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 27:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-iodo-phenol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 28:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-iodo-phenol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 29:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-bromo-aniline (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 30:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-bromo-aniline (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 31:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-iodo-phenol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 32:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-iodo-phenol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 33:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 2-bromo-aniline (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 34:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 2-bromo-aniline (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 35:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and compound 1 (0.2 mmol) in toluene (2 mL) was added and this mixture was stirred for another 10 minutes at RT followed by addition of 4-chlorobenzyl alcohol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 36:

In a two neck RB flask equipped with magnetic stirring bar and reflux condenser, Pd(PPh₃)₂Cl₂ (0.04 mmol), CuI (0.04 mmol), and PPh₃ (catalytic amount) were added under argon atmosphere followed by dry degassed toluene (5 mL) and triethylamine (1.0 mL). The mixture was stirred for 10 min. and norethisterone (0.2 mmol) in toluene (2 mL) was added and this mixture was stirred for another 10 minutes at RT followed by addition of 4-chlorobenzyl alcohol (0.2 mmol). The reaction mixture was stirred at 70-80° C. temperature for 12 h. Reaction was quenched with 10% aqueous citric acid (20 mL) and extracted with DCM (3×30 mL). Combined extracts were washed with 10% aqueous NaOH (20 mL), water and dried with anhydrous magnesium sulfate. Solvent was removed by rotary evaporation and the crude product was purified using flash chromatography to get the respective pure compound.

Synthesis of Compound 37:

Palladium on carbon (Pd/C), 10% weight of the substrate was added to a solution of compound 1 (0.2 mmol) in MeOH (10 ml). The reaction mixtures were stirred under a slight pressure of hydrogen atmosphere (balloon) at room temperature for 6 h. The resulting mixtures were then filtered, and the filtrate was concentrated in vacuo to obtain the corresponding reduced product.

Synthesis of Compound 38:

Palladium on carbon (Pd/C), 10% weight of the substrate was added to a solution of norethisterone (0.2 mmol) in MeOH (10 ml). The reaction mixtures were stirred under a slight pressure of hydrogen atmosphere (balloon) at room temperature for 6 h. The resulting mixtures were then filtered, and the filtrate was concentrated in vacuo to obtain the corresponding reduced product.

Synthesis of Compound 39:

In this reaction, first compound 1 (0.2 mM) was dissolved in 20 ml ice-cold methanol and then the reducing agent sodium borohydride (0.2 mM) was added and stirring was continued for 12 h at the ice-cold condition. After the reaction is complete, the excess sodium borohydride was decomposed by acidifying the reaction mixture (slowly and while stirring) using aqueous HCl. The product formed was separated from the reaction mixture by partial evaporation of the solvent followed by extraction. The obtained crude product was purified by Flash Chromatography to give the title compound (a, 0).

Synthesis of Compound 40:

In this reaction, first norethisterone (0.2 mM) was dissolved in 20 ml ice-cold methanol and then the reducing agent sodium borohydride (0.2 mM) was added and stirring was continued for 12 h at the ice-cold condition. After the reaction is complete, the excess sodium borohydride was decomposed by acidifying the reaction mixture (slowly and while stirring) using aqueous HCl. The product formed was separated from the reaction mixture by partial evaporation of the solvent followed by extraction. The obtained crude product was purified by Flash Chromatography to give the title compound (α, β).

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope.

1. A compound having the formula: 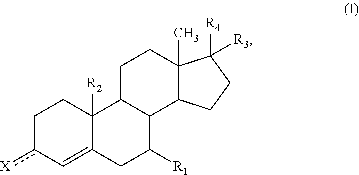
or a pharmaceutically acceptable salt thereof, wherein [symbol]
represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, aminoalkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is absent or hydrogen, hydroxyl, halo, azido, nitro, cyano, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

2. The compound according to claim 1, wherein the compound has the following formula: 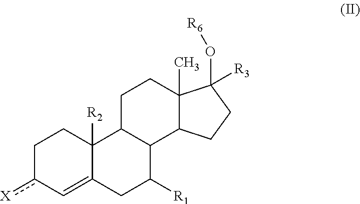
or a pharmaceutically acceptable salt thereof, wherein [symbol]
represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, aminoalkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

3. The compound according to claim 1, wherein the compound has the following formula: 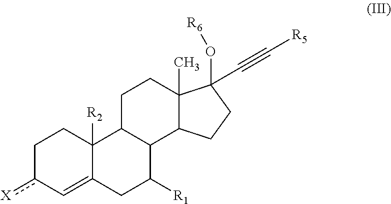
or a pharmaceutically acceptable salt thereof, wherein [symbol]
represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₅ is hydrogen, an alkyl, alkenyl, alkynyl, alkylalkynyl, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₆ is hydrogen, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

4. The compound according to claim 3, wherein
R₁ and R₂ are independently hydrogen or methyl;
and
R₆ is hydrogen or a C1-C6 alkyl.

5. The compound according to claim 4, wherein
R₅ and R₆ are hydrogen.

6. The compound according to claim 4, wherein said compound is a compound of compounds 1-6 of FIG. 2.

7. The compound according to claim 4, wherein said compound is a compound of compounds 15-22 of FIG. 2.

8. The compound according to claim 4, wherein said compound is a compound of compounds 23-40 of FIG. 2.

9. The compound according to claim 3, wherein [symbol] represents a double bond, and X is O, S, NH, N—OH, N—NH₂, or NR₇, wherein R₇ is an C1-C6 alkyl;
R₁ and R₂ are independently hydrogen or methyl; and
R₅ and R₆ are hydrogen.

10. The compound according to claim 3,
R₁ and R₂ are independently hydrogen or methyl;
R₅ is 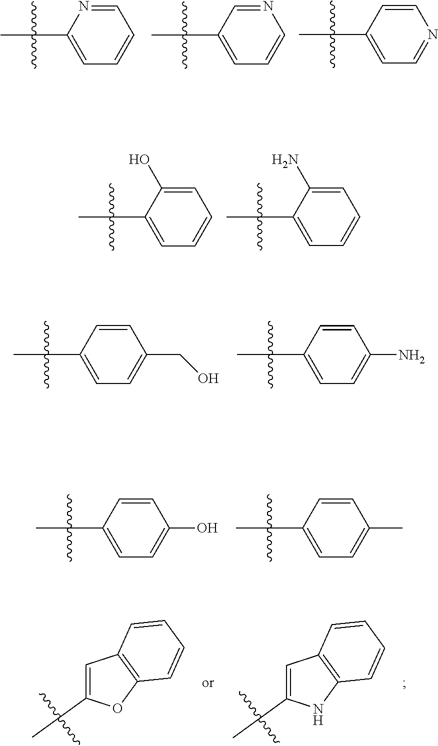
and
R₆ is hydrogen.

11. (canceled)

12. The compound of claim 1 having the formula: 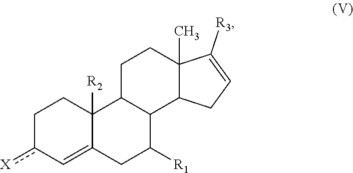
or a pharmaceutically acceptable salt thereof, wherein [symbol]
represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl; and
R₃ is hydrogen, hydroxyl, thiol, halo, azido, nitro, cyano, an alkyl, alkenyl, alkynyl, alkylalkynyl, alkyloxy, hydroxyalkyl, aminoalkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

13. The compound according to claim 12, wherein the compound comprises a compound of compounds 7-14 of FIG. 2, or

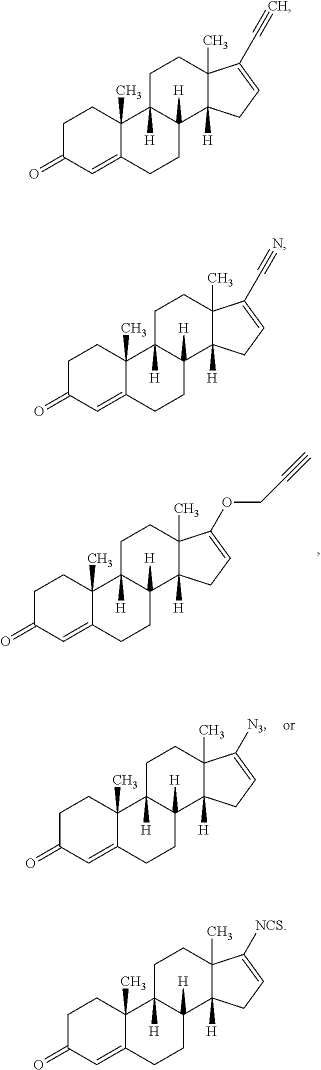

14. The compound according to claim 1, wherein the compound is

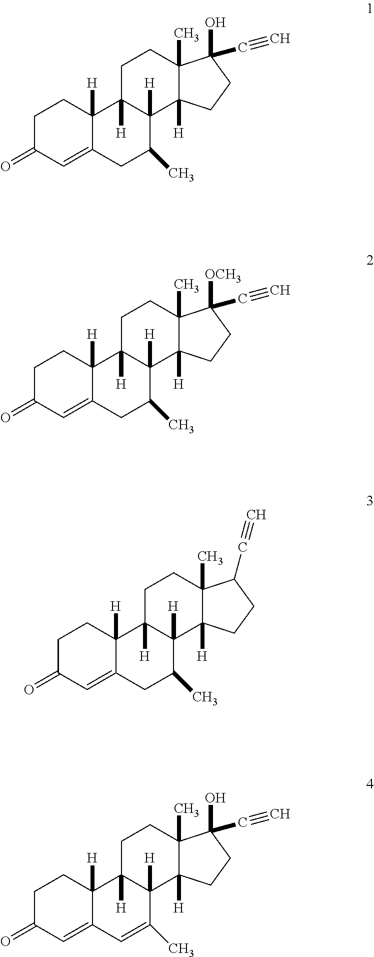

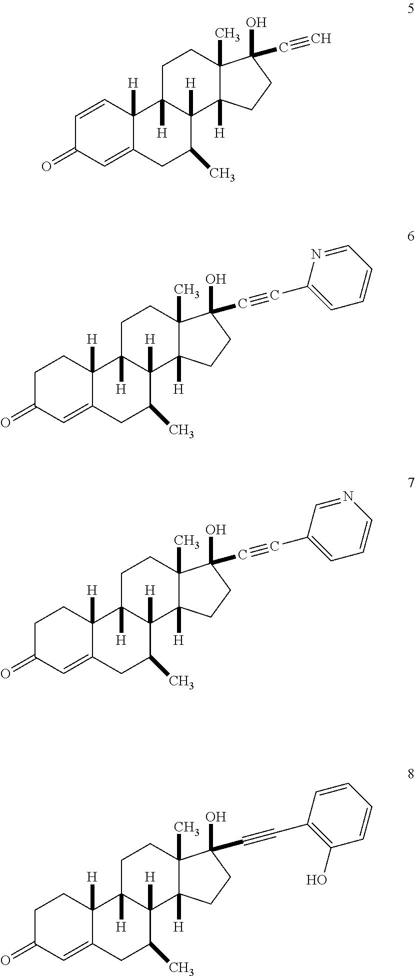

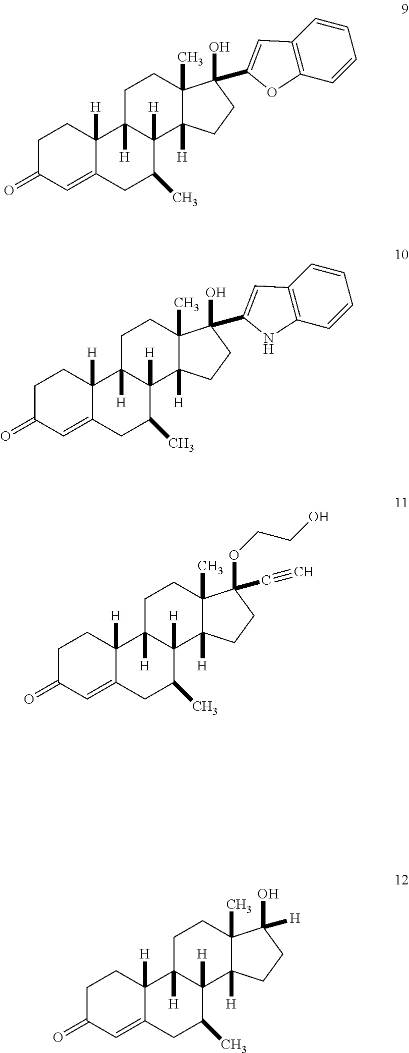

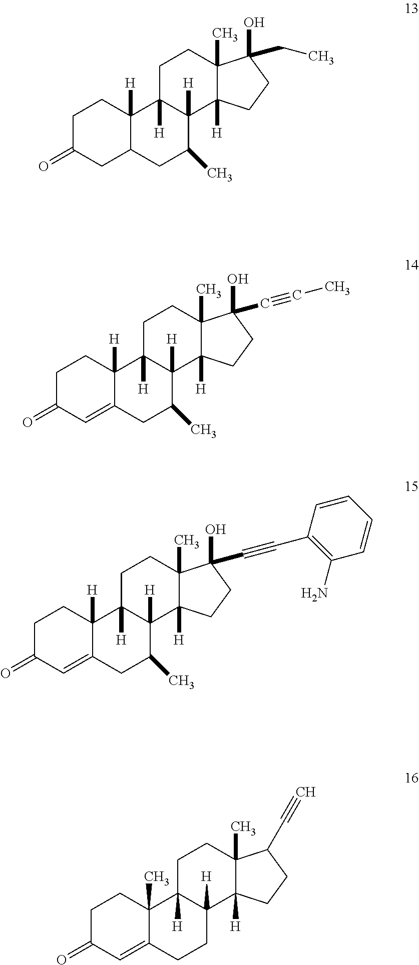

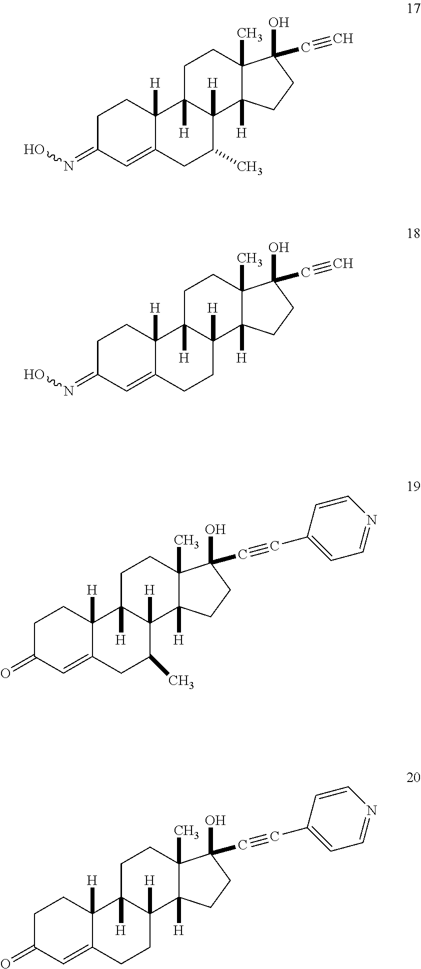

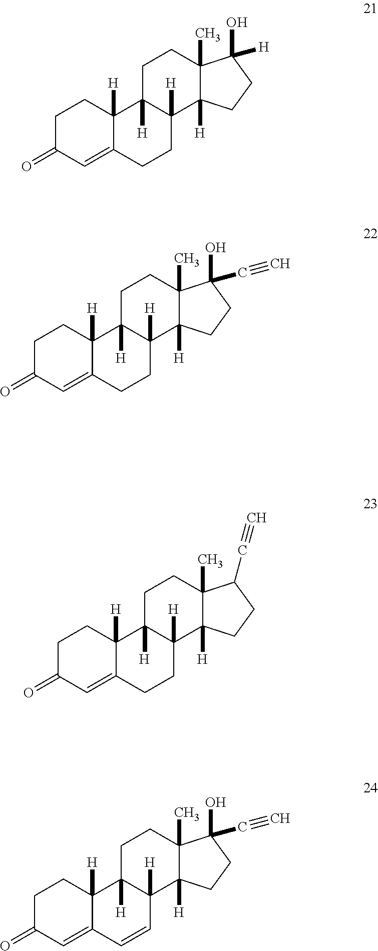

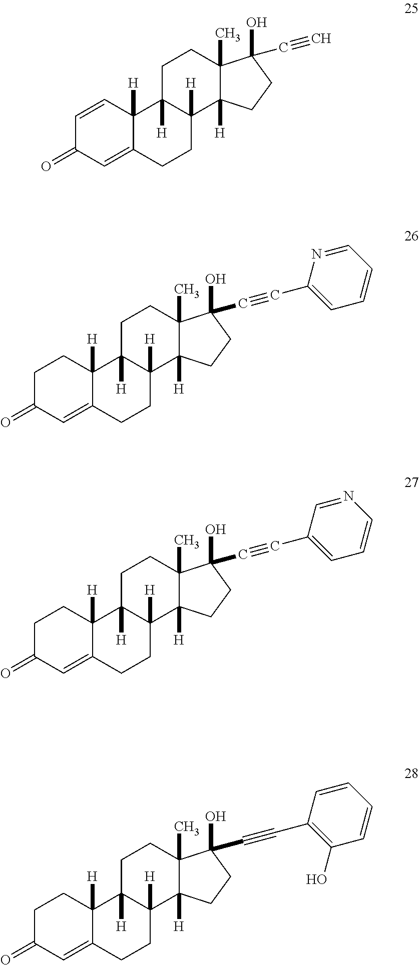

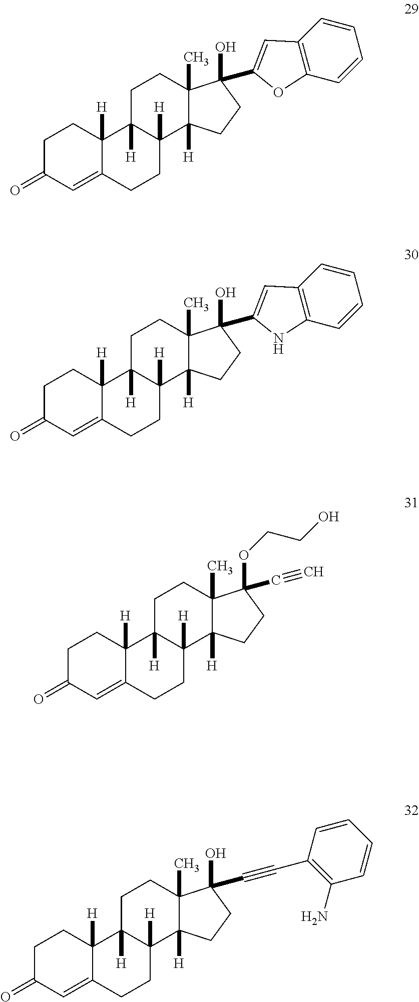

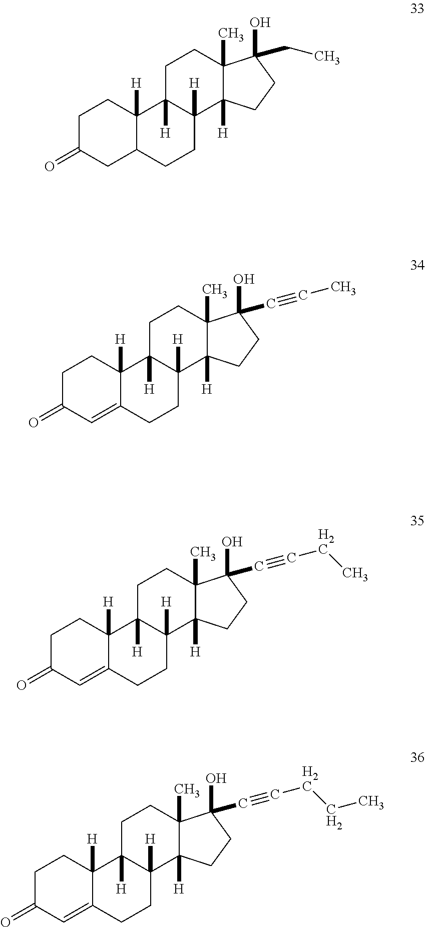

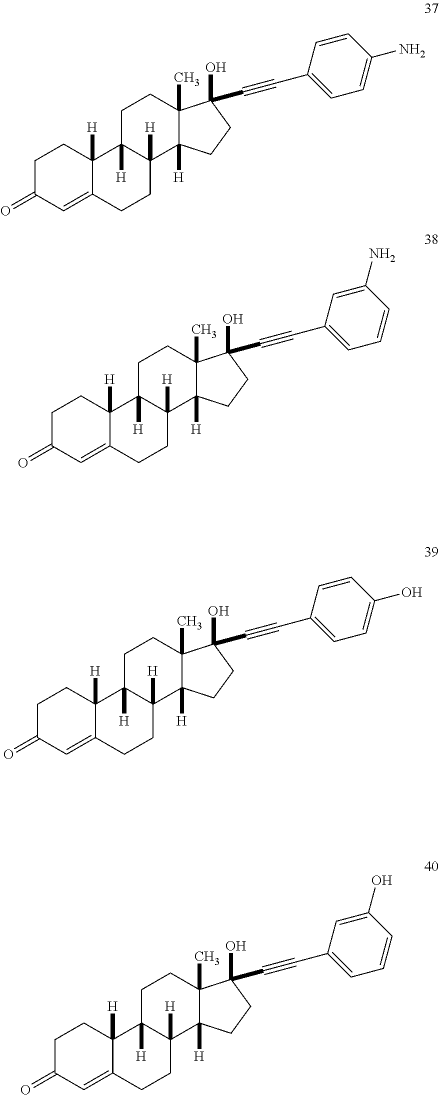

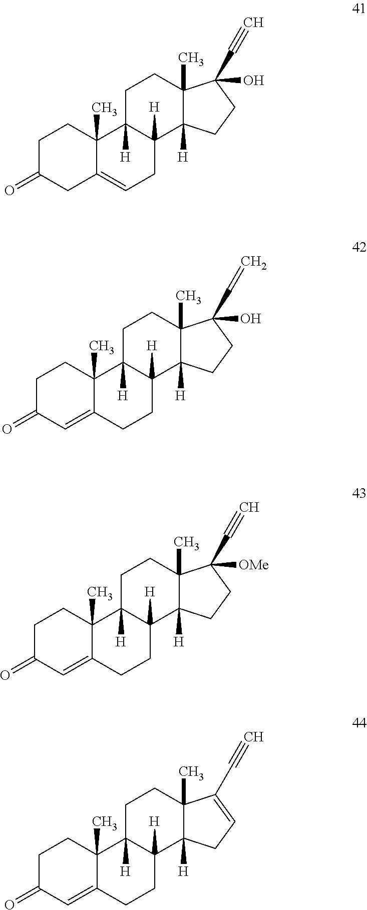

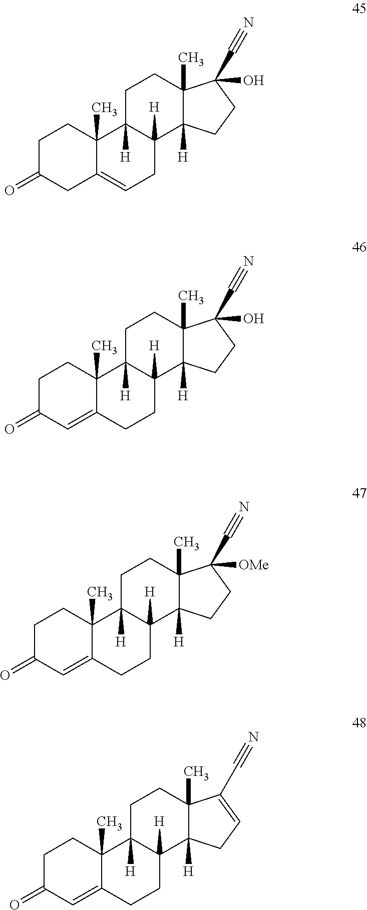

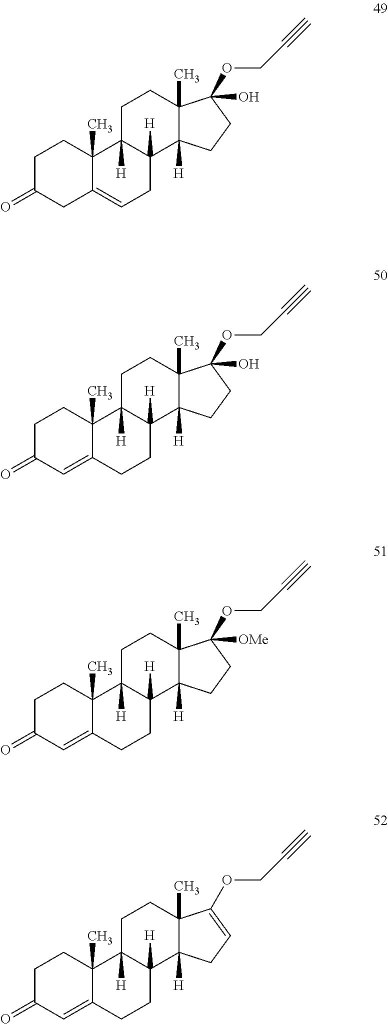

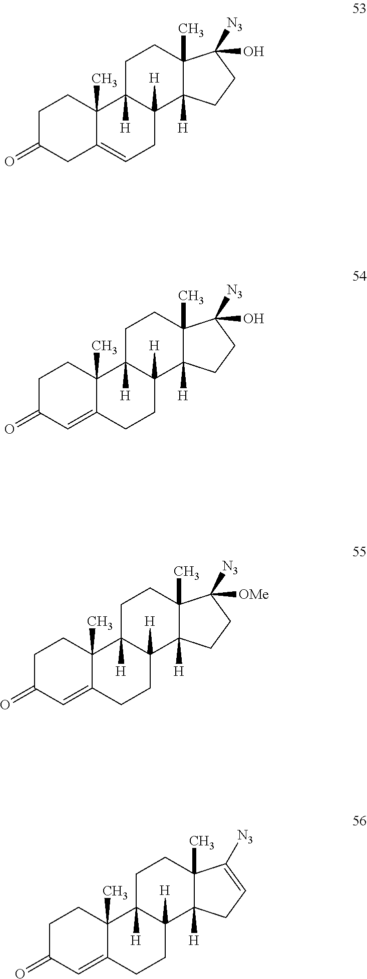

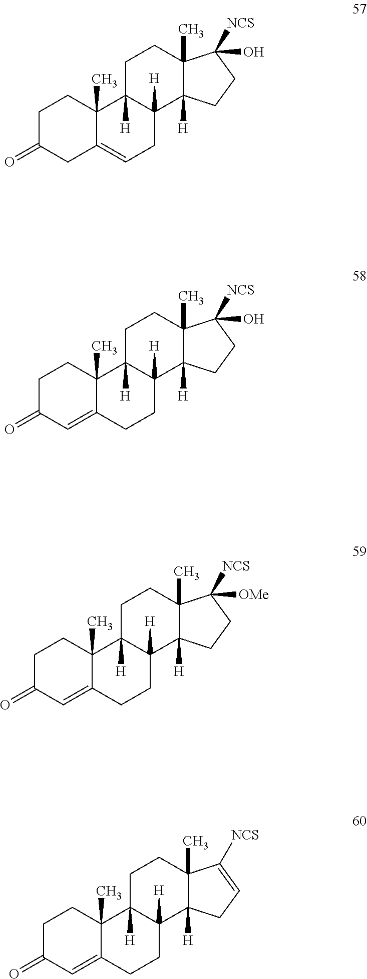

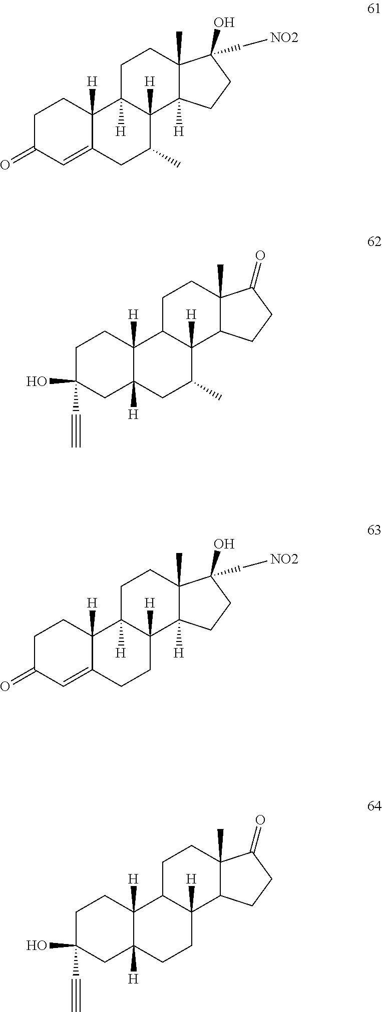

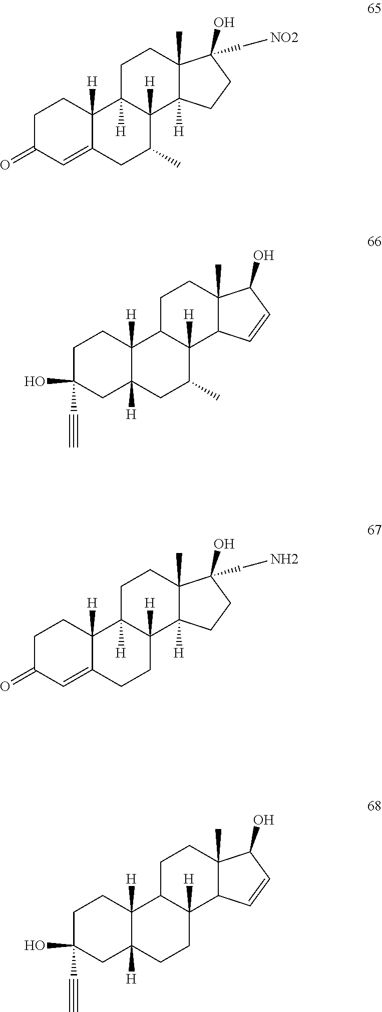

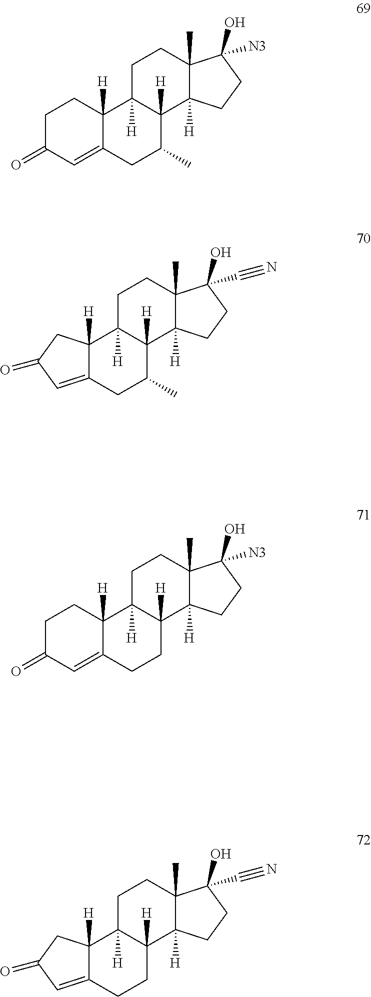

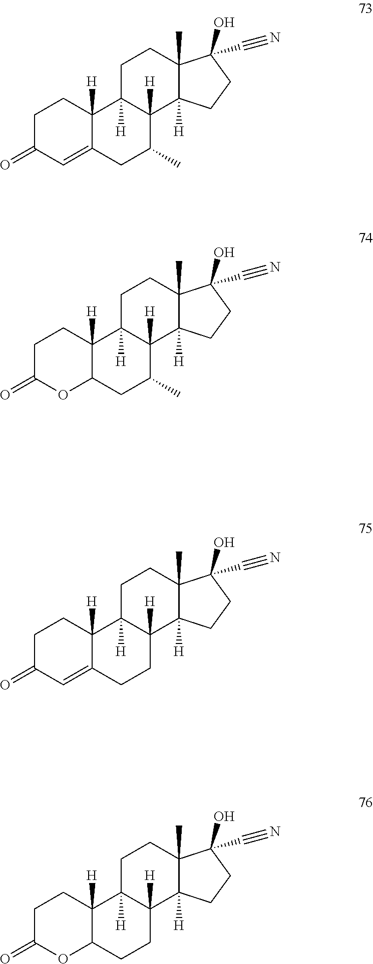

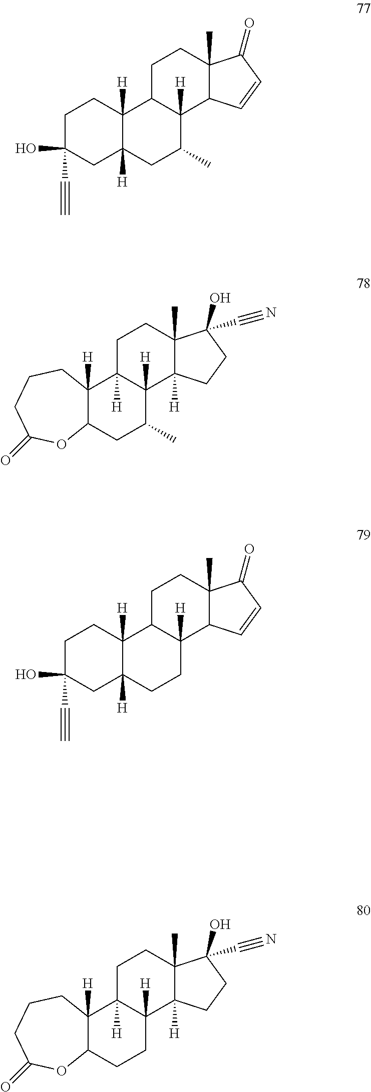

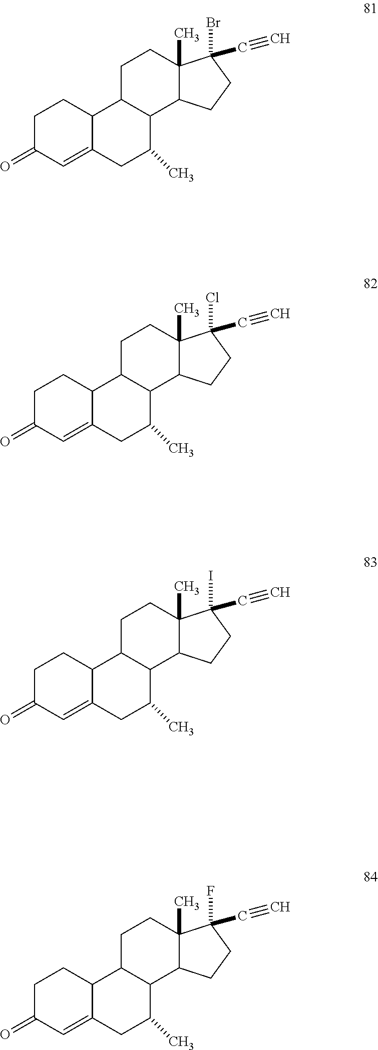 [SMILES: C#C[C@@]1(Br)CCC2C3C(CC[C@@]21C)C1CCC(=O)C=C1C[C@H]3C ; C#C[C@@]1(Cl)CCC2C3C(CC[C@@]21C)C1CCC(=O)C=C1C[C@H]3C ; C#C[C@@]1(I)CCC2C3C(CC[C@@]21C)C1CCC(=O)C=C1C[C@H]3C ; C#C[C@@]1(F)CCC2C3C(CC[C@@]21C)C1CCC(=O)C=C1C[C@H]3C]

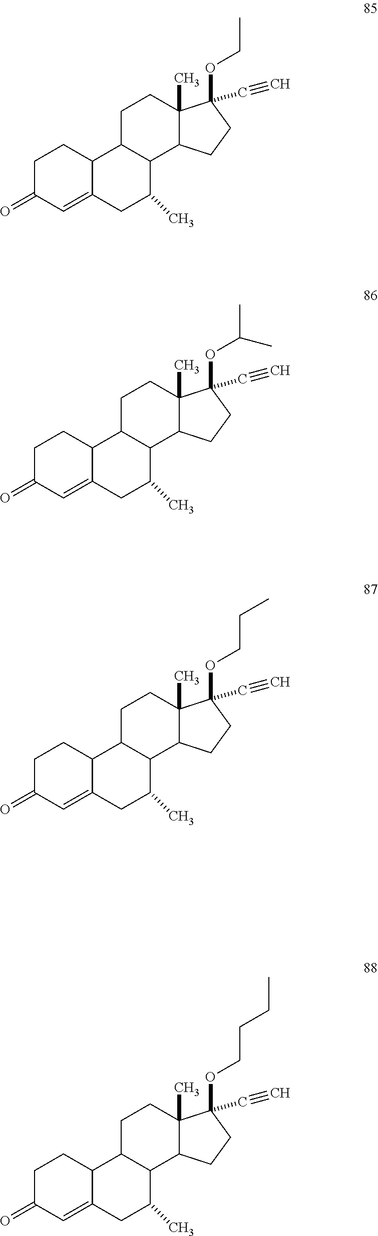 [SMILES: C#C[C@]1(OCC)CCC2C3C(CC[C@@]21C)C1CCC(=O)C=C1C[C@H]3C ; C#C[C@]1(OC(C)C)CCC2C3C(CC[C@@]21C)C1CCC(=O)C=C1C[C@H]3C ; C#C[C@]1(OCCC)CCC2C3C(CC[C@@]21C)C1CCC(=O)C=C1C[C@H]3C ; C#C[C@]1(OCCCC)CCC2C3C(CC[C@@]21C)C1CCC(=O)C=C1C[C@H]3C]

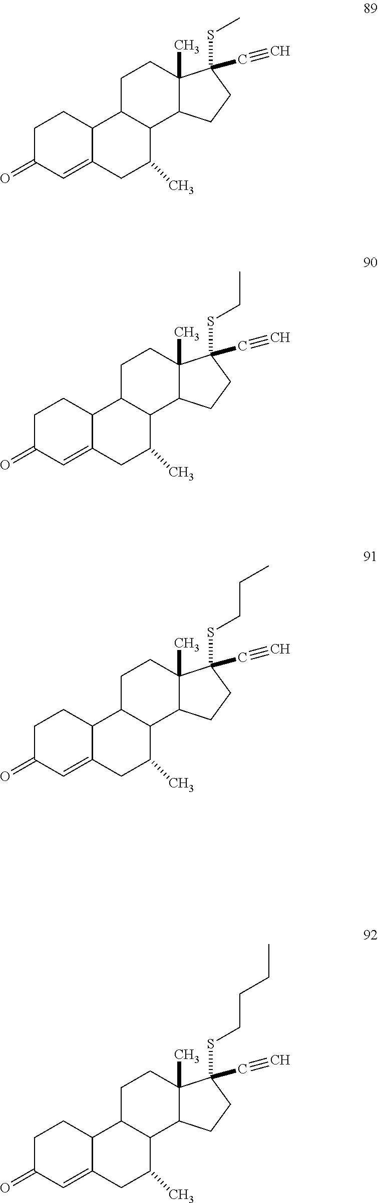 [SMILES: C#C[C@@]1(SC)CCC2C3C(CC[C@@]21C)C1CCC(=O)C=C1C[C@H]3C ; C#C[C@@]1(SCC)CCC2C3C(CC[C@@]21C)C1CCC(=O)C=C1C[C@H]3C ; C#C[C@@]1(SCCC)CCC2C3C(CC[C@@]21C)C1CCC(=O)C=C1C[C@H]3C ; C#C[C@@]1(SCCCC)CCC2C3C(CC[C@@]21C)C1CCC(=O)C=C1C[C@H]3C]

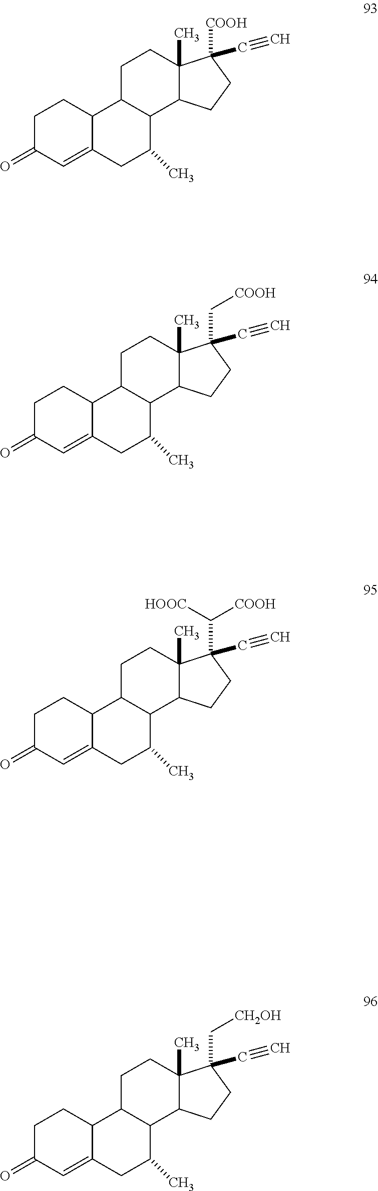 [SMILES: C#C[C@@]1(C(=O)O)CCC2C3C(CC[C@@]21C)C1CCC(=O)C=C1C[C@H]3C ; C#C[C@@]1(CC(=O)O)CCC2C3C(CC[C@@]21C)C1CCC(=O)C=C1C[C@H]3C ; C#C[C@@]1(C(C(=O)O)C(=O)O)CCC2C3C(CC[C@@]21C)C1CCC(=O)C=C1C[C@H]3C ; C#C[C@@]1(CCO)CCC2C3C(CC[C@@]21C)C1CCC(=O)C=C1C[C@H]3C]

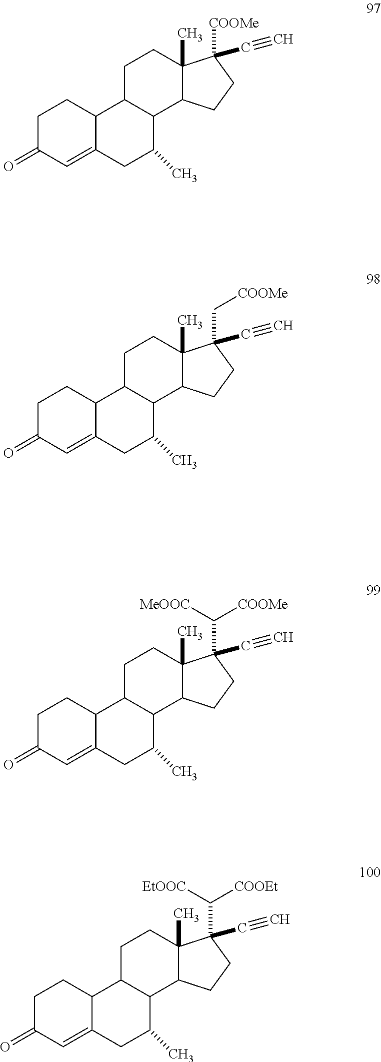 [SMILES: C#C[C@@]1(C(=O)OC)CCC2C3C(CC[C@@]21C)C1CCC(=O)C=C1C[C@H]3C ; C#C[C@@]1(CC(=O)OC)CCC2C3C(CC[C@@]21C)C1CCC(=O)C=C1C[C@H]3C ; C#C[C@@]1(C(C(=O)OC)C(=O)OC)CCC2C3C(CC[C@@]21C)C1CCC(=O)C=C1C[C@H]3C ; C#C[C@@]1(C(C(=O)OCC)C(=O)OCC)CCC2C3C(CC[C@@]21C)C1CCC(=O)C=C1C[C@H]3C]

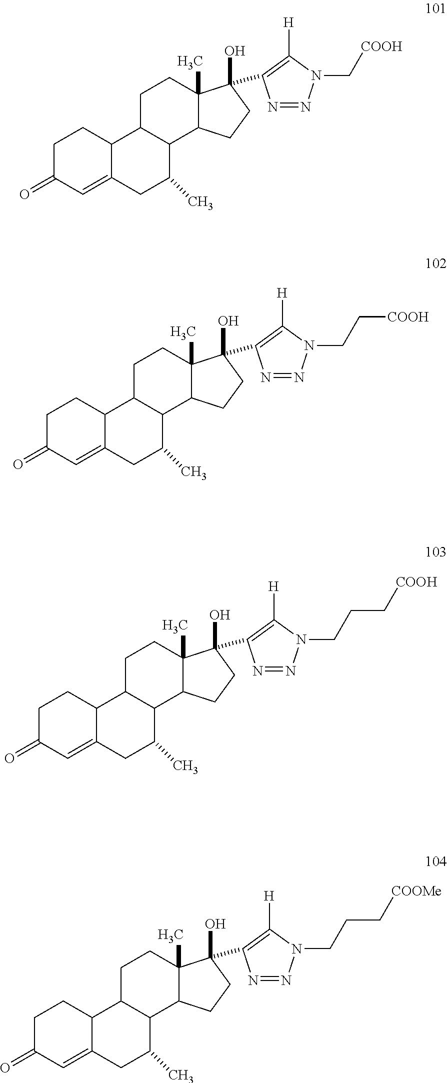 [SMILES: C[C@@H]1CC2=CC(=O)CCC2C2CC[C@@]3(C)C(CC[C@@]3(O)c3cn(CC(=O)O)nn3)C21 ; C[C@@H]1CC2=CC(=O)CCC2C2CC[C@@]3(C)C(CC[C@@]3(O)c3cn(CCC(=O)O)nn3)C21 ; C[C@@H]1CC2=CC(=O)CCC2C2CC[C@@]3(C)C(CC[C@@]3(O)c3cn(CCCC(=O)O)nn3)C21 ; COC(=O)CCCn1cc([C@]2(O)CCC3C4C(CC[C@@]32C)C2CCC(=O)C=C2C[C@H]4C)nn1]

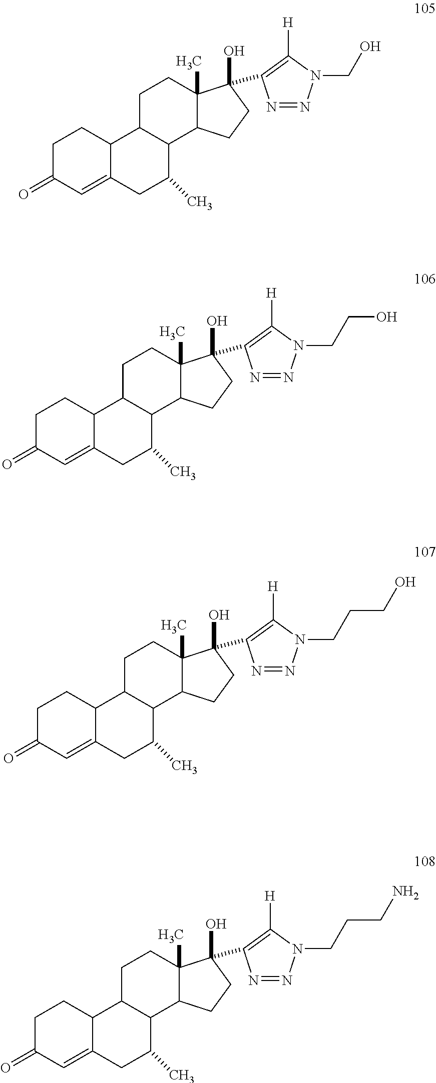 [SMILES: C[C@@H]1CC2=CC(=O)CCC2C2CC[C@@]3(C)C(CC[C@@]3(O)c3cn(CO)nn3)C21 ; C[C@@H]1CC2=CC(=O)CCC2C2CC[C@@]3(C)C(CC[C@@]3(O)c3cn(CCO)nn3)C21 ; C[C@@H]1CC2=CC(=O)CCC2C2CC[C@@]3(C)C(CC[C@@]3(O)c3cn(CCCO)nn3)C21 ; C[C@@H]1CC2=CC(=O)CCC2C2CC[C@@]3(C)C(CC[C@@]3(O)c3cn(CCCN)nn3)C21]

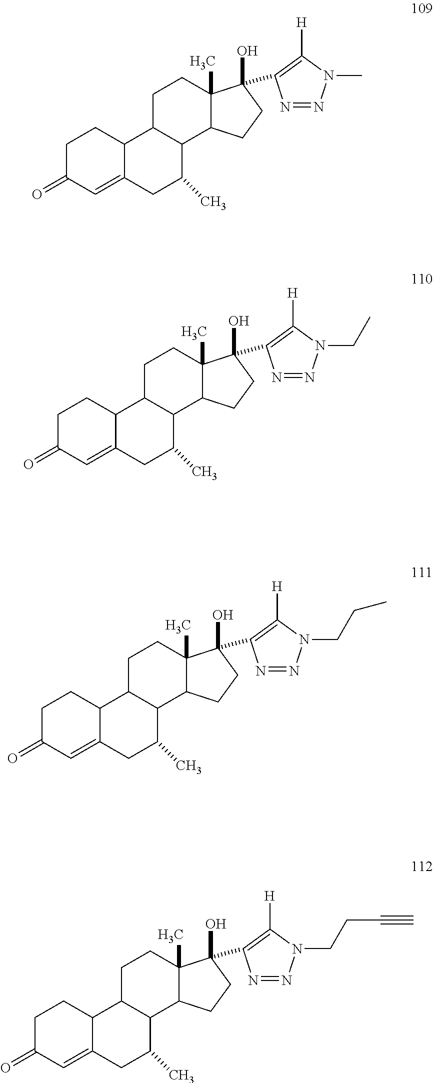 [SMILES: C[C@@H]1CC2=CC(=O)CCC2C2CC[C@@]3(C)C(CC[C@@]3(O)c3cn(C)nn3)C21 ; CCn1cc([C@]2(O)CCC3C4C(CC[C@@]32C)C2CCC(=O)C=C2C[C@H]4C)nn1 ; CCCn1cc([C@]2(O)CCC3C4C(CC[C@@]32C)C2CCC(=O)C=C2C[C@H]4C)nn1 ; C#CCCn1cc([C@]2(O)CCC3C4C(CC[C@@]32C)C2CCC(=O)C=C2C[C@H]4C)nn1]

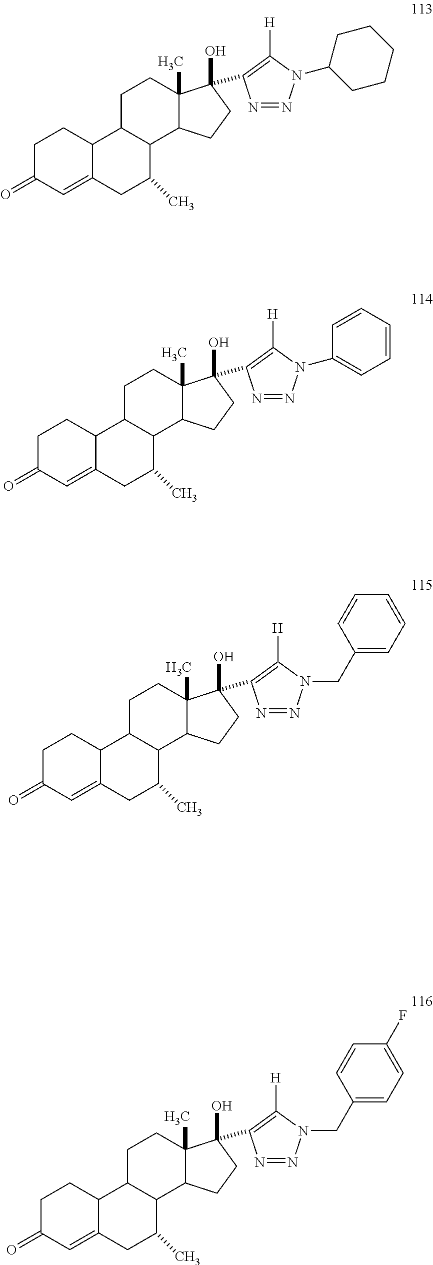 [SMILES: C[C@@H]1CC2=CC(=O)CCC2C2CC[C@@]3(C)C(CC[C@@]3(O)c3cn(C4CCCCC4)nn3)C21 ; C[C@@H]1CC2=CC(=O)CCC2C2CC[C@@]3(C)C(CC[C@@]3(O)c3cn(-c4ccccc4)nn3)C21 ; C[C@@H]1CC2=CC(=O)CCC2C2CC[C@@]3(C)C(CC[C@@]3(O)c3cn(Cc4ccccc4)nn3)C21 ; C[C@@H]1CC2=CC(=O)CCC2C2CC[C@@]3(C)C(CC[C@@]3(O)c3cn(Cc4ccc(F)cc4)nn3)C21]

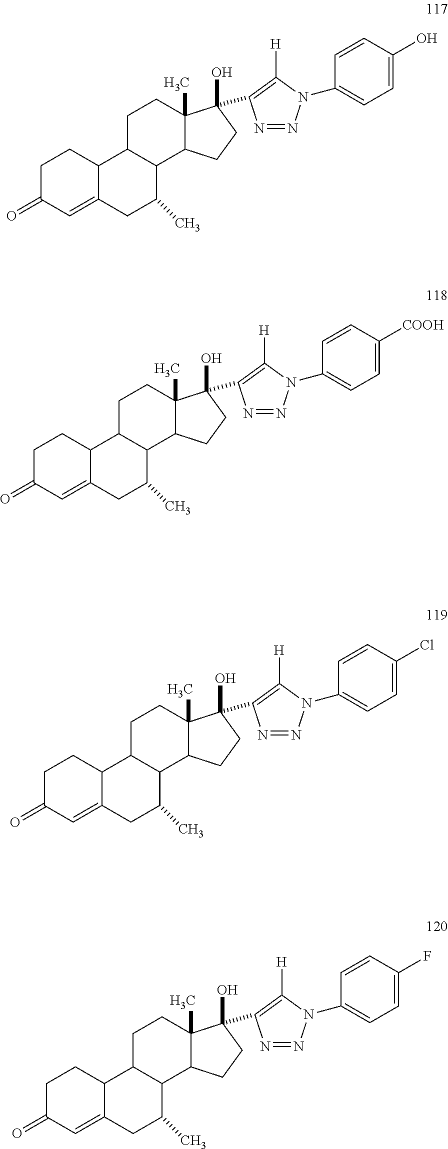 [SMILES: C[C@@H]1CC2=CC(=O)CCC2C2CC[C@@]3(C)C(CC[C@@]3(O)c3cn(-c4ccc(O)cc4)nn3)C21 ; C[C@@H]1CC2=CC(=O)CCC2C2CC[C@@]3(C)C(CC[C@@]3(O)c3cn(-c4ccc(C(=O)O)cc4)nn3)C21 ; C[C@@H]1CC2=CC(=O)CCC2C2CC[C@@]3(C)C(CC[C@@]3(O)c3cn(-c4ccc(Cl)cc4)nn3)C21 ; C[C@@H]1CC2=CC(=O)CCC2C2CC[C@@]3(C)C(CC[C@@]3(O)c3cn(-c4ccc(F)cc4)nn3)C21]

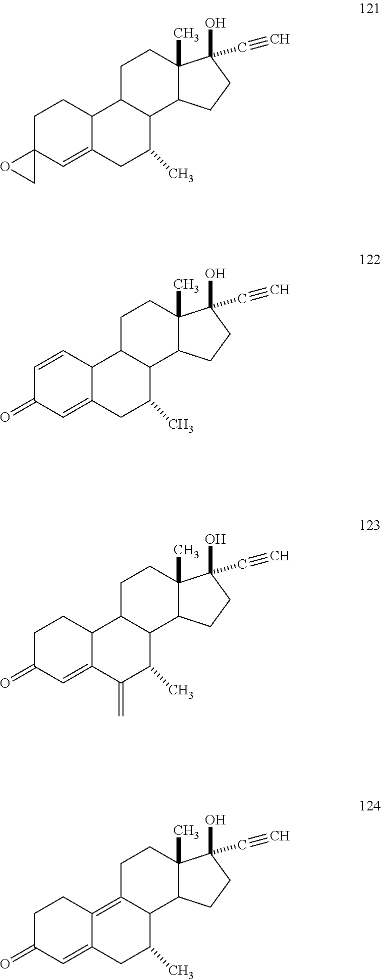 [SMILES: C#C[C@]1(O)CCC2C3C(CC[C@@]21C)C1CCC2(C=C1C[C@H]3C)CO2 ; C#C[C@]1(O)CCC2C3C(CC[C@@]21C)C1C=CC(=O)C=C1C[C@H]3C ; C#C[C@]1(O)CCC2C3C(CC[C@@]21C)C1CCC(=O)C=C1C(=C)[C@H]3C ; C#C[C@]1(O)CCC2C3C(=C4CCC(=O)C=C4C[C@H]3C)CC[C@@]21C]

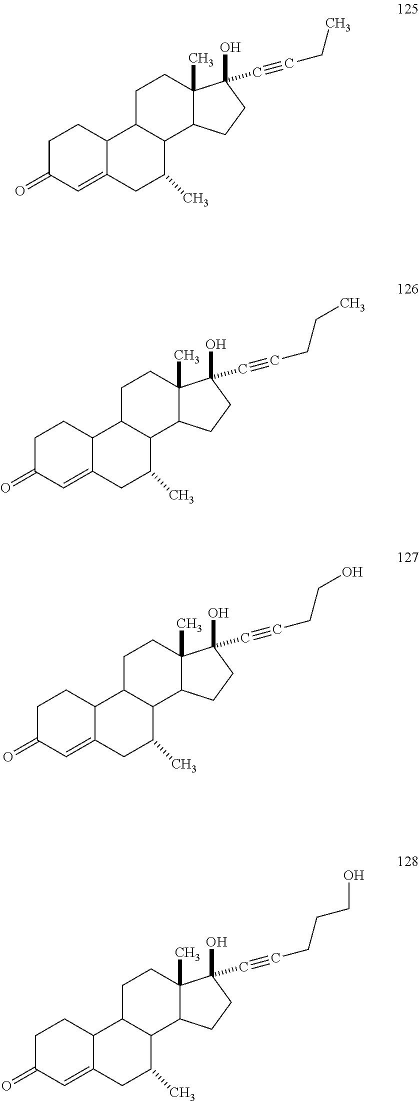 [SMILES: CCC#C[C@]1(O)CCC2C3C(CC[C@@]21C)C1CCC(=O)C=C1C[C@H]3C ; CCCC#C[C@]1(O)CCC2C3C(CC[C@@]21C)C1CCC(=O)C=C1C[C@H]3C ; C[C@@H]1CC2=CC(=O)CCC2C2CC[C@@]3(C)C(CC[C@@]3(O)C#CCCO)C21 ; C[C@@H]1CC2=CC(=O)CCC2C2CC[C@@]3(C)C(CC[C@@]3(O)C#CCCCO)C21]

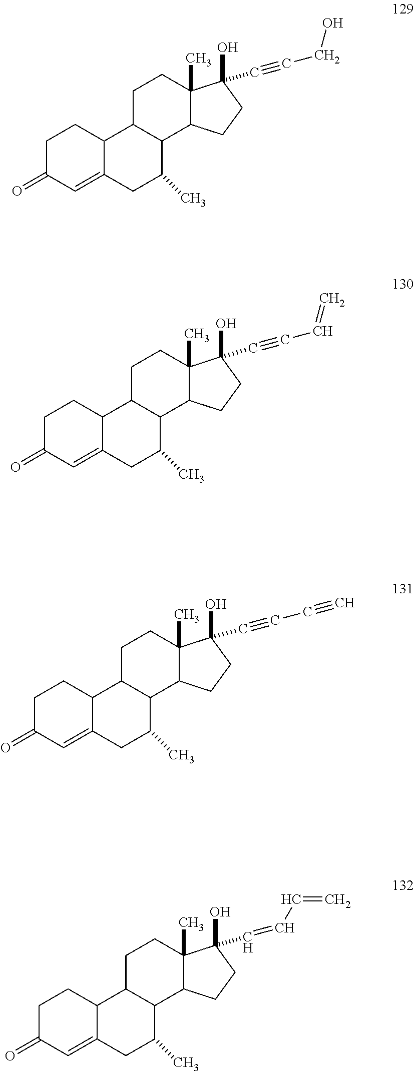 [SMILES: C[C@@H]1CC2=CC(=O)CCC2C2CC[C@@]3(C)C(CC[C@@]3(O)C#CCO)C21 ; C=CC#C[C@]1(O)CCC2C3C(CC[C@@]21C)C1CCC(=O)C=C1C[C@H]3C ; C#CC#C[C@]1(O)CCC2C3C(CC[C@@]21C)C1CCC(=O)C=C1C[C@H]3C ; C=CC=C[C@]1(O)CCC2C3C(CC[C@@]21C)C1CCC(=O)C=C1C[C@H]3C]

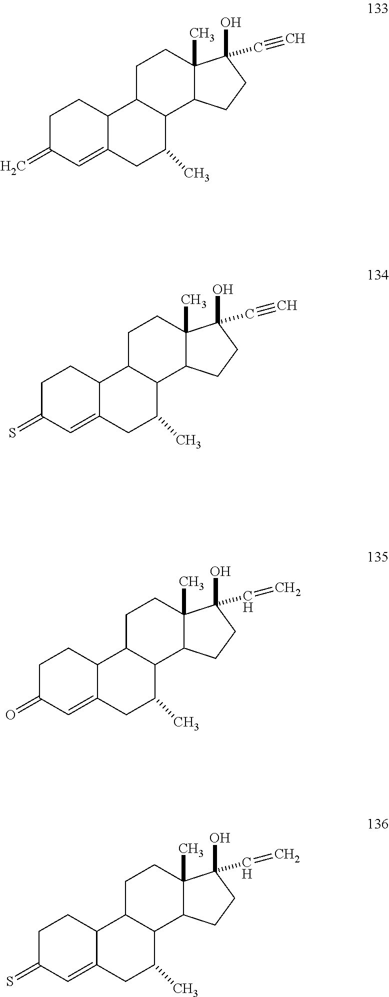 [SMILES: C#C[C@]1(O)CCC2C3C(CC[C@@]21C)C1CCC(=C)C=C1C[C@H]3C ; C#C[C@]1(O)CCC2C3C(CC[C@@]21C)C1CCC(=S)C=C1C[C@H]3C ; C=C[C@]1(O)CCC2C3C(CC[C@@]21C)C1CCC(=O)C=C1C[C@H]3C ; C=C[C@]1(O)CCC2C3C(CC[C@@]21C)C1CCC(=S)C=C1C[C@H]3C]

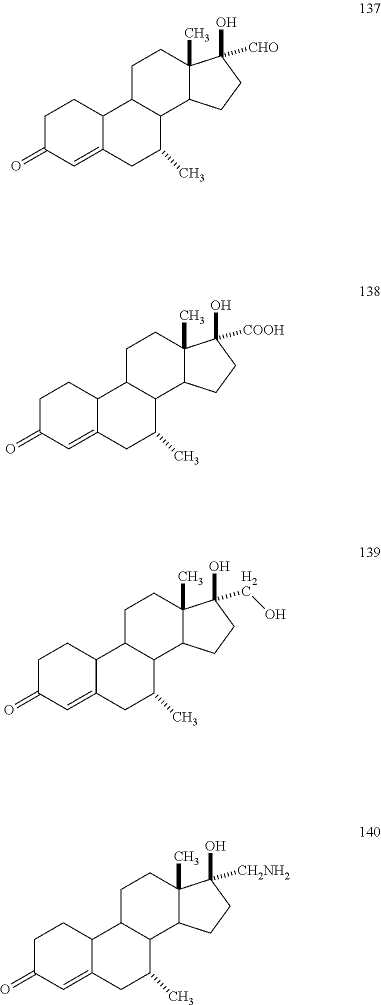

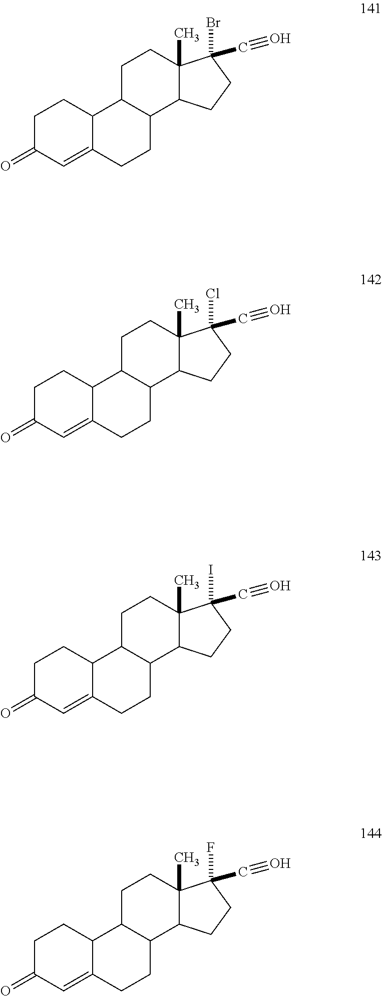

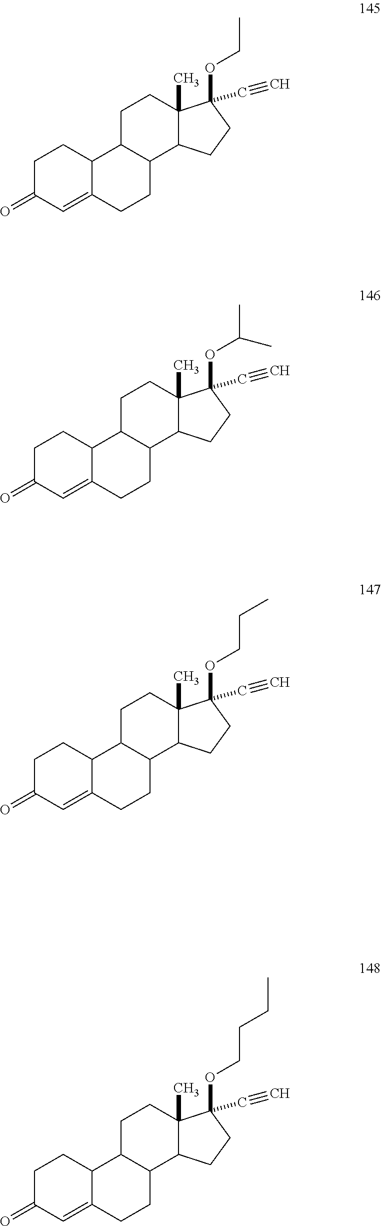 [SMILES: C#C[C@]1(OCC)CCC2C3CCC4=CC(=O)CCC4C3CC[C@@]21C ; C#C[C@]1(OC(C)C)CCC2C3CCC4=CC(=O)CCC4C3CC[C@@]21C ; C#C[C@]1(OCCC)CCC2C3CCC4=CC(=O)CCC4C3CC[C@@]21C ; C#C[C@]1(OCCCC)CCC2C3CCC4=CC(=O)CCC4C3CC[C@@]21C]

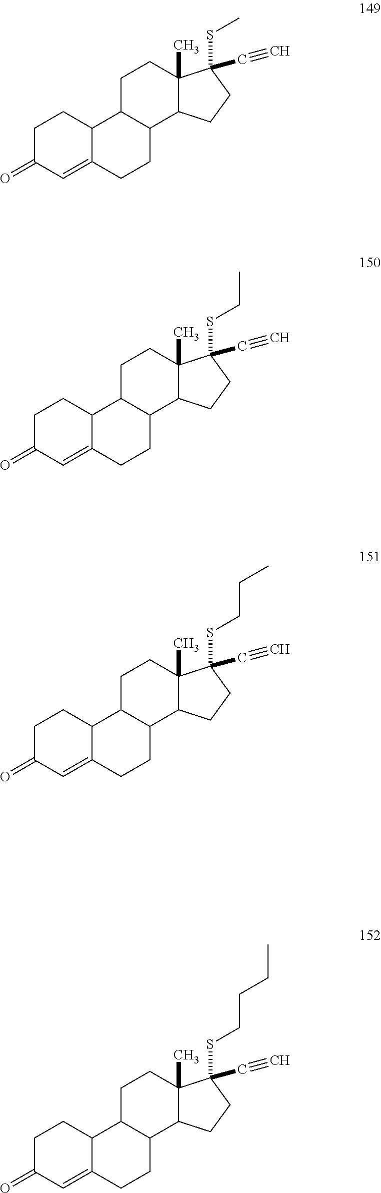 [SMILES: C#C[C@@]1(SC)CCC2C3CCC4=CC(=O)CCC4C3CC[C@@]21C ; C#C[C@@]1(SCC)CCC2C3CCC4=CC(=O)CCC4C3CC[C@@]21C ; C#C[C@@]1(SCCC)CCC2C3CCC4=CC(=O)CCC4C3CC[C@@]21C ; C#C[C@@]1(SCCCC)CCC2C3CCC4=CC(=O)CCC4C3CC[C@@]21C]

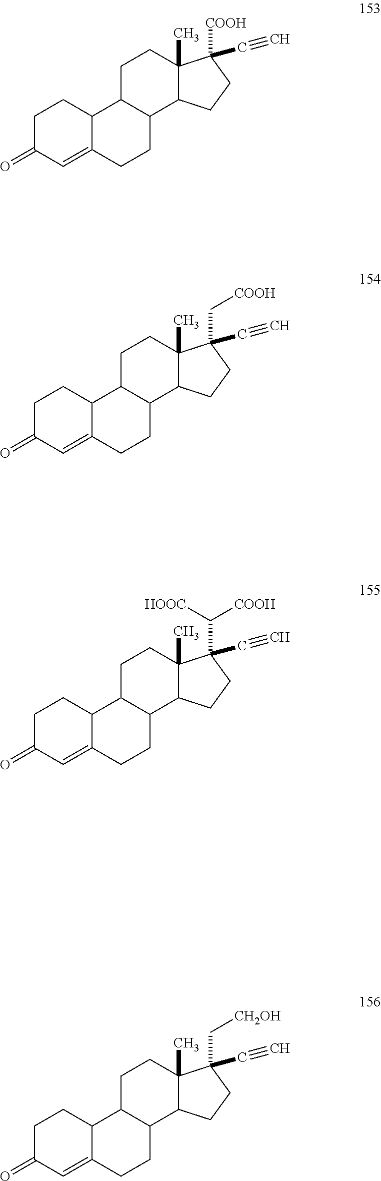 [SMILES: C#C[C@@]1(C(=O)O)CCC2C3CCC4=CC(=O)CCC4C3CC[C@@]21C ; C#C[C@@]1(CC(=O)O)CCC2C3CCC4=CC(=O)CCC4C3CC[C@@]21C ; C#C[C@@]1(C(C(=O)O)C(=O)O)CCC2C3CCC4=CC(=O)CCC4C3CC[C@@]21C ; C#C[C@@]1(CCO)CCC2C3CCC4=CC(=O)CCC4C3CC[C@@]21C]

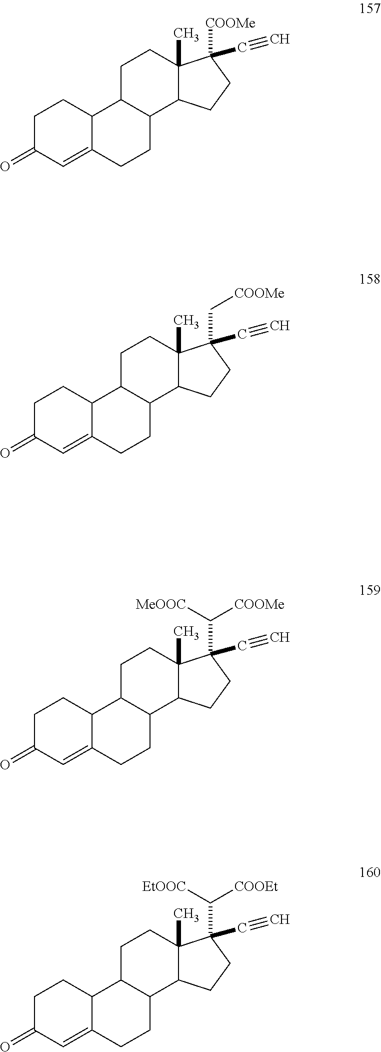 [SMILES: C#C[C@@]1(C(=O)OC)CCC2C3CCC4=CC(=O)CCC4C3CC[C@@]21C ; C#C[C@@]1(CC(=O)OC)CCC2C3CCC4=CC(=O)CCC4C3CC[C@@]21C ; C#C[C@@]1(C(C(=O)OC)C(=O)OC)CCC2C3CCC4=CC(=O)CCC4C3CC[C@@]21C ; C#C[C@@]1(C(C(=O)OCC)C(=O)OCC)CCC2C3CCC4=CC(=O)CCC4C3CC[C@@]21C]

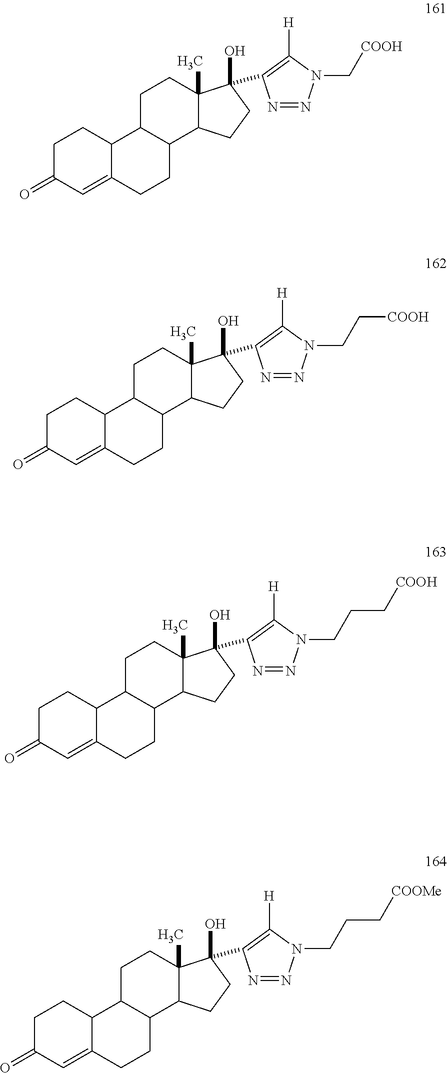 [SMILES: C[C@]12CCC3C4CCC(=O)C=C4CCC3C1CC[C@@]2(O)c1cn(CC(=O)O)nn1 ; C[C@]12CCC3C4CCC(=O)C=C4CCC3C1CC[C@@]2(O)c1cn(CCC(=O)O)nn1 ; C[C@]12CCC3C4CCC(=O)C=C4CCC3C1CC[C@@]2(O)c1cn(CCCC(=O)O)nn1 ; COC(=O)CCCn1cc([C@]2(O)CCC3C4CCC5=CC(=O)CCC5C4CC[C@@]32C)nn1]

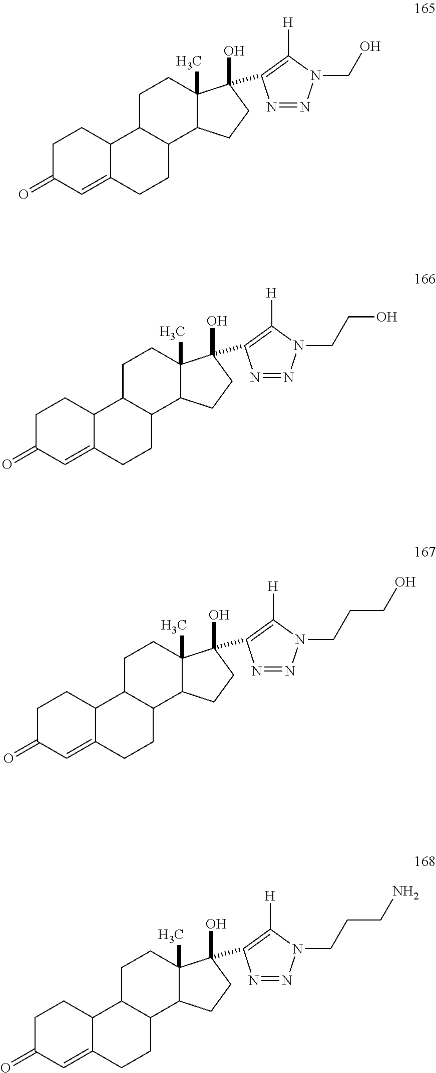 [SMILES: C[C@]12CCC3C4CCC(=O)C=C4CCC3C1CC[C@@]2(O)c1cn(CO)nn1 ; C[C@]12CCC3C4CCC(=O)C=C4CCC3C1CC[C@@]2(O)c1cn(CCO)nn1 ; C[C@]12CCC3C4CCC(=O)C=C4CCC3C1CC[C@@]2(O)c1cn(CCCO)nn1 ; C[C@]12CCC3C4CCC(=O)C=C4CCC3C1CC[C@@]2(O)c1cn(CCCN)nn1]

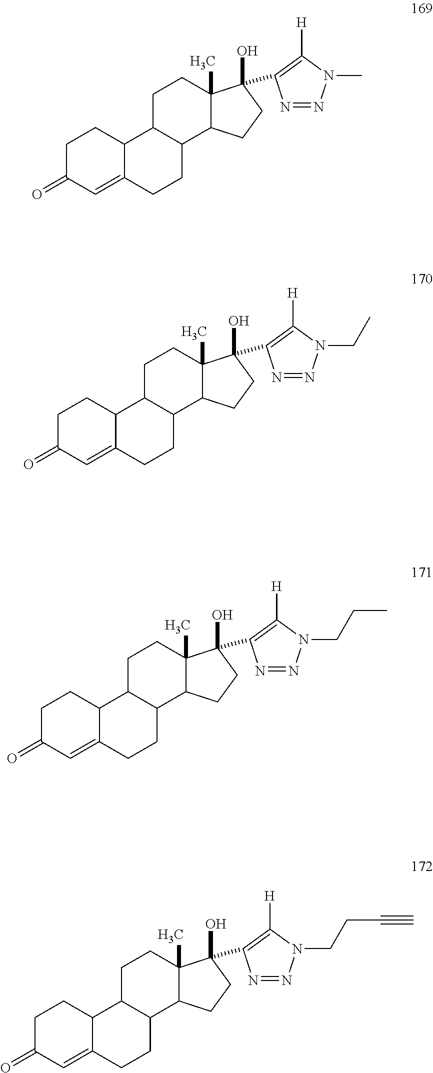 [SMILES: Cn1cc([C@]2(O)CCC3C4CCC5=CC(=O)CCC5C4CC[C@@]32C)nn1 ; CCn1cc([C@]2(O)CCC3C4CCC5=CC(=O)CCC5C4CC[C@@]32C)nn1 ; CCCn1cc([C@]2(O)CCC3C4CCC5=CC(=O)CCC5C4CC[C@@]32C)nn1 ; C#CCCn1cc([C@]2(O)CCC3C4CCC5=CC(=O)CCC5C4CC[C@@]32C)nn1]

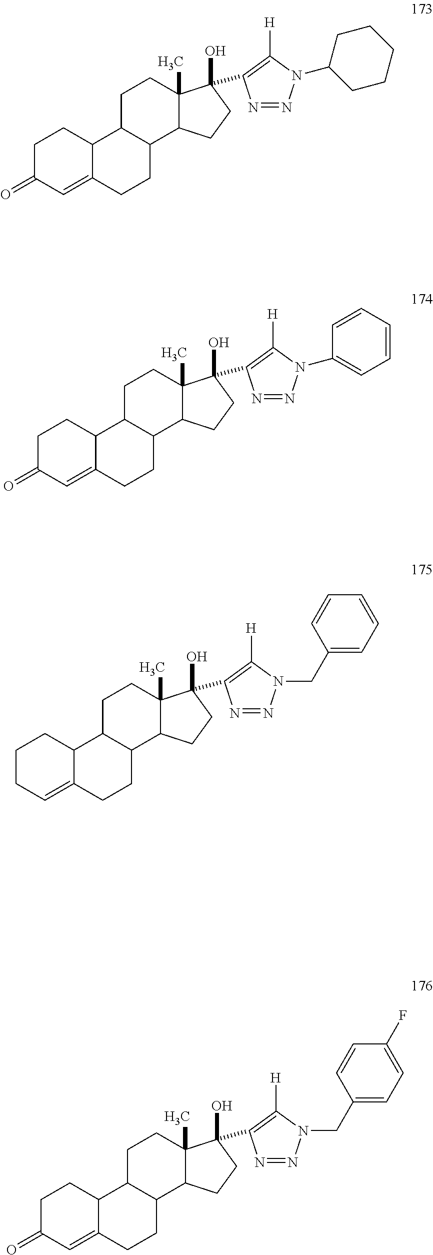 [SMILES: C[C@]12CCC3C4CCC(=O)C=C4CCC3C1CC[C@@]2(O)c1cn(C2CCCCC2)nn1 ; C[C@]12CCC3C4CCC(=O)C=C4CCC3C1CC[C@@]2(O)c1cn(-c2ccccc2)nn1 ; C[C@]12CCC3C4CCCC=C4CCC3C1CC[C@@]2(O)c1cn(Cc2ccccc2)nn1 ; C[C@]12CCC3C4CCC(=O)C=C4CCC3C1CC[C@@]2(O)c1cn(Cc2ccc(F)cc2)nn1]

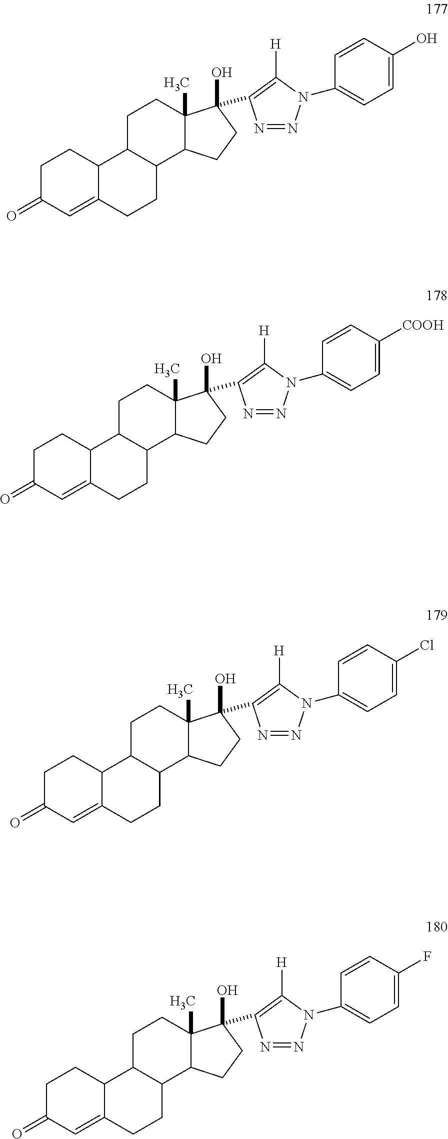 [SMILES: C[C@]12CCC3C4CCC(=O)C=C4CCC3C1CC[C@@]2(O)c1cn(-c2ccc(O)cc2)nn1 ; C[C@]12CCC3C4CCC(=O)C=C4CCC3C1CC[C@@]2(O)c1cn(-c2ccc(C(=O)O)cc2)nn1 ; C[C@]12CCC3C4CCC(=O)C=C4CCC3C1CC[C@@]2(O)c1cn(-c2ccc(Cl)cc2)nn1 ; C[C@]12CCC3C4CCC(=O)C=C4CCC3C1CC[C@@]2(O)c1cn(-c2ccc(F)cc2)nn1]

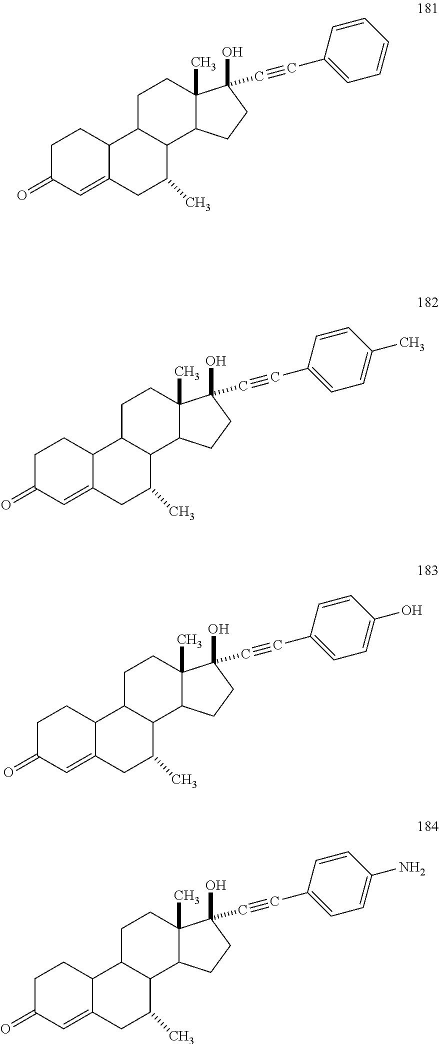 [SMILES: C[C@@H]1CC2=CC(=O)CCC2C2CC[C@@]3(C)C(CC[C@@]3(O)C#Cc3ccccc3)C21 ; Cc1ccc(C#C[C@]2(O)CCC3C4C(CC[C@@]32C)C2CCC(=O)C=C2C[C@H]4C)cc1 ; C[C@@H]1CC2=CC(=O)CCC2C2CC[C@@]3(C)C(CC[C@@]3(O)C#Cc3ccc(O)cc3)C21 ; C[C@@H]1CC2=CC(=O)CCC2C2CC[C@@]3(C)C(CC[C@@]3(O)C#Cc3ccc(N)cc3)C21]

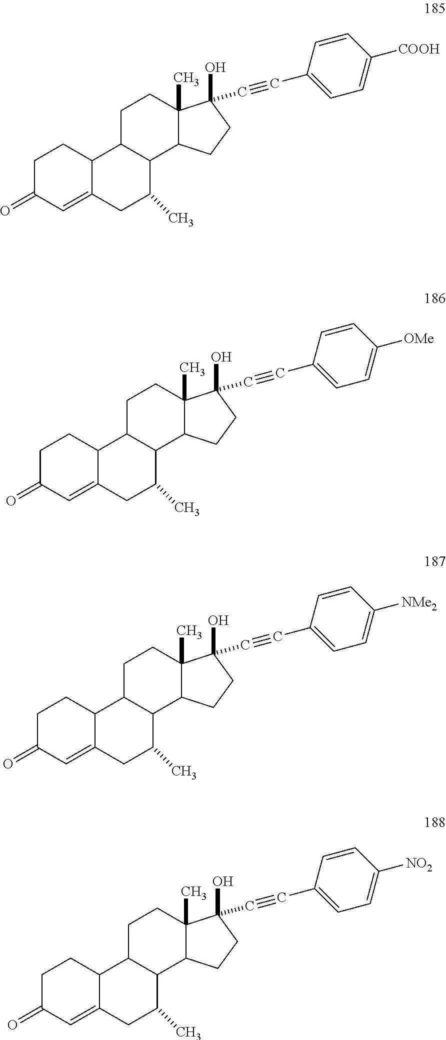 [SMILES: C[C@@H]1CC2=CC(=O)CCC2C2CC[C@@]3(C)C(CC[C@@]3(O)C#Cc3ccc(C(=O)O)cc3)C21 ; COc1ccc(C#C[C@]2(O)CCC3C4C(CC[C@@]32C)C2CCC(=O)C=C2C[C@H]4C)cc1 ; C[C@@H]1CC2=CC(=O)CCC2C2CC[C@@]3(C)C(CC[C@@]3(O)C#Cc3ccc(N(C)C)cc3)C21 ; C[C@@H]1CC2=CC(=O)CCC2C2CC[C@@]3(C)C(CC[C@@]3(O)C#Cc3ccc([N+](=O)[O-])cc3)C21]

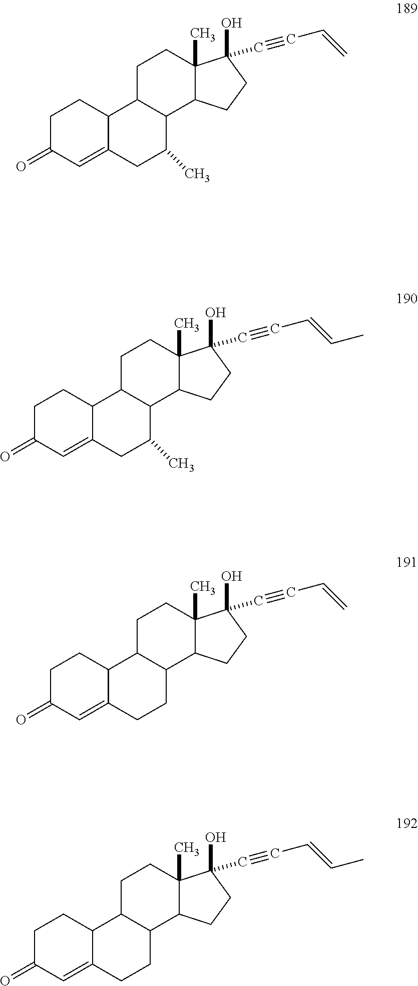 [SMILES: C=CC#C[C@]1(O)CCC2C3C(CC[C@@]21C)C1CCC(=O)C=C1C[C@H]3C ; C/C=C/C#C[C@]1(O)CCC2C3C(CC[C@@]21C)C1CCC(=O)C=C1C[C@H]3C ; C=CC#C[C@]1(O)CCC2C3CCC4=CC(=O)CCC4C3CC[C@@]21C ; C/C=C/C#C[C@]1(O)CCC2C3CCC4=CC(=O)CCC4C3CC[C@@]21C]

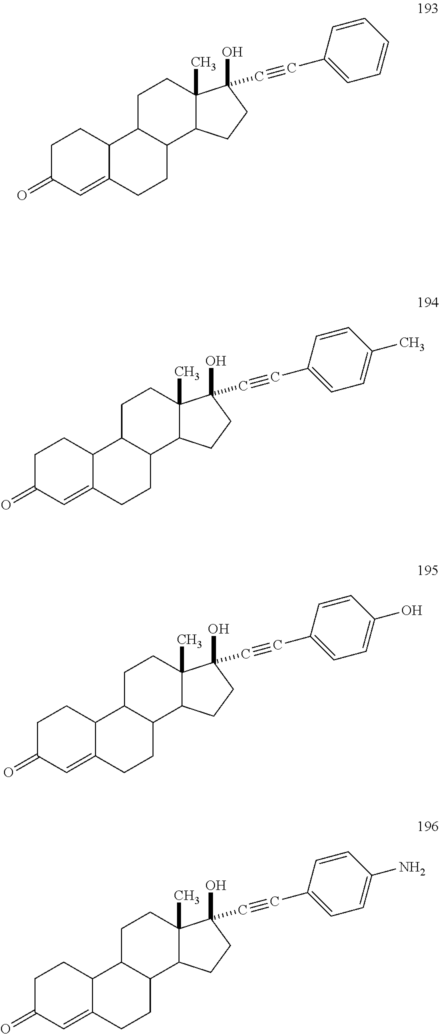 [SMILES: C[C@]12CCC3C4CCC(=O)C=C4CCC3C1CC[C@@]2(O)C#Cc1ccccc1 ; Cc1ccc(C#C[C@]2(O)CCC3C4CCC5=CC(=O)CCC5C4CC[C@@]32C)cc1 ; C[C@]12CCC3C4CCC(=O)C=C4CCC3C1CC[C@@]2(O)C#Cc1ccc(O)cc1 ; C[C@]12CCC3C4CCC(=O)C=C4CCC3C1CC[C@@]2(O)C#Cc1ccc(N)cc1]

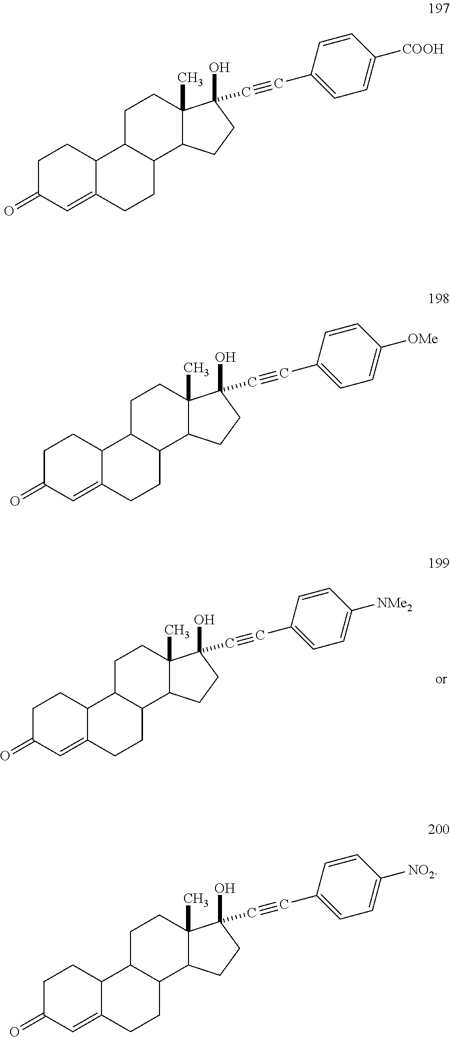 [SMILES: C[C@]12CCC3C4CCC(=O)C=C4CCC3C1CC[C@@]2(O)C#Cc1ccc(C(=O)O)cc1 ; COc1ccc(C#C[C@]2(O)CCC3C4CCC5=CC(=O)CCC5C4CC[C@@]32C)cc1 ; CN(C)c1ccc(C#C[C@]2(O)CCC3C4CCC5=CC(=O)CCC5C4CC[C@@]32C)cc1 ; C[C@]12CCC3C4CCC(=O)C=C4CCC3C1CC[C@@]2(O)C#Cc1ccc([N+](=O)[O-])cc1]

15. A pharmaceutical composition comprising one or more the compound of claim 1, or a pharmaceutically acceptable salt thereof, together with one or more diluents, excipients or carriers.

16. (canceled)

17. (canceled)

18. (canceled)

19. (canceled)

20. (canceled)

21. (canceled)

22. (canceled)

23. (canceled)

24. A method for treating a cancer patient, comprising the step of administering a therapeutically effective amount of one or more compounds, together with one or more carriers, diluents, or excipients, to a patient in need of relief from said cancer, at least one of the compounds having the formula: 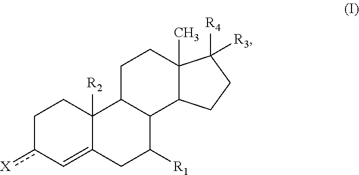
or a pharmaceutically acceptable salt thereof, wherein [symbol]
represents a single or double bond, wherein when [symbol] represents a single bond, X is a hydroxyl or alkyloxy; or when [symbol] represents a double bond, X is O, S, NH, N—OH, N—NH₂, or NR₇, wherein R₇ is an C1-C6 alkyl;
R₁ and R₂ are independently hydrogen, a C1 to C6 alkyl, alkenyl or alkynyl;
R₃ is hydrogen, hydroxyl, thiol, halo, azido, nitro, cyano, an alkyl, alkenyl, alkynyl, alkylalkynyl, alkyloxy, hydroxyalkyl, aminoalkyl, thiolalkyl, mercaptoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted; and
R₄ is hydrogen, hydroxyl, halo, azido, nitro, cyano, an alkyl, alkenyl, alkynyl, alkylalkynyl, alkyloxy, hydroxyalkyl, aminoalkyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

25. The method according to claim 24, wherein said cancer is castration resistant prostate cancer.
